SEQUENCE LISTING (49 sequences)

SEQ ID NO: 1 - DNA, 517 bp, Artificial Sequence
  Synthetic polynucleotide
tgacgcaattcaaccaagcgcgggtaaacggcgggagtaactatgactctctaaatagcaatatttacctttggagggaaaagttatcaggcatgcacctggtagctagtctttaaaccaatagattgcatcggtttaaaaggcaagaccgtcaaattgcgggaaaggggtcaacagccgttcagtaccaagtctcaggggaaactttgagatggccttgcaaagggtatggtaataagctgacggacatggtcctaaccacgcagccaagtcctaagtcaacagatcttctgttgatatggatgcagttcacagactaaatgtcggtcggggaagatgtattcttctcataagatatagtcggacctctccttaatgggagctagcggatgaagtgatgcaacactggagccgctgggaactaatttgtatgcgaaagtatattgattagttttggagtactcgtaaggtagccaaatgcctcgtcatctaattagtgacgcgcatgaatggatta

SEQ ID NO: 2 - RNA, 400 nt, Artificial Sequence
  Synthetic polynucleotide
nnnnnnngnnnnnaaaaguuaucaggcaugcaccugguagcuagucuuuaaaccaauagauugcaucgguuuaaaaggcaagaccgucaaauugcgggaaaggggucaacagccguucaguaccaagucucaggggaaacuuugagauggccuugcaaaggguaugguaauaagcugacggacaugguccuaaccacgcagccaaguccuaagucaacagaucuucuguugauauggaugcaguucacagacuaaaugucggucggggaagauguauucuucucauaagauauagucggaccucuccuuaaugggagcuagcggaugaagugaugcaacacuggagccgcugggaacuaauuuguaugcgaaaguauauugauuaguuuuggaguacucg

SEQ ID NO: 3 - DNA, 8 bp, Artificial Sequence
  Synthetic polynucleotide
tactagag

SEQ ID NO: 4 - RNA, 39 nt, Artificial Sequence
  Synthetic polynucleotide
acuugucacuacccugaccuaaannnnnnngnnnnnaaa

SEQ ID NO: 5 - RNA, 56 nt, Artificial Sequence
  Synthetic polynucleotide
acuugucacuacccugaccunnnnngaaannnnnnngnnnnnaaannnnnnnnnnn

SEQ ID NO: 6 - RNA, 12 nt, Artificial Sequence
  Synthetic polynucleotide
cuaacuaacuaa

SEQ ID NO: 7 - protein, 8 aa, Artificial Sequence
  Synthetic polypeptide
SNYGGGGS

SEQ ID NO: 8 - protein, 5 aa, Artificial Sequence
  Synthetic polypeptide
GGGGS

SEQ ID NO: 9 - RNA, 18 nt, Artificial Sequence
  Synthetic polynucleotide
gcuacuauuggcuaauaa

SEQ ID NO: 10 - RNA, 30 nt, Artificial Sequence
  Synthetic polynucleotide
gcuacuauuggcgcuacuauuggcuaauaa

SEQ ID NO: 11 - RNA, 30 nt, Artificial Sequence
  Synthetic polynucleotide
caaacaauaggagcuaguauuggcuaauaa

SEQ ID NO: 12 - RNA, 51 nt, Artificial Sequence
  Synthetic polynucleotide
gugagcaagggcgaggaggauaacauggccgacucucuaaauagcuaauaa

SEQ ID NO: 13 - RNA, 21 nt, Artificial Sequence
  Synthetic polynucleotide
gacucucuaaauagcuaauaa

SEQ ID NO: 14 - RNA, 36 nt, Artificial Sequence
  Synthetic polynucleotide
gcuaaaauuaaaggagacucucuaaauagcuaauaa

SEQ ID NO: 15 - RNA, 36 nt, Artificial Sequence
  Synthetic polynucleotide
gcaaaagcaaaagcagacucucuaaauagcuaauaa

SEQ ID NO: 16 - protein, 5 aa, Artificial Sequence
  Synthetic polypeptide
ATIGG

SEQ ID NO: 17 - protein, 9 aa, Artificial Sequence
  Synthetic polypeptide
ATIGATIGG

SEQ ID NO: 18 - protein, 9 aa, Artificial Sequence
  Synthetic polypeptide
QTIGATIGG

SEQ ID NO: 19 - protein, 16 aa, Artificial Sequence
  Synthetic polypeptide
VSKGEEDNNMADSLKV

SEQ ID NO: 20 - protein, 5 aa, Artificial Sequence
  Synthetic polypeptide
DSLKV

SEQ ID NO: 21 - protein, 10 aa, Artificial Sequence
  Synthetic polypeptide
AKIGGDSLKV

SEQ ID NO: 22 - protein, 10 aa, Artificial Sequence
  Synthetic polypeptide
AKAKADSLKV

SEQ ID NO: 23 - RNA, 13 nt, Artificial Sequence
  Synthetic polynucleotide
accgccaguagua

SEQ ID NO: 24 - RNA, 13 nt, Artificial Sequence
  Synthetic polynucleotide
accgccaguagua

SEQ ID NO: 25 - RNA, 13 nt, Artificial Sequence
  Synthetic polynucleotide
accgccaguagua

SEQ ID NO: 26 - RNA, 13 nt, Artificial Sequence
  Synthetic polynucleotide
uaccuuuggaggg

SEQ ID NO: 27 - RNA, 13 nt, Artificial Sequence
  Synthetic polynucleotide
uaccuuuggaggg

SEQ ID NO: 28 - RNA, 13 nt, Artificial Sequence
  Synthetic polynucleotide
uaccuuuggaggg

SEQ ID NO: 29 - RNA, 13 nt, Artificial Sequence
  Synthetic polynucleotide
uaccuuuggaggg

SEQ ID NO: 30 - RNA, 24 nt, Artificial Sequence
  Synthetic polynucleotide
uaugacucucuaaauagcaauauu

SEQ ID NO: 31 - RNA, 13 nt, Artificial Sequence
  Synthetic polynucleotide
uaccuuuggaggg

SEQ ID NO: 32 - RNA, 10 nt, Artificial Sequence
  Synthetic polynucleotide
guaagguagc

SEQ ID NO: 33 - RNA, 408 nt, Artificial Sequence
  Synthetic polynucleotide
nnnnnnngnnnnnaaaaguuaucaggcaugcaccugguagcuagucuuuaaaccaauagauugcaucgguuuaaaaggcaagaccgucaaauugcgggaaaggggucaacagccguucaguaccaagucucaggggaaacuuugagauggccuugcaaaggguaugguaauaagcugacggacaugguccuaaccacgcagccaaguccuaagucaacagaucuucuguugauauggaugcaguucacagacuaaaugucggucggggaagauguauucuucucauaagauauagucggaccucuccuuaaugggagcuagcggaugaagugaugcaacacuggagccgcugggaacuaauuuguaugcgaaaguauauugauuaguuuuggaguacucgnnnnnnnn

SEQ ID NO: 34 - RNA, 13 nt, Artificial Sequence
  Synthetic polynucleotide
acccugaccuaaa

SEQ ID NO: 35 - RNA, 13 nt, Artificial Sequence
  Synthetic polynucleotide
acacuuuggguca

SEQ ID NO: 36 - RNA, 12 nt, Artificial Sequence
  Synthetic polynucleotide
gaugguguucaa

SEQ ID NO: 37 - RNA, 20 nt, Artificial Sequence
  Synthetic polynucleotide
accugaccuaugguguucaa

SEQ ID NO: 38 - RNA, 17 nt, Artificial Sequence
  Synthetic polynucleotide
gaaaacacuuuggguca

SEQ ID NO: 39 - RNA, 13 nt, Artificial Sequence
  Synthetic polynucleotide
acacuuuggguca

SEQ ID NO: 40 - RNA, 13 nt, Artificial Sequence
  Synthetic polynucleotide
acacuuuggguca

SEQ ID NO: 41 - RNA, 15 nt, Artificial Sequence
  Synthetic polynucleotide
acccugaccuaaaaa

SEQ ID NO: 42 - RNA, 13 nt, Artificial Sequence
  Synthetic polynucleotide
acacuuuggguca

SEQ ID NO: 43 - RNA, 12 nt, Artificial Sequence
  Synthetic polynucleotide
gaugguguucaa

SEQ ID NO: 44 - RNA, 387 nt, Artificial Sequence
  Synthetic polynucleotide
nnaagcagnnnnnnnaannnnuaaaugcuagucggcnnnnnnnnnnuaagnnnnnnnnncggcggcgacaccgucaaauugcggggaauccnnnnuaaacccuuagcnaccnagcggccggggaaaggcggggguggccggaaugcgggggngguaaaanngcgagggaannnnnggacanuccgcagccaagccccaaagccnnnnnnnucnnnnngcuauggggaagguucacagacuaaauggggguggguugggnngcaancgcggcuuaagauauagucggunnnccgcggaaaggcgguccannnnnnnnnnnnnnaannnnnnnnnnnnugggnnnnnnnnnnnnnnnnnnnnnnnnnnnnnnnnnnnnnnnnauaaacg

SEQ ID NO: 45 - RNA, 387 nt, Artificial Sequence
  Synthetic polynucleotide
aanuaugncccgcgggnaugunggucuganacuucggguccgggaaaccaacuccgagcnccgauganguuuccuggucguucunanccuucugggunnguuacgcncuaucncucugguucangnccgccccacacugauugaaauuauagnuacccncccuauaauuucagggaagagacggcgcugggucuguuuccngnccccuuugugggggcaccccaaugcacccnngcagucgggcuucaagcuccgcuugcuuuggncguauccggagggcgaggcagcccgucgnuuguncgauguuncaggaggaaaguuancaccgagggacugnaaaaggcuacagcaaaacuaaaaggcggguguguugucgggganacccua

SEQ ID NO: 46 - RNA, 387 nt, Artificial Sequence
  Synthetic polynucleotide
aaaaguuaucaggcaugcaccugguagcuagucuuuaaaccaauagauugcaucgguuuaaaaggcaagaccgucaaauugcgggaaaggggucaacagccguucaguaccaagucucaggggaaacuuugagauggccuugcaaaggguaugguaauaagcugacggacaugguccuaaccacgcagccaaguccuaagucaacagaucuucuguugauauggaugcaguucacagacuaaaugucggucggggaagauguauucuucucauaagauauagucggaccucuccuuaaugggagcuagcggaugaagugaugcaacacuggagccgcugggaacuaauuuguaugcgaaaguauauugauuaguuuuggaguacucg

SEQ ID NO: 47 - RNA, 154 nt, Artificial Sequence
  Synthetic polynucleotide
ggagcaaugcuuugcgagauacccnnnnnnnnnccgaagguuauguacaggaannnnnnnnnnncucucuaaannnnnggguaucucgcaaagcauugcuccaaagguauuccuguacauaaccuucggnnnnnggaauaccuuuggagggnnn

SEQ ID NO: 48 - DNA, 6 bp, Artificial Sequence
  Synthetic polynucleotide
tactag

SEQ ID NO: 49 - RNA, 8 nt, Artificial Sequence
  Synthetic polynucleotide
cgaacuau

(19) United States
(12) Patent Application Publication
(10) Pub. No.: US 2010/0305197 A1
(43) Pub. Date: Dec. 2, 2010

(54) Conditionally Active Ribozymes And Uses Thereof

(75) Inventor: Austin J. Che, Cambridge, MA (US)

Correspondence Address: WOLF GREENFIELD & SACKS, P.C., 600 ATLANTIC AVENUE, BOSTON, MA, 02210-2206, US

(73) Assignee: Massachusetts Institute of Technology, Cambridge, MA (US)

(21) Appl. No.: 12/701,208

(22) Filed: Feb. 5, 2010

Related U.S. Application Data
(60) Provisional application No. 61/206,871, filed on Feb. 5, 2009.

(51) Int. Cl. A61K 31/7105; C07H 21/02; C12N 5/07; C12Q 1/68; G01N 33/50; A61P 35/00; A61P 37/02; A61P 31/00; A61P 5/00; A61P 33/00; A61K 48/00; G06G 7/58

(52) U.S. Cl. 514/44 R; 536/23.1; 435/325; 435/375; 435/6; 436/94; 703/11

(57) ABSTRACT

This invention relates, at least in part, to conditionally active ribozymes and uses of such ribozymes. Some aspects of this invention relate to the engineering of conditionally active ribozymes. In some embodiments, the splicing activity of such ribozymes is modulated by at least one regulatory element. Some aspects of this invention relate to uses of conditionally active ribozymes. RNA detection technology, conditional RNA expression technology, cell tagging technology, therapeutic approaches, and synthetic biology are examples of areas in which conditionally active ribozymes according to some aspects of the invention can be employed. RNA folding models useful in the design of conditionally active ribozymes with altered splicing efficiency and/or substrate specificity are provided. Compositions and methods to manufacture medicaments containing conditionally active ribozymes are also described.

### RELATED APPLICATIONS

This application claims the benefit 35 U.S.C. §119(e) of U.S. provisional application Ser. No. 61/206,871, filed Feb. 5, 2009, the entire disclosure of which is incorporated herein by reference.

### FEDERALLY SPONSORED RESEARCH

This invention was made with government support under Grant No. EEC-0540879, awarded by the NSF. The government has certain rights in this invention.

### FIELD OF THE INVENTION

Some aspects of this invention relate to the engineering and use of ribozymes, for example, conditionally active ribozymes.

### BACKGROUND OF THE INVENTION

### The Tetrahymena Ribozyme

The Tetrahymena ribozyme is a self-splicing intron found in the large subunit of the ribosomal RNA of Tetrahymena thermophila (Tetrahymena). It was the first discovered ribozyme and led to the Nobel Prize in Chemistry in 1989. FIG. 1 shows the cis-splicing reaction catalyzed by the ribozyme. The ribozyme excises itself while precisely ligating the two exons together. The precise ligation of the exons is determined by the internal guide sequence (IGS). The IGS base pairs with the two exons to direct accurate splicing (FIG. 2). The Tetrahymena ribozyme is a member of the family of group I introns [32]. Group I introns are split into five main subgroups called IA, IB, IC, ID, and IE, with additional subfamilies in these subgroups.

The Rfam database contains over 20,000 members in the group I family with the vast majority (over 95%) being classified in the IC3 subgroup [57, 175]. Most of the IC3 introns are found in the tRNALeu of the chloroplast of green plants (Viridiplantae). The IC1 subgroup, including the Tetrahymena ribozyme, is the next largest subgroup and many of its introns are found in rRNA. The naming of group I introns typically consists of three letters from the species name followed by the location of the intron [80]. The Tetrahymena ribozyme is called Tth.L1925, where Tth is from Tetrahymena thermophila, the L indicates the large subunit of the ribosomal RNA, and 1925 is the base location.

### Ribozyme Structure

Group I ribozymes typically have low primary sequence conservation, but they fold into a similar secondary structure [32, 163]. FIG. 3 shows the sequence and secondary structure of the Tetrahymena ribozyme. Although the ribozyme itself is 413 nt, the standard numbering labels the G_{α} added in the first step of splicing as 1, so the bases are numbered from 2 to 414. I only use nucleotides 28-414 from the native ribozyme. Helical double stranded regions are numbered sequentially with a P (paired region). Helical loops are labeled with L (loop region), and sequences between helices are denoted with J (joining region).

For example, L8 is the loop connecting the two strands forming P8 and J8/7 is the sequence connecting P8 and P7. To reduce ambiguity, domains labeled with a D. D₂, D₄₋₆, D_{3,7,8}, and D₉ herein, are the four major domains that form the ribozyme. For example, D₂ consists of P2 and P2.1 and D₄₋₆ consists of all of the P4, P5, P5abc, P6, and P6ab helices. The crystal structure for the active core of the Tetrahymena ribozyme has been solved [31, 54, 62, 143] and the complete crystal structure for the smallest known natural group I ribozyme from Azoarcus has also been published [1].

### Splicing

The biochemistry of the splicing reaction has been studied thoroughly [32]. The splicing reaction involves two transesterification reactions where phosphodiester bonds are transferred from one nucleotide to another (FIG. 4). The first step of splicing requires an exogenous guanosine or any of its 5′ phosphorylated forms (GMP, GDP, GTP). Both the 2′-OH and 3′-OH of the ribose on the guanosine are essential [34, 72]. The 3′-OH of the exogenous guanosine (G_{α}), attacks the 5′-splice site, disconnecting the 5′-exon from the ribozyme and leaving an end with a 3′-OH. The second step of splicing is the reverse of the first reaction and is another transesterification reaction. The 3′-OH of the 5′-exon attacks the last G of the ribozyme (G_{ω}), leading to the ligation of the 5′- and 3′-exons. The resulting ribozyme fragment contains G_{α}, on the 5′-end and G_{ω} on the 3′-end.

In the first step of splicing, the IGS pairs with the 5′-exon to form the P1 helix. This P1 helix contains an essential G:U base pair that determines the 5′-splice site. Some group I ribozymes missing the P1 segment do not splice [45], indicating the importance of this region in proper splicing. I use uₛ and G₆ to refer to the U splice site and the matching G in the IGS. The 6 indicates that the G is six bases upstream of the ribozyme. The G₆:uₛ wobble base pair is over 200-fold less stable than a G₆:C base pair [95], and this large destabilization likely contributes to catalysis. Base pairs other than G:U usually cannot substitute. For example, a G:C base pair shows a 25-fold reduction in k_{cat}, a 100-fold reduction in k_{cat}/Kₘ, and a reduced accuracy of splicing [122]. It is interesting that G:U wobble base pairs are not found in rRNA, perhaps because such a pair is more likely to be strained and the bond broken. In the second step of splicing, the IGS pairs with the 3′-exon to form the P10 helix.

The 3′-splice site is primarily determined by G_{ω} and internal ribozyme sequences, such as P9.0. For the native ribozyme, the P10 is neither necessary nor sufficient for recognition of the 3′ splice site, but it does increase the efficiency of splicing [89, 154]. In both steps of splicing, a guanosine (G_{α}, or G_{ω}) is bound by the ribozyme. The guanosine to binding site is located in P7 at the universally conserved G264:C311 base pair [96, 104]. This base pair binds guanosine as a triple base pair with a high affinity for binding (Kₘ=30 μM) [32].

### Trans-Splicing

Group I ribozymes can be engineered for trans-splicing, where the 5′-exon is on a separate RNA from the ribozyme and the 3′-exon (FIG. 5) [8, 92, 98, 126, 127, 139, 146]. FIG. 6 shows the details of the trans-splicing reaction. As in cis-splicing, the ribozyme finds the 5′-exon by the P1 base pairing. However, unlike cis-splicing, the P10 binding appears to be crucial. Disrupting the P10 base pairing eliminates trans-splicing activity in vivo [25]. Other experiments also show a significant drop (50 fold) in activity without a P10 match, but activity could be restored to 100% with only a 4 by P10 region complementary to the 3′-exon [89]. As the 5′-exon is not covalently attached to the ribozyme, the ribozyme needs to find and bind the 5′-exon. Inserting an antisense region complementary to the 5′-exon (about 45-100 nt) greatly increases effectiveness (50×) and specificity compared with no antisense region [7, 89].

### SUMMARY OF THE INVENTION

This invention relates, at least in part, to conditionally active ribozymes and uses of such ribozymes. Some aspects of this invention relate to the engineering of conditionally active ribozymes. In some embodiments, the splicing activity of such ribozymes is modulated by at least one regulatory element. Some aspects of this invention relate to uses of conditionally active ribozymes. RNA detection technology, conditional RNA expression technology, cell tagging technology, therapeutic approaches, and synthetic biology are examples of areas in which conditionally active ribozymes according to some aspects of the invention can be employed. RNA folding models useful in the design of conditionally active ribozymes with altered splicing efficiency and/or substrate specificity are provided. Compositions and methods to manufacture medicaments containing conditionally active ribozymes are also described.

According to some aspects of this invention, conditionally active ribozymes, comprising a catalytic RNA fragment that splices one or more RNA molecules, and at least one regulatory element modulating the activity of said catalytic RNA fragment, are provided. Some of the ribozymes provided according to some aspects of this invention catalyze a cis-splicing reaction. Some of the ribozymes provided according to some aspects of this to invention catalyze a trans-splicing reaction. Some of the ribozymes provided according to some aspects of this invention are derived from a group I intron or a group II intron. Some of the ribozymes provided according to some aspects of this invention are derived from a group I intron of Tetrahymena thermophila.

According to some aspects of this invention, conditionally active ribozymes the nucleotide sequence of which has been altered, are provided. According to some aspects of this invention, said nucleotide sequence alteration results in a change of the substrate specificity and/or the splicing activity of the ribozyme. The nucleotide sequence of the internal guide sequence (IGS) of some of the ribozymes provided according to some aspects of this invention is altered in at least one position. The nucleotide sequence of some of the ribozymes provided according to some aspects of this invention is altered based on the results of a computational RNA folding model calculating kinetic parameters of the splicing process. A computational RNA folding model as provided according to some aspects of this invention may employ, for example, a kinetic folding algorithm calculating the probability of splicing.

In some of the ribozymes provided according to some aspects of this invention the at least one regulatory element comprises a nucleic acid. In some of the ribozymes provided according to some aspects of this invention the at least one regulatory element comprises a nucleotide sequence that reversibly binds to said ribozyme. In some of the ribozymes provided according to some aspects of this invention the at least one regulatory element reversibly binds to the internal guide sequence (IGS) of said ribozymes, preferably to the reaction site. In some of the ribozymes provided according to some aspects of this invention said binding inhibits the splicing activity of the catalytic RNA fragment of said ribozyme.

According to some aspects, this invention provides conditionally active ribozymes comprising at least one regulatory element, said at least one regulatory element comprising at least one nucleotide sequence reversibly binding to a target molecule, said binding impairing the binding of said at least one regulatory element to said ribozyme.

A target molecule can be, for example, an amino acid, a peptide, a peptide or protein, a chemical compound, or a nucleic acid molecule. A target nucleic acid molecule can be, for example, a mRNA molecule, for example an endogenous mRNA molecule, or a RNA molecule transcribed from an artificial construct. Said artificial construct can be, for example, a constitutive construct or a conditional construct. A conditional construct can be, for example, a construct comprising a drug-responsive promoter, for example a doxicycline-inducible or repressible promoter, a tamoxifen-inducible or repressible promoter, or a promoter requiring DNA recombination to be activated or deactivated, such as mediated by to the cre-loxP system.

Some aspects of this invention relate to regulatory elements modulating the splicing activity of conditionally active ribozymes. These regulatory elements are also sometimes termed “gates”. Depending on regulatory element (or gate) design, conditionally active ribozymes can be engineered to be only active in the presence of one or more target molecules (for example ribozymes comprising YES, OR or AND gates). Further, ribozymes can be engineered to be only active in the absence of one or more target molecules (for example ribozymes comprising NOT or NOR gates).

Accordingly, some of the ribozymes provided according to some aspects of this invention comprise at least one regulatory element which comprises an anti-IGS region, flanked on both sides by regions antisense to said target nucleic acid molecule, wherein in the absence of said target nucleic acid molecule the anti-IGS region binds to the IGS and inhibits or prevents splicing and in the presence of said target nucleic acid said target molecule binds to said antisense regions resulting in the release of the anti-IGS:IGS binding and an enhancement or activation of splicing (YES gate).

Some of the ribozymes provided according to some aspects of this invention comprise at least one regulatory element which comprises an anti-IGS region, flanked on both sides by at least two regions antisense to at least two different target nucleic acid molecules, wherein in the absence of said target nucleic acid molecules the anti-IGS region binds to the IGS and inhibits or prevents splicing and in the presence of one or more of said target nucleic acid molecules said one or more target molecules bind to said antisense regions resulting in the release of the anti-IGS:IGS binding and an enhancement or activation of splicing (OR gate).

Some of the ribozymes provided according to some aspects of this invention comprise at least one regulatory element which comprises at least two anti-IGS regions, each flanked on both sides by o a region comprising an anti-anti-IGS region, said anti-anti-IGS region being flanked on both sides by regions antisense to said target nucleic acid molecule, wherein in the absence of said target nucleic acid molecule the anti-IGS region binds to the anti-anti-IGS region and enhances or activates splicing and in the presence of said target nucleic acid said target molecule binds to said antisense regions resulting in the release of the anti-IGS:anti-anti-IGS binding, resulting in the binding of the anti-IGS to the IGS and an inhibition or prevention of splicing (NOT gate).

Some of the ribozymes provided according to some aspects of this invention comprise at least one regulatory element which comprises at least two anti-IGS regions, each flanked on both sides by regions antisense to at least one target nucleic acid molecule per anti-IGS to region, wherein in the absence of said target nucleic acid molecule the anti-IGS region binds to the IGS and inhibits or prevents splicing and in the presence of said target nucleic acid said target molecule binds to said antisense regions resulting in the release of the anti-IGS:IGS binding and an enhancement or activation of splicing (AND gate).

According to some aspects of this invention, conditionally active ribozymes are provided in which the catalytic RNA fragment and the at least one regulatory element are part of the same RNA molecule. In some of the ribozymes provided according to some aspects of this invention the catalytic RNA fragment and the at least one regulatory element are separated by at least one spacer comprising a nucleotide sequence. Some of the conditionally active ribozymes provided according to some aspects of this invention comprise at least one additional element regulating the transcription and/or translation of nucleic acids. Transcriptional and/or translational termination signals are examples of such elements.

According to some aspects of this invention conditionally active ribozymes are provided that are not derivatives of a hammerhead ribozyme.

Sets of two or more conditionally active ribozymes are also provided according to some aspects of this invention. In some embodiments, a spliced nucleic acid generated as a result of the splicing activity of at least one conditionally active ribozyme in such a set is a target molecule of at least one other conditionally active ribozyme. According to some aspects of this invention, conditionally active ribozymes, or sets thereof, are generated from a library of modular and/or standardized fragments.

According to some aspects of this invention, nucleic acids coding for conditionally active ribozymes are provided. Such nucleic acids may comprise one or more additional elements that regulate the transcription and/or translation of nucleic acid sequences. Transcriptional and/or translational termination signals are examples of such elements.

This invention further relates, at least in part, to a cell or cells expressing at least one conditionally active ribozyme as described herein.

Aspects of this invention relate to kits comprising at least one conditionally active ribozyme as described herein and/or at least one nucleic acid coding for such a ribozyme, and/or at least one cell expressing at least one such ribozyme.

This invention further relates, at least in part, to methods using conditionally active ribozymes. According to some aspects of this invention, methods of splicing one or more RNA molecules are provided, comprising contacting one or more RNA molecules with at least one conditionally active ribozyme as described herein and/or a nucleic coding for at least one such ribozyme, wherein said conditionally active ribozyme splices said one or more to RNA molecules. According to some aspects of this invention, a conditionally active ribozyme may increase the native splicing of said one or more RNA molecules.

According to some aspects of this invention a conditionally active ribozyme exchanges at least one part of one or more RNA molecules with one or more RNA molecules of a different nucleotide sequence than said part of one or more RNA molecules. In some embodiments, the at least one part of the first one or more RNA molecules contains one or more mutations. In some embodiments, splicing mediated by a conditionally active ribozyme results in the generation of a transcript coding for a gene product. In some embodiments, one or more mutations cause a protein one or more RNA molecules code for to be impaired in its function and splicing mediated by a conditionally active ribozyme results in a restoration or an improvement of that function. In some embodiments, one or more mutations cause one or more RNA molecules to not be translated in full or in part and splicing mediated by a conditionally active ribozyme results in translation in full or in part of the one or more RNA molecules. This ribozyme mediated “repair-by-splicing” process is also sometimes termed “re-writing” of RNA.

According to some aspects of this invention, methods of changing the state of a cell are provided. Some of these methods comprise contacting a cell with a conditionally active ribozyme as described herein and/or a nucleic acid coding for a conditionally active ribozyme, whereby the conditionally active ribozyme changes the state of the cell. In some embodiments, the contacted cell expresses the target nucleic acid molecule of a conditionally active ribozyme. In some embodiments, the target molecule is an endogenous gene product specifically expressed in the contacted cell. In some embodiments, the expression a target molecule of a conditionally active ribozyme signifies a desirable or undesirable cell state.

In some embodiments, the change in the state of the cell comprises expression of a non-endogenous gene product, said expression being modulated by a conditionally active ribozyme's splicing activity. For example, said non-endogenous gene product may detectably label a cell or render a cell resistant to an antibiotic agent.

According to some aspects of this invention, methods using conditionally active ribozymes for the detection of target molecules in samples or cells are provided. Such methods may, according to some aspects of this invention, comprise contacting a sample with one or more conditionally active ribozyme as described herein and/or the nucleic acid coding for such a conditionally active ribozyme under conditions that allow said one or more conditionally active ribozyme to bind a target molecule, wherein said one or more conditionally active ribozyme comprises a regulatory element specifically binding a target to molecule, and said binding modulates the splicing activity of the catalytic RNA fragment of said at least one conditionally active ribozyme, said modulating leading to a detectable change in the state of said sample. In some embodiments, such a target molecule is a nucleic acid molecule. a protein, or a chemical compound.

According to some aspects of this invention, methods using conditionally active ribozymes for the detection of target molecules in a cell or a sample may further comprise detecting change mediated by a conditionally active ribozyme in a sample, wherein the presence or level of change in a cell or a sample is indicative of the presence or absence or the quantity of a target molecule in said cell or sample. In some embodiments, the change may be quantified, and, in some embodiments, the quantity of change determined in a cell or a sample is compared to a reference or control quantity of change. In some embodiments, the quantity of change in a cell or a sample is correlated to a relative or absolute amount of a target molecule in the cell or sample.

In some embodiments, detection methods comprise comparing the quantity of change in a cell or sample to the quantity of change in a reference or control cell or sample. In some embodiments, the presence or an elevated quantity of change in a cell or sample is indicative of the presence or an elevated amount of a target molecule in the cell or sample, the absence or a decreased quantity of change is indicative of the absence or a decreased amount of a target molecule in the cell or sample.

In some embodiments, the sample is a cell or tissue or body fluid sample from a subject. In some embodiments the presence and/or an increased quantity of change in a sample from a subject as compared to a reference or control sample indicates the presence of a condition in a subject, and the absence and/or a decreased quantity of change in said sample as compared to a reference or control sample indicates the absence of a condition in a subject. In some embodiments, the subject is a human subject.

In some embodiments, the target molecule of a conditionally active ribozyme is a viral transcript. In some embodiments, the presence and/or an increased quantity of change in a sample from a subject as compared to a reference or control sample is indicative of a viral infection in said subject.

In some embodiment, the contacting and/or detecting are performed in a cell-free reaction.

In some embodiments, the sample is an environmental sample and the presence of a target molecule in such a sample is indicative of the presence of an organism comprising or expressing said target molecule in said sample.

The invention further relates, at least in part, to the use of at least one conditionally active ribozyme in synthetic circuits or as part of linear RNA logic. In some embodiments, one or more conditionally active ribozyme functions as a RNA converter, and/or a signal adapter, and/or a RNA connector in such a synthetic circuit or as part of such linear RNA logic.

In some embodiments, the sample is a cell sample, and the target molecule is an endogenous gene product, for example a mRNA or a protein, of the cells contained in such a sample. In some embodiments, the presence and/or elevated amount of a target molecule in such a sample or absence and/or decreased amount of a target molecule in said sample indicates a specific state of said cells. In some embodiments, the cells in such a sample express a conditionally active ribozyme constitutively or inducibly. In some embodiments, such cells are useful for the manufacture of a product.

In some embodiments, the splicing activity of the catalytic RNA fragment of a conditionally active ribozyme leads to the generation of at least one new ribozyme. In some embodiments, the new ribozyme is of the same structure as the conditionally active ribozyme. In some embodiments, the new ribozyme is of a different structure as the conditionally active ribozyme. In some embodiments, any of these configurations result in a change of the quality of the detectable change in the sample.

In some embodiments, two or more conditionally active ribozymes are used in the methods described herein. In some such embodiments, the splicing activity of at least one of these two or more conditionally active ribozymes leads to the generation of a target molecule for at least one of the two or more conditionally active ribozymes. In some embodiments, the output of at least one such conditionally active ribozyme is the input of at least one other conditionally active ribozyme. In some embodiments, an amplification of the detectable change in a sample is achieved by using two or more conditionally active ribozymes in such a configuration. In some embodiments, any of these configurations result in a change of the quality of the detectable change in the sample.

According to some aspects of this invention, at least one of two or more conditionally active ribozymes in the configurations described above are chosen from a library of standardized conditionally active ribozymes.

The invention further relates, at least in part, to the use of conditionally active ribozymes in the therapy of diseases or conditions. According to some aspects of this invention, methods of such therapeutic use are provided. Some of these therapeutic methods comprise using a conditionally active ribozyme to treat a subject. In some embodiments, such to treatment comprises administering to a subject at least one conditionally active ribozyme as described herein and/or a nucleic acid coding for at least one such ribozyme and/or a composition comprising either at least one conditionally active ribozyme according to this invention and/or at least one nucleic acid coding for such a ribozyme. In some of these therapeutic methods, the splicing activity of a conditionally active ribozymes is modulated specifically by a target molecule indicative of a disease or condition and/or of an undesired cell state causally related to a disease or condition in said subject. In some embodiments, the modulation is an activation. In some embodiments activation of a conditionally active ribozyme results in a change of cells expressing said target molecule.

In some embodiments the change is expression of a cytotoxic or cytostatic protein or nucleic acid. In some embodiments, the change results in death or inhibition of proliferation of cells expressing a specific target molecule.

In some embodiments, the change is an exchange of at least one part of one or more RNA molecules with one or more RNA molecules of a different nucleotide sequence than said part of said one or more RNA molecules. In some embodiments, at least one part of said one or more RNA molecules contains one or more mutations. In some embodiments, said one or more mutations cause the protein the one or more RNA molecules code for to be impaired in its function and said change results in a restoration of said function. In some embodiments, said one or more mutations cause the one or more RNA molecules to not be translated in full or in part and said change results in translation in full or in part of the one or more RNA molecules.

In some embodiments, the change results in an amelioration of said disease or condition or of symptoms of said disease or condition.

In some embodiments, the disease or condition is an infectious disease, an autoimmune disease, a neoplastic disease, an endocrine autocrine or paracrine disease, a parasitic disease or a genetic disorder.

In some embodiments of this invention, the treated subject is a human subject.

According to some aspects of this invention, compositions comprising one or more conditionally active ribozymes as described herein and/or one or more nucleic acids coding for a conditionally active ribozyme as described herein and/or one or more cells expressing one or more conditionally active ribozymes as described herein. In some embodiments, such compositions comprise a pharmaceutically acceptable carrier.

According to some aspects of the invention, methods using one or more conditionally active ribozymes as described herein and/or one or more nucleic acids coding for a to conditionally active ribozyme as described herein and/or one or more cells expressing one or more conditionally active ribozymes as described herein in the manufacture of a medicament or a pharmaceutical composition for the treatment of a human disease or condition are provided.

This invention relates, at least in part, to methods of engineering conditionally active ribozymes with altered splicing efficiency and/or substrate specificity. According to some aspects of this invention, methods for generating such ribozymes based on computational RNA folding models are provided. Some of these methods comprise using computational RNA folding models to predict and/or model the splicing activity of one or more mutations and engineering at least one mutation in said ribozyme based on the results of said prediction and/or modeling results.

The subject matter of this application may involve, in some cases, interrelated products, alternative solutions to a particular problem, and/or a plurality of different uses of a single system or article.

Other advantages, features, and uses of the invention will become apparent from the following detailed description of non-limiting embodiments of the invention when considered in conjunction with the accompanying drawings, which are schematic and which are not intended to be drawn to scale. The claims are incorporated into this section by reference. In the figures, each identical or nearly identical component that is illustrated in various figures typically is represented by a single numeral. For purposes of clarity, not every component is labeled in every figure, nor is every component of each embodiment of the invention shown where illustration is not necessary to allow those of ordinary skill in the art to understand the invention. In cases where the present specification and a document incorporated by reference include conflicting disclosure, the present specification shall control.

### BRIEF DESCRIPTION OF THE DRAWINGS

FIG. 1. In the cis-splicing reaction, the ribozyme excises itself and some surrounding sequence from the RNA. The two exons are ligated by the ribozyme to form a spliced RNA. The internal guide sequence (IGS) helps determine the splice points.

FIG. 2. In its native context from Tetrahymena, the ribozyme splices out of ribosomal RNA. The internal guide sequence (IGS), shown in uppercase, base pairs first with the 5′-exon and then the 3′-exon, facilitating the precise ligation of the two exons. The color scheme is the same as FIG. 1.

FIG. 3. The secondary structure of the Tetrahymena ribozyme consists of paired helical regions P1-P10. The exon sequence is in lowercase. The IGS and exon sequences are shown using Ns to indicate the sequence flexibility available for engineering. The canonical numbering shown is based on the native ribozyme.

FIG. 4. Cis-splicing proceeds via two transesterification reactions. In the first step, the 3′-OH of an exogenous guanosine, G_{α} attacks the 5′-splice site, leaving a 3′-OH on uₛ. In the second step, the 3′-OH of the us attacks the 3′-exon, leading to ligation of the two exons. The P1 and P10 helices formed between the IGS and the exons help direct the two steps. The G₆:uₛ base pair is required for efficient splicing.

FIG. 5. The trans-splicing reaction performs the same reaction as in cis-splicing, except that the 5′-exon is on a separate RNA. The boxed antisense region is complementary to the region downstream of the target 5′-exon.

FIG. 6. Trans-splicing involves two transesterification reactions similar to the cis-splicing reactions in FIG. 4. An antisense region at the 5′-end of the ribozyme helps bring the target RNA near the ribozyme in the first step.

FIG. 7. In this cis-splicing GFP construct, the ribozyme self-splices itself out of the transcript, leaving an intact GFP. The amount of GFP fluorescence indicates the efficiency of splicing.

FIG. 8. The cis-splicing GFP construct has a rationally designed IGS0, ACACUUUGGGUCA. IGS0 forms 9 base pairs with the 5′-exon (P1 helix) and 4 base pairs with the 3′-exon (P10 helix). All mutations were made relative to the IGS0 sequence. In the absence of splicing, the boxed UAA stop codon guarantees that only half of GFP is translated. After splicing, the new UAU codon codes for a tyrosine that forms the fluorophore of GFP. The G₆:uₛ wobble base pair in the P1 helix determines the 5′-splice point.

FIG. 9. In this ordinary differential equation model of the cis-splicing GFP reaction, the names of the species are on the left and the reaction rates are labeled. All species except the GFP protein have a degradation term (δₙ) that includes both chemical degradation and irreversible non-productive side reactions.

FIG. 10. The splicing efficiency for all single mutations of IGS0 (ACACUUUGGGUCA) was calculated by normalizing to intact GFP. IGS0 is repeated once at every position. There is a large range of efficiencies even though all constructs are at most two mutations away from each other.

FIG. 11. Additional As inserted between the 5′-exon and the IGS serve as a spacer sequence and were not expected to add significant secondary structure.

FIG. 12. The cis-splicing GFP model in FIG. 9 was simplified to three steps and four parameters: f₁, p₂, f₃, and l₃.

FIG. 13. Using the model in FIG. 12, the four fit parameter values and the predicted splicing efficiencies are shown for the IGS variants. For comparison, the black horizontal bar represents the experimental splicing efficiency from FIG. 10. Also shown is the contribution from each step in the model (+: step 1, x: step 2, ◯: step 3). The overall efficiency is the product of these three efficiencies.

FIG. 14. The predicted and experimental splicing efficiencies for the IGS variants shows an R²=0:74.

FIG. 15. The contribution of the P1 folding step towards splicing efficiency is a sigmoidal function of the free energy of the P1 pairing. The efficiency saturates at energies above 12 kT.

FIG. 16. The structure information diagram from the group IC1 ribozyme alignment is mapped to the Tetrahymena secondary structure. At each position, the most common base (consensus) is shown.

FIG. 17. The second most common base is shown at each position in this structure information diagram. An upside down base indicates that it occurs less than the expected 25% frequency.

to FIG. 18. The sequence of a complete synthetic ribozyme is shown. Red bases indicate “harmless” positions and blue bases indicate “likely mutable” positions. Lowercase letters indicate bases that were swapped from the native ribozyme.

FIG. 19. For the synthetic ribozymes in Table 4, the number of nucleotides changed is plotted versus splicing efficiency. The splicing efficiency dropped relatively linearly as more nucleotides were changed.

FIG. 20. The structure information diagram from FIG. 16 is drawn using boxes instead of bases to highlight conserved regions of the ribozyme. Boxes are shown for all positions with an information content greater than 0.1 bits and a non-gap as the consensus.

FIG. 21. A trans-splicing ribozyme can knockdown a target RNA. In this example, the target RNA, X, is a coding sequence. The ribozyme inactivates X by inserting a premature stop codon into X while splitting the transcript in two. X is an antisense region complementary to the target RNA and is the anti-IGS.

FIG. 22. Trans-splicing implements a logical AND gate. The inputs in this case are X and the ribozyme construct. Both of the spliced outputs, X₁Y or X₂ depend on the logical AND of the inputs: [X₁Y] or [X₂]=[X]̂[ribozyme]

FIG. 23. The antisense length for a gfp-targeted trans-knockdown ribozyme was varied from 0-125 nt. A lower fluorescence indicates greater knockdown of gfp. The ribozyme with an 81 nt antisense region showed the greatest effect with about 40% knockdown.

FIG. 24. All knockdown ribozymes contained a 75 nt antisense region with a different anti-IGS sequence. The number indicates the expected number of base pairs in the anti-IGS:IGS pairing. The 9(3′) construct had an anti-IGS that base pairs with the 3′ end of the IGS, whereas the 9(5′) construct base pairs with the 5′ end of the IGS. 13(AS) formed 13 base pairs with the 3′-end of the antisense region, rather than the IGS.

FIG. 25. A constitutively expressed target lacZα was doubly transformed with a control (no ribozyme), with an active trans-knockdown ribozyme (anti(lacZ)), or with the anti(lacZ) ribozyme containing the G264A point mutation. The anti(lacZ) ribozyme knocked down to LacZ activity, even when splicing was inactivated by the G264A ribozyme mutant.

FIG. 26. A dual reporter plasmid expressing both gfp and lacZα was used as the target for all samples. The first column shows the target transformed with the reference plasmid. The second and third columns show the target transformed with a knockdown ribozyme targeting either gfp or lacZα. Both trans-knockdown ribozymes were specific for their intended target.

FIG. 27. A plasmid expressing both gfp and mcherry was used as the target for an anti(gfp) ribozyme. The ribozyme showed specificity in reducing GFP expression without affecting mCherry activity.

FIG. 28. The gfp→lacZα ribozyme targeted gfp, replacing the second half of gfp with lacZα. The pre-spliced lacZα did not contain start codons and could not be translated. The linker was designed so that the spliced lacZα was in-frame with the gfp fragment, producing a protein fusion with LacZ activity.

FIG. 29. The gfp→lacZα and mcherry→lacZα converter ribozymes were tested for trans-splicing. Activity is reported as normalized number of LacZ molecules For each of the converters, all conditions except one showed zero activity. There was activity only when the ribozyme splices on lacZα in reading frame 1, suggesting that splicing occurred at the expected site. A G264A inactive ribozyme mutant of the frame 1 converter showed no activity, indicating that the LacZ activity was due to splicing. No activity was detectable when only the target (gfp or mcherry) or only the converter ribozyme was present. Finally, there was no activity when the mismatched target was used with the frame 1 converter, indicating that the trans-splicing ribozymes were specific for their intended target.

FIG. 30. All constructs contained the target GFP. The first lacked any splicing ribozyme. The remaining constructs all contained identical ribozymes except for different 3′-exons. The anti(gfp) construct contained stop codons and the other three contained lacZα with linkers for the three different reading frames. The knockdown from the three gfp→lacZα ribozymes was independent of the linker's frame and was less efficient than the anti(gfp) knockdown ribozyme.

to FIG. 31. Anti-IGS variants of the mcherry→lacZα trans-splicing ribozyme were tested. The first four constructs varied the base in the anti-IGS that pairs with G₆ in the IGS. The first construct (G:C) is the same construct in FIG. 29 that showed activity. The fifth construct was missing the entire anti-IGS region. Tight binding of the anti-IGS to the IGS can inhibit trans-splicing.

FIG. 32. The boxed part is the standard splicing module. Any ribosome binding site (RBS) can be attached upstream and any coding sequence (CDS) can be attached down-stream. The two stop codons upstream of the IGS stop ribosomes from the upstream RBS from going through the ribozyme and into the coding sequence. Before splicing, due to the lack of a start codon and an RBS, the coding sequence will not be translated into a functional protein. After splicing, the RBS, start codon, and a standard leader peptide are joined (in frame) with the coding sequence. Thus, splicing allows the coding sequence to be translated. A standard splicing module can be optimized once and then reused with any RBS or coding sequence.

FIG. 33. Using GFP as a reporter, the standard splicing modules in Table 9 showed a range of splicing efficiencies (normalized to intact GFP).

FIG. 34. Version 6 of the standard module was tested with different coding sequences. After splicing, the coding sequence would have extra amino acids on the N-terminus. As the original intact coding sequence was used as a reference, the activity with the splicing module can be greater than one due to increased translational or folding efficiencies

FIG. 35. A standard splicing module was connected with the reporters GFP, mCherry, and LacZα. The data was background subtracted and normalized by the original intact reporter. All three reporters with an active splicing module showed activity higher than the intact reporter (efficiency>1). With an inactive ribozyme, GFP and LacZα showed no activity above background and mCherry showed near zero activity.

FIG. 36. A standard splicing module was tested with KanR, which confers resistance to kanamycin. Cells were streaked on LB agar plates containing either ampicillin or kanamycin. All constructs were on a plasmid with an ampicillin resistance gene. Cells containing an active splicing module were kanamycin resistant whereas those with an in-active splicing module were not kanamycin resistant. The positive control cells contained a plasmid with constitutively expressed KanR and the negative control cells contained a plasmid without KanR.

FIG. 37. The IGS in the version 6 standard module can pair with an incorrect site. Although this off-target pairing is weaker than the correct pairing, the G₆:uₛ wobble base pair is present which would allow erroneous splicing. This alternative splicing puts the coding sequence into the wrong reading frame for translation and expression.

FIG. 38. A biological transzystor is analogous to an electrical transistor. An electrical transistor (field-effect transistor shown) is a three terminal device. The gate, controlled by an input voltage, determines whether the source and drain terminals are connected. A biological transzystor, built from a splicing ribozyme, also has two states. The two exons are physically disconnected in the unspliced state, whereas they are connected when spliced. Splicing is controlled by a gate which is regulated by a third “input” RNA. Thus, the output RNA is spliced together only when the input RNA is present.

FIG. 39. Based upon standard splicing modules (FIG. 32), standard transzystors have a gate that detects an input RNA and regulates splicing. The flow of ribosomes in standard transzystors is analogous to the flow of electrons (current) in electrical transistors. The “source” region upstream of the transzystor generates ribosome flow. In the unspliced state, the ribosomes fall off at the stop codon. Thus, no ribosomes can reach the downstream “drain” region. In the spliced state, the stop codons have been removed, allowing ribosomes to flow from the source to the drain.

FIG. 40. The transzystor gate allows splicing only in the presence of a trans-RNA. The transzystor gate consists of an anti-IGS region surrounded on both sides by regions antisense to the input RNA. Without the input RNA, the anti-IGS base pairs with the IGS, forming a G₆:C base pair that prevents splicing. With the input RNA, the antisense regions pair with the input, releasing the anti-IGS:IGS pairing and allowing the IGS to pair with the 5′-splice site (5′-ss). The G₆:uₛ pairing leads to splicing. The order of the two antisense regions of the gate is flipped relative to the input to allow the G₆ and us to be near each other.

FIG. 41. A prototype transzystor gate was simulated using a kinetic folding program. The to input RNA and transzystor RNA were concatenated and initially folded independently and then together. The color scheme is the same as in FIG. 40. The G in the IGS, the C in the anti-IGS, and the U in the 5′-splice site are bolded. In the first third of the simulation, the transzystor and input RNAs were not allowed to interact. During this time, the transzystor gate sequestered the IGS, preventing splicing (top “off” state). When the input RNA was allowed to interact with the transzystor, the input RNA released the IGS:gate pairing, allowing the IGS to pair with the 5′-splice site (bottom “on” state).

FIG. 42. The gfp transzystor takes gfp as an input and produces lacZα as an output. Exogenously added AHL induces the input gfp expression through BBa_F2620. The input gfp RNA produces both GFP fluorescence and activates the gfp transzystor to produce LacZα. Thus, LacZ and GFP activities should be correlated.

FIG. 43. The mcherry input module should not activate the gfp transzystor. Thus, LacZ and mCherry activities should be uncorrelated.

FIG. 44. A gfp transzystor (gfp-1) was co-transformed either with an inducible gfp or mcherry input module. The graphs plot the normalized levels for input fluorescence versus output LacZ activity. The regression lines show that the transzystor responded linearly to the gfp input (left) but did not respond to the mcherry input (right).

FIG. 45. A mcherry transzystor was co-transformed either with an inducible gfp or mcherry input module. The graphs plot the normalized levels for input fluorescence versus output LacZ activity. The regression lines show that the transzystor responded linearly to the mcherry input (right) but did not respond to the gfp input (left).

FIG. 46. The normalized fluorescence from the gfp or mcherry input is plotted as a function of the AHL inducer concentration. The fluorescence of the inputs did not significantly change in the presence of a transzystor with a matching antisense region. Thus, both the gfp and mcherry transzystors have low input loading.

FIG. 47. The leakiness, low, and high states for the gfp and mcherry transzystors were measured for different inputs. LacZ activity is reported in units of equivalent LacZ molecules per A600 per μl. The leakiness measurements were made for each transzystor without any to input module. The remaining data shows the transzystors with an input module, either uninduced (low) or induced (high). The inactive ribozyme set used the matched input (e.g., gfp input for the gfp transzystor), but the ribozyme in the transzystor contained a point mutation that inactivates splicing. Both transzystors responded specifically to the induction of the matched input.

FIG. 48. Additional gfp transzystor variants were characterized using an inducible gfp input. The seven variants correspond to gfp-1 through gfp-7 in Table 10. Variant 1 is the same gfp transzystor characterized in FIG. 47 and Table 11. a): LacZ activity measured for leakiness, low and high states, b): dynamic range calculations, c): sensitivity calculations for various transzystor variants.

FIG. 49. The transzystor design is functionally modular. The standard splicing module, input gate, output, and even ribozyme can each be swapped in a modular manner.

FIG. 50. Transzystor gates can implement logic operations. All of these gate designs are variants of the basic gate in FIG. 40. In the NOT gate, splicing is inhibited by the input X. Instead of X controlling the pairing of the IGS with the 5′-splice site, X controls the pairing of the IGS with the IGS. In the OR gate, splicing is activated by either of the two inputs X or Y. The OR gate has interleaved antisense regions for X and Y. In the AND gate, splicing depends on both inputs X and Y. The AND gate consists of sequential gates for X and Y.

FIG. 51. Transzystors are universal RNA converters. The transzystor gate can detect any input RNA and output another RNA. I have demonstrated two working input gates using one output. Example 4 shows that other outputs can be seamlessly swapped into the standard splicing module.

### DETAILED DESCRIPTION OF THE INVENTION

Synthetic biologists aim to control biological systems in engineering new functions [48, 142]. Many engineered circuits have focused on regulating transcription [47, 53, 66, 137] and translation [77]. However, a surprise from sequencing the human genome is the small number of genes that follow a simple transcription to translation paradigm. Part of the explanation for how a complex organism can arise from few genes is RNA splicing [5]. At to least 75% of human genes are alternatively spliced [82, 111].

RNA splicing can be mediated by ribozymes. For example, some introns, such as class I introns, are self-splicing ribozymes that can excise themselves from an RNA strand they are on.

There are many natural examples of catalytically active RNA molecules or elements. In fact, the RNA world hypothesis posits that RNA was the first self-replicating molecule and some of the earliest organisms may have relied solely on RNA for replication and metabolism. Many remnants from this RNA world are still present today [132].

There are three general classes of biological components: input sensors, regulatory elements, and output actuators. Natural RNAs are multi-functional and can function in all three roles. As an input platform, RNAs can bind metabolites (riboswitches) [20, 21], sense temperature [156], and other RNAs [77, 94]. RNAs can both positively and negatively regulate transcription and translation, such as in the replication control of plasmid R1, control of RNA polymerases, or control of tRNA synthetase transcription by tRNAs. Catalytic RNAs can function as output effectors, with the most complicated catalytic RNA being the ribosome itself, found in all living cells. RNAs can further function as the input and the output of a biological process or a synthetic circuit.

For some applications in synthetic biology it is desirable to engineer all-RNA devices, where the inputs, outputs, and the active processing elements are all RNA. In an all-RNA circuit, device interconnections are simplified and components become interchangeable when a universal substrate is used. I engineered synthetic splicing systems and all-RNA devices for reading, processing, and writing RNA using various ribozymes. I show that such ribozymes are modular, easy to engineer, scalable, and multi-functional.

The term “ribozyme” (also termed “ribonucleic acid enzyme”, “RNA enzyme” or “catalytic RNA”), as used herein, refers to a nucleic acid-molecule or a complex of two or more nucleic acid molecules and, optionally, additional, non-nucleic acid components, with catalytic activity. In general, the nucleic acid type comprising a ribozyme is RNA and the nucleotides comprising said nucleic acid are ribonucleotides. The term “ribozyme” is also meant to refer to catalytically active nucleic acid molecules that contain a modified nucleotide or comprise a nucleic acid derivative. The term “ribozyme”, accordingly, can describe a RNA molecule that catalyzes a chemical reaction. It can also describe a RNA-derivative molecule that catalyzes a chemical reaction. Some natural ribozymes catalyze, for example, the hydrolysis of one of their own phosphodiester bonds, or the hydrolysis of bonds in other RNAs. Other ribozymes catalyze cis- or trans-splicing reactions. Ribozymes have also been found to catalyze the aminotransferase activity of the ribosome.

As known to one of skill in the art, it is possible to substitute one or more ribonucleotides of a RNA molecule with modified nucleotides without substantially affecting the structure or biological function of the molecule. The use of certain nucleic acid derivatives may, for example, increase the stability of the catalytically active nucleic acid molecules of this invention.

As used herein, a nucleic acid derivative is a non-naturally occurring nucleic acid or a unit thereof. Nucleic acid derivatives may contain non-naturally occurring elements such as non-naturally occurring nucleotides and non-naturally occurring backbone linkages.

Nucleic acid derivatives may contain backbone modifications such as but not limited to phosphorothioate linkages, phosphodiester modified nucleic acids, combinations of phosphodiester and phosphorothioate nucleic acid, methylphosphonate, alkylphosphonates, phosphate esters, alkylphosphonothioates, phosphoramidates, carbamates, carbonates, phosphate triesters, acetamidates, carboxymethyl esters, methylphosphorothioate, phosphorodithioate, p-ethoxy, and combinations thereof. The backbone composition of the nucleic acids may be homogeneous or heterogeneous.

Nucleic acid derivatives may contain substitutions or modifications in the sugars and/or bases. For example, they include nucleic acids having backbone sugars which are covalently attached to low molecular weight organic groups other than a hydroxyl group at the 3′ position and other than a phosphate group at the 5′ position (e.g., an 21-O-alkylated ribose group). Nucleic acid derivatives may include non-ribose sugars such as arabinose. Nucleic acid derivatives may contain substituted purines and pyrimidines such as C-5 propyne modified bases, 5-methylcytosine, 2-aminopurine, 2-amino-6-chloropurine, 2,6-diaminopurine, hypoxanthine, 2-thiouracil and pseudoisocytosine.

As used herein, the term “conditionally active ribozyme” refers to a ribozyme that is only active under a certain condition or certain conditions, for example in the absence or presence of an input, such as a target molecule.

As used herein, the term “target molecule” refers to a molecule, for example a nucleic acid, a protein, or a chemical compound, that can bind to a conditionally active ribozyme and said binding can regulate said ribozyme's catalytic activity. A target molecule can, accordingly, be referred to as the “input” of a conditionally active ribozyme or of a synthetic circuit or linear logic comprising such a ribozyme.

The nucleotide sequence of some ribozymes according to some aspects of this to invention are derived from the sequence found in naturally occurring ribozymes. Class I introns of Tetrahymena are examples for such naturally occurring ribozymes. The nucleotide sequence of the naturally occurring ribozyme has been altered in some ribozymes according to aspects of this invention to effect a change in the splicing activity and/or the substrate specificity of said ribozymes. A change in the nucleotide sequence in a part or parts of the ribozyme that mediate ribozyme:substrate interactions, for example the IGS, or in sequences that mediate the splicing reaction, are examples of such nucleotide sequence alterations. Addition or deletion of one or more nucleotides to or from any part or parts of a ribozyme, for example the addition of an anti-IGS region, or an anti-IGS region flanked by regions able to bind to a target nucleic acid or any type of regulatory element, are also examples of such alterations.

According to some aspects of this invention, nucleotide sequence alterations can be examined in silico. For example, a sequence alteration can be effected in a suitable software or algorithm and the resulting ribozyme can be modeled to determine the effect of said alteration on ribozyme structure and/or function. Thermodynamic algorithms, such as mfold or RNAfold (part of the Vienna RNA package, see Gruber et al., Nucleic Acids Res 2008), as well as kinetic algorithms, such as kinefold (Xayaphoummine et al., Nucleic Acids Res. 2005); are example of suitable algorithms for modeling ribozyme structure and/or function. In some embodiments of this invention that feature a splicing ribozyme, one aspect that can be examined using such algorithms is the probability of splicing. For example the probability of splicing exhibited by a ribozyme with an added nucleotide sequence representing a regulatory element, for example an anti-IGS region flanked by two regions capable of binding a target molecule, for example a target RNA molecule, can be calculated both in the presence and in the absence of said target molecule. One useful example of applying such algorithms is to calculate the probability of the correct G in the IGS pairing with the correct U at the splice site and compare said probability to the probability of the correct G in the IGS pairing with an incorrect U, i.e. not the correct U at the splice site. In some embodiments, values from modeling the presence and the absence of a target molecule can subsequently be compared. Based on the results from such modeling experiments, a ribozyme can be generated.

For example, in order to identify a desired regulatory element, a number of such elements can be designed and examined in silico. The element or elements displaying the most desirable characteristics can then be chosen and a correlating nucleotide sequence alteration can be effected in an actual ribozyme. As an example: in order to generate a to ribozyme comprising a regulatory element that activates the splicing activity of a conditionally active ribozyme only in the presence of a target molecule, for example a target RNA molecule, a regulatory element can be identified using the algorithms described herein, that shows a low probability of correct G:U pairing in the absence of said target molecule and a high probability of correct G:U pairing in the presence of said target molecule. After identifying and, if necessary, optimizing, such a regulatory element using the modeling approaches described herein, an actual ribozyme comprising the identified regulatory element can be generated by methods well known to those of skill in the art.

The substrate of splicing ribozymes is generally recognized and bound by nucleotide-nucleotide base pairing mediated binding. The nucleotide sequence or sequences involved in this ribozyme:substrate binding mediate the substrate specificity and, in some cases, at least part of the splicing efficiency of the ribozyme. In the case of group I intron derived ribozymes, this substrate: ribozyme interaction is, at least in part, mediated by the internal guide sequence (IGS) as described herein.

The bound substrate is converted in a reaction catalyzed by the ribozymes catalytically active fragment or fragments. In some exemplary embodiments, the reaction is a splicing reaction, resulting in the splicing of one or more RNA molecules. In some exemplary embodiments, the reaction is a hydrolysis reaction. The reaction product of a ribozyme according to some aspects of this invention, for example a spliced RNA molecule, is sometimes referred to as the “output” of a ribozyme.

In some embodiments, the input, the output and the ribozyme are all nucleic acids. In some embodiments they are all RNAs. In some embodiments, the output of a first ribozyme is also the input of a second ribozyme, said second ribozyme being of the same or a different structure and/or nucleotide sequence as the first ribozyme. In some embodiments, such a configuration of a set of two or more connected ribozymes is used to amplify a change in a cell or sample effected by a conditionally active ribozyme in response to an input. In some embodiments, one or more conditionally active ribozymes connect two logic circuits, wherein the output of one circuit is the input of at least one of said one or more ribozymes and the output of said one or more ribozymes is the input of the second logic circuit. Accordingly, conditionally active ribozymes according to aspects of this invention can function as signal adapters or connectors of logic circuits.

The term “logic circuit” refers to a switching circuit comprising at least one logic gate, at least one input and at least one output. A conditionally active ribozyme, activated by a target RNA molecule and generating a spliced reaction product, is an example of such a to logic circuit. The term “logic circuit” also refers to a logic element being part of a linear logic, for example involving a reaction having a start point or condition and an end point or condition. A logic circuit could, accordingly, be a conditionally active ribozyme converting an input into an output in either a reversible or irreversible manner.

In some embodiments, conditionally active ribozymes comprise modules. For example, a conditionally active ribozyme may comprise an input platform module, comprising, for example, a regulatory element binding a target molecule and regulating the ribozyme's catalytic activity, for example a YES gate, and an effector module, comprising, for example, a catalytically active region binding to a substrate and catalyzing a reaction involving said substrate. Some aspects of this invention relate to the generation of interchangeable modules mediating different functions of conditionally active ribozymes. Some aspects of this invention relate to the generation of standardized libraries of such modules that can easily be combined to generate new conditionally active ribozymes with new input and/or output characteristics. For example an input platform module specific for an input, for example a GFP mRNA, from a library of input platform modules can be combined with an output module specific for an output, for example mCherry mRNA, from a library of output modules. In some embodiments, such modules are generated, propagated and stored as DNA fragments coding for the respective ribozymal fragments. In some embodiments, DNA fragments are inserted and propagated in standard bacterial or other vectors using methods well known to the skilled artisan. In some embodiments, such modules are standardized, for example by using standardized restriction sites useful to combine modules, thus allowing for the efficient generation of conditionally active ribozymes with new input/output combinations from existing modules.

In some embodiments, a conditionally active ribozyme's activity leads to a change in the state of a cell or sample. In some embodiments, the change of the state of a cell or sample comprises activation or inhibition of expression of a gene product, endogenous or non-endogenous, said expression being modulated by a conditionally active ribozyme's splicing activity. In some embodiments, a non-endogenous gene product may detectably label a cell or render a cell resistant to an antibiotic agent. Detectably labeling a cell may comprise, for example, expressing a fluorescent protein, such as GFP or mCherry, in said cell. Expressing an endogenous or non-endogenous marker gene that can readily be detected by antibodies or by measuring its activity is another example of such a labeling strategy. In some embodiments, such marker genes code for surface markers of cells. Cells expressing such surface markers can be labeled, quantified, separated or enriched using various to immunological methods well known to those of skill in the related arts.

Antibiotic agents are well known to those of skill in the art. Kanamycin, ampicillin, neomycin, hygromycin, zeocin, blasticidin, and puromycin are examples of antibiotic agents suitable to kill responsive prokaryotic and/or eukaryotic cells. Gene products rendering cells resistant to specific antibiotic agents, such as the bla gene product for ampicillin resistance, the pac gene product for puromycin resistance, and the ble gene product for zeocin resistance, are well characterized and well-known to those of skill in the art.

A “sample”, as used herein, may be a biological sample, an environmental sample or an artificial sample. A biological sample may be a sample from a subject such as a bodily fluid or tissue sample. The term tissue as used herein refers to both localized and disseminated cell populations including but not limited to brain, heart, breast, colon, bladder, uterus, prostate, stomach, testis, ovary, pancreas, pituitary gland, adrenal gland, thyroid gland, salivary gland, mammary gland, kidney, liver, intestine, spleen, thymus, bone marrow, trachea and lung. Biological fluids include saliva, sperm, serum, plasma, blood, lymph and urine, but are not so limited.

An environmental sample may be but is not limited to an air sample, a water, or a soil sample. An artificial sample may be generated by manufacture of artificial, biological, or other components.

As used herein, a “subject” is preferably a human, non-human primate, or other mammal, for example a cow, horse, pig, sheep, goat, dog, cat or rodent. In all embodiments, human subjects are preferred.

Some aspects of the invention relate to the detection of a target molecule or input by determining the presence or amount or level of said molecule in a sample.

According to some aspects of this invention, this determination is performed by assaying a sample for the presence or the quantity of said target molecule or input as described herein using conditionally active ribozymes, nucleic acids encoding such ribozymes or cells expressing such ribozymes, as provided by this invention.

The presence or level of a target molecule or input may be determined by contacting a sample with a conditionally active ribozyme according to this invention under conditions allowing said ribozyme to bind said target molecule and to catalyze a reaction. Subsequently, the sample can be assayed for the result, or output, of said reaction, for example an immediate result of a splicing reaction may be the generation of a specific RNA molecule. In some embodiments, for example if a conditionally active ribozyme is expressed in a cell, one result of a splicing reaction can be the expression of a protein, such as a marker protein, for to example a fluorescent protein.

Examples of preferred methods for the detection of ribozyme reaction products, or output molecules, include, but are not limited to, nucleotide amplification or hybridization based methods from the list of polymerase chain reaction (PCR), reverse transcriptase (RT)-PCR, northern blotting, Southern blotting, quantitative sequencing methods, such as SOLEXA or 454 sequencing, and microarray analysis.

Examples of preferred methods for the detection of ribozyme reaction products, or output molecules, include, but are not limited to, immunologically based assay methods from the list of immunohistochemistry, western blotting assay, enzyme-linked immunosorbent assay (ELISA), enzyme-linked immunospot assay (ELISPOT), lateral flow test assay, enzyme immunoassay (EIA), fluorescent polarization immunoassay (FPIA), chemiluminescent immunoassay (CLIA), antibody sandwich capture assay, or isoelectric focusing (IEF) assay, fluorescence activated cell sorting (FACS), and magnetic cell sorting (MACS).

Some methods of determining the presence and/or level of a target molecule in a cell or sample may include use of labels to monitor the presence of cells expressing or comprising said target molecule. Examples of labels include, but are not limited to fluorescent labels, radiolabels or chemiluminescent labels, which may be utilized to determine whether a target molecule is present in cell or sample, and/or to determine the level of said target molecule in said cell or sample. These and other in vitro and in vivo imaging methods for determining the presence and/or level of a target molecule in a cell or sample are well known to those of ordinary skill in the art.

In some embodiments, the results of such target molecule detection procedures can be used to diagnose a disease or condition in a subject. For example, the presence of a target molecule signifying such a disease or condition in a sample obtained from a subject would indicate said subject as having said disease or condition. Likewise, an aberrant level of a target molecule in a sample from a subject would indicate said subject as having a disease or condition, if said aberrant level signifies said disease or condition. The presence of a target molecule in a sample from a subject that is not expected to contain said target molecule would be an example of an “aberrant level” of a target molecule. For example the presence of a viral nucleic acid in a body fluid or tissue sample of a subject is indicative of a viral infection of said subject. Likewise, a significantly higher or lower level of a target molecule than expected is another example of an “aberrant level” of a target molecule.

In some embodiments, the level of a target molecule as determined using any of the to methods employing conditionally active ribozymes as described herein, is compared to a control or reference level. Generally, the control or reference level will reflect an average level expected to be exhibited by a suitable control sample. For example, in the case of a sample from a subject to be tested for the presence or level of a target molecule signifying a disease or condition, the control or reference level would preferably reflect the average level of said target molecule expected in individuals not indicated to have said disease or condition. As an example, a control sample from an individual known to be healthy could be assayed in parallel to the actual experimental or diagnostic sample. Or, alternatively or additionally, an artificial sample containing a known level of the target molecule reflecting a level representative of a level expected in an individual not indicated to have said disease or condition could be used as a control sample. Historical data or data from a number of control samples that have been averaged can also be useful to compare to the actual data from an actual sample.

The control, or reference, or baseline level can be determined using standard methods known to those of skill in the art. Examples of standard methods include, for example, assaying a number of samples from subjects that are clinically normal in respect to the disease or condition in question and determining the average level of a specific target molecule for the samples.

The design of the detection procedure, the choice of a suitable sample, and the choice of suitable controls, will depend on the target molecule to be detected.

In some embodiments, the invention provides kits comprising ribozymes or nucleic acids coding for ribozymes according to aspects of this invention.

An example of such a kit may include one or more conditionally active ribozymes, or nucleic acids coding for such ribozymes. As an option, a kit according to some embodiments of the invention may include one or more control samples. As used herein the term “control sample” typically means a sample tested in parallel with the experimental materials, although a control sample may be tested separately from experimental materials, and may reflect a historical control value. Examples of control samples include, but are not limited to, actual samples from a control specimen or samples generated through manufacture to be tested in parallel with the experimental samples. In some embodiments, a kit may include a positive control sample and/or a negative control sample. For example, in case of a diagnostic kit, the negative control will be based on apparently healthy individuals in an appropriate age bracket. A positive control, for example based on individuals indicated as having the disease or condition signified by the target molecule to be assayed or generated through manufacture, can be used to verify experimental procedures. Alternatively, a positive control can comprise a sample containing isolated target molecule.

The foregoing kits can include instructions or other printed material on how to use the various components of the kits.

Any of the terms “therapy”, “therapeutic use”, “therapeutic method”, “treatment” or “treating”, are intended to include one or more clinical interventions with an intent to induce prophylaxis, amelioration, prevention or cure of a condition (e.g., a viral infection). Treatment or therapy after a condition (e.g., a viral infection) has been diagnosed or clinically manifested aims to reduce, ameliorate or altogether eliminate the condition, and/or its associated symptoms, or prevent it from becoming worse. Treatment or therapy of subjects before a condition (e.g., a viral infection) has been diagnosed or clinically manifested (e.g., prophylactic treatment) aims to reduce the risk of developing the condition and/or lessen its severity if the condition does develop. As used herein, the term “prevent” refers to the prophylactic treatment of a subject who is at risk of developing a condition (e.g., a viral infection) resulting in a decrease in the probability that the subject will develop the disorder, and/or to the inhibition of further development of an already established disorder.

As used herein, a treatment may be prophylactic and/or therapeutic. In some embodiments, a treatment may include preventing disease development or progression. In certain embodiments, a treatment may include inhibiting and or reducing the rate of disease development or progression. It should be appreciated that the terms preventing and/or inhibiting may be used to refer to a partial prevention and/or inhibition (e.g., a percentage reduction, for example about 5%, about 10%, about 20%, about 30%, about 40%, about 50%, about 60%, about 70%, about 80%, about 90%, or higher or lower or intermediate percentages of reduction). However, in some embodiments, a prevention or inhibition may be complete (e.g., a 100% reduction or about a 100% reduction based on an assay or an expected progression).

The term “cytotoxic or cytostatic protein or nucleic acid” refers to proteins or nucleic acids that, when contacted with a cell, will either kill or inhibit the proliferation of said cell. This effect can either be achieved directly, for example by triggering a cell death pathway in the cell, or indirectly, for example by changing said cell in a way that makes it a target for other cells that kill said cell. Such proteins are known to those of skill in the art and suitable cellular pathways, for example apoptotic pathways, are readily identifiable by those of skill in the art. “Cytotoxic or cytostatic nucleic acids” can be nucleic acids coding for cytotoxic or cytostatic proteins, such a mRNAs. They can also be nucleic acids leading to the knockdown to of gene products essential for survival or proliferation of said cell. Antisense RNAs and shRNAs are examples of such knockdown-capable nucleic acids. Gene products essential for survival or proliferation, for example many housekeeping genes, are readily identifiable for those of skill in the art.

According to some aspects of the invention, compositions containing a ribozyme or a nucleic acid coding for a ribozyme or a cell expressing a ribozyme according to aspects of this invention are provided. The compositions may contain any of the foregoing (as a therapeutic agent) in an optional pharmaceutically acceptable carrier. Thus, in related aspects, some embodiments of the invention provide a method for forming a medicament that involves placing a therapeutically effective amount of the therapeutic agent in the pharmaceutically acceptable carrier to form one or more doses.

The effectiveness of treatment or prevention methods of the invention can be determined using standard diagnostic methods well known to the of skill in the related medical arts.

Therapeutic compositions of the present invention are administered in pharmaceutically acceptable preparations. Such preparations may contain pharmaceutically acceptable concentrations of salt, buffering agents, preservatives, compatible carriers, supplementary immune potentiating agents such as adjuvants and cytokines, and optionally other therapeutic agents.

As used herein, the term “pharmaceutically acceptable” means a non-toxic material that does not interfere with the effectiveness of the biological activity of the active ingredients. The term “physiologically acceptable” refers to a non-toxic material that is compatible with a biological system such as a cell, cell culture, tissue, or organism.

The characteristics of the carrier will depend on the route of administration. Examples of physiologically and pharmaceutically acceptable carriers include, without being limited to, diluents, fillers, salts, buffers, stabilizers, solubilizers, and other materials which are well known in the art. The term “carrier” denotes an organic or inorganic ingredient, natural or synthetic, with which the active ingredient is combined to facilitate the application. The components of the pharmaceutical compositions also are capable of being co-mingled with the molecules of the present invention, and with each other, in a manner such that there is no interaction which would substantially impair the desired pharmaceutical efficacy.

Therapeutics according to some embodiments of the invention can be administered by any conventional route, for example injection or gradual infusion over time. The administration may, for example, be oral, intravenous, intratumoral, intraperitoneal, intramuscular, intracavity, subcutaneous, or transdermal. An exemplary route of administration is by pulmonary aerosol. Techniques for preparing aerosol delivery systems are well known to those of skill in the art. Generally, such systems should utilize components which will not significantly impair the biological properties of the therapeutic agent (see, for example, Sciarra and Cutie, “Aerosols,” in Remington's Pharmaceutical Sciences, 18th edition, 1990, pp 1694-1712). Those of skill in the art can readily determine the various parameters and conditions for producing aerosols without undue experimentation.

The compositions of some embodiments of the invention are administered in effective amounts. An “effective amount” is that amount of a composition that alone, or together with further doses, produces the desired response. In some cases, the desired response is prevention of a disease. In some cases of treating a particular disease or condition the desired response is inhibiting the progression of the disease. This may involve slowing the progression of the disease temporarily, although more preferably, it involves halting the progression of the disease permanently. In some cases, the desired response to treatment can be delaying or preventing the manifestation of clinical symptoms characteristic for the disease or condition.

The effect of treatment can be monitored by routine methods or can be monitored according to diagnostic methods of the invention discussed herein. The effective amount will depend, of course, on the particular condition being treated, the severity of the condition, the individual patient parameters including age, physical condition, size and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reason.

Pharmaceutical compositions according to some embodiments of this invention some of which are exemplified in the foregoing methods preferably are sterile and contain an effective amount of one or more therapeutic agents as described herein for producing the desired response in a unit of weight or volume suitable for administration to a patient.

The doses of one or more therapeutic agents as described herein administered to a subject can be chosen in accordance with different parameters, in particular in accordance to with the mode of administration used and the state of the subject. Other factors include the desired period of treatment. In the event that a response in a subject is insufficient at the initial doses applied, higher doses (or effectively higher doses by a different, more localized delivery route) may be employed to the extent that patient tolerance permits.

Administration of therapeutic compositions to mammals other than humans, e.g. for testing purposes or veterinary therapeutic purposes, is carried out under substantially the same conditions as described above.

The pharmaceutical compositions may contain suitable buffering agents, for example acetic acid in a salt, citric acid in a salt, boric acid in a salt, and/or phosphoric acid in a salt.

The pharmaceutical compositions also may contain, optionally, suitable preservatives, such as: benzalkonium chloride, chlorobutanol, parabens and/or thimerosal.

The pharmaceutical compositions may conveniently be presented in unit dosage form and may be prepared by any of the methods well-known in the art of pharmacy.

All methods may include the step of bringing the active agent into association with a carrier which constitutes one or more accessory ingredients. In general, compositions are prepared by uniformly and intimately bringing the active compound into association with a liquid carrier, a finely divided solid carrier, or both, and then, if necessary, shaping the product.

Compositions suitable for oral administration may be presented as discrete units, such as capsules, tablets, lozenges, each containing a predetermined amount of the active compound. Other examples of compositions include suspensions in aqueous liquids or non-aqueous liquids such as a syrup, elixir or an emulsion. Examples of compositions for parenteral administration include, without being limited to, sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Examples of aqueous carriers are water, alcoholic/aqueous solutions, emulsions or suspensions, for example saline and buffered media. Examples of parenteral vehicles are sodium chloride solution, Ringer's dextrose, dextrose and sodium chloride, and lactated Ringer's or fixed oils. Examples for intravenous vehicles are fluid and nutrient replenishers, electrolyte replenishers (such as those based on Ringer's dextrose), and the like. Preservatives and other additives may also be present such as, for example, antimicrobials, anti-oxidants, chelating agents, and inert gases, and the like.

The pharmaceutical agents of some embodiments of the invention may be administered alone, in combination with each other, and/or in combination with other drug therapies and/or treatments. Examples of therapies and/or treatments may include, but are not limited to: surgical intervention, chemotherapy, radiotherapy, and adjuvant systemic therapies.

In some embodiments, the invention also provides one or more kits comprising one or more containers comprising one or more of the compounds or agents of the invention. Additional materials may be included in any or all kits of the invention, and such materials may include, but are not limited to, for example, buffers, water, enzymes, tubes, control molecules, etc. One or more kits may also include instructions for the use of the one or more compounds or agents of the invention for the diagnosis and/or treatment of a disease or condition.

Any means for the introduction of polynucleotides into mammals, human or non-human, or cells thereof may be adapted to the practice of this invention for the delivery of the various nucleic acids, or derivatives thereof, of the invention into cells. These methods may be adapted to deliver any nucleic acid as provided by this invention in vitro, ex vivo, or in vivo, for example into cells in culture, cells in explanted tissues or cells in the body of a subject. In one embodiment of the invention, nucleic acids are delivered to cells by transfection, i.e., by delivery of “naked” nucleic acids or in a complex with a colloidal dispersion system. A colloidal system includes macromolecule complexes, nanocapsules, microspheres, beads, and lipid-based systems including oil-in-water emulsions, micelles, mixed micelles, and liposomes. The preferred colloidal system of this invention is a lipid-complexed or liposome-formulated nucleic acids. Formulation of nucleic acids, e.g. with various lipid or liposome materials, may be effected using known methods and materials and delivered to the recipient mammal. See, e.g., Canonico et al, Am J Respir Cell Mol Biol 10:24-29, 1994; Tsan et al, Am J Physiol 268; Alton et al., Nat. Genet. 5:135-142, 1993 and U.S. Pat. No. 5,679,647 by Carson et al.

Nucleic acids according to this invention can be delivered to cells using viral vectors. The nucleic acids provided by this invention may be incorporated into any of a variety of viral vectors useful in gene therapy, such as recombinant retroviruses, adenovirus, adeno-associated virus (AAV), and herpes simplex virus-1, or recombinant bacterial or eukaryotic plasmids. The incorporation of nucleic acids into such vectors and the generation of viral particles and their administration are well known to those skilled in the art.

The function and advantage of these and other embodiments of the present invention will be more fully understood from the examples below. The following examples are intended to illustrate the benefits of the present invention, but do not exemplify the full scope of the invention.

### EXAMPLES

### Materials and Methods

### Definitions of Acronyms and Abbreviations

bp: base pair
IGS: internal guide sequence
RBS: ribosome binding site
CDS: coding sequence
GFP: green fluorescent protein
MUG: 4-methylumbelliferyl beta-D-galactopyranoside
nt(s): nucleotide(s)
PCR: polymerase chain reaction
IPTG: isopropyl β-D-1-thiogalactopyranoside

The Tetrahymena ribozyme genomic sequence can be found, for example, under GenBank accession number V01416 in the NCBI nucleotide database:

>gi|10840|emb|V01416.1|Fragments of a
Tetrahymena gene for 26s rRNA (with intron)
(SEQ ID NO: 1)
TGACGCAATTCAACCAAGCGCGGGTAAACGGCGGGAGTAACTATGACT
CTCTAAATAGCAATATTTACCTTTGGAGGGAAAAGTTATCAGGCATGC
ACCTGGTAGCTAGTCTTTAAACCAATAGATTGCATCGGTTTAAAAGGC
AAGACCGTCAAATTGCGGGAAAGGGGTCAACAGCCGTTCAGTACCAAG
TCTCAGGGGAAACTTTGAGATGGCCTTGCAAAGGGTATGGTAATAAGC
TGACGGACATGGTCCTAACCACGCAGCCAAGTCCTAAGTCAACAGATC
TTCTGTTGATATGGATGCAGTTCACAGACTAAATGTCGGTCGGGGAAG
ATGTATTCTTCTCATAAGATATAGTCGGACCTCTCCTTAATGGGAGCT
AGCGGATGAAGTGATGCAACACTGGAGCCGCTGGGAACTAATTTGTAT
GCGAAAGTATATTGATTAGTTTTGGAGTACTCGTAAGGTAGCCAAATG
CCTCGTCATCTAATTAGTGACGCGCATGAATGGATTA

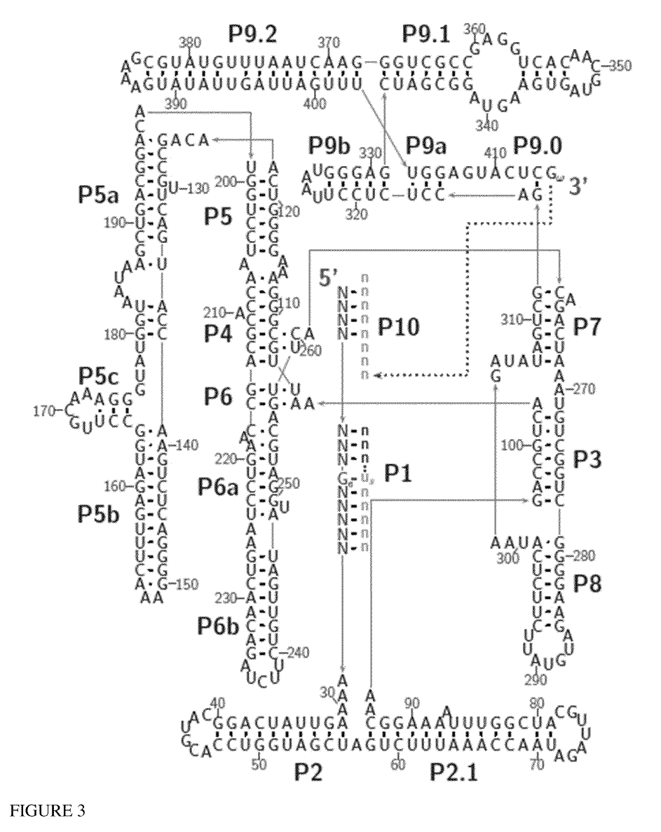

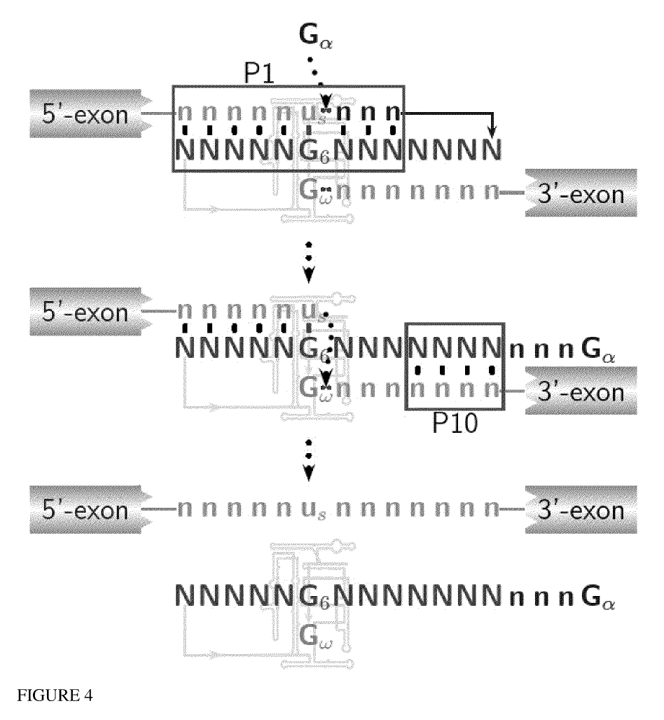

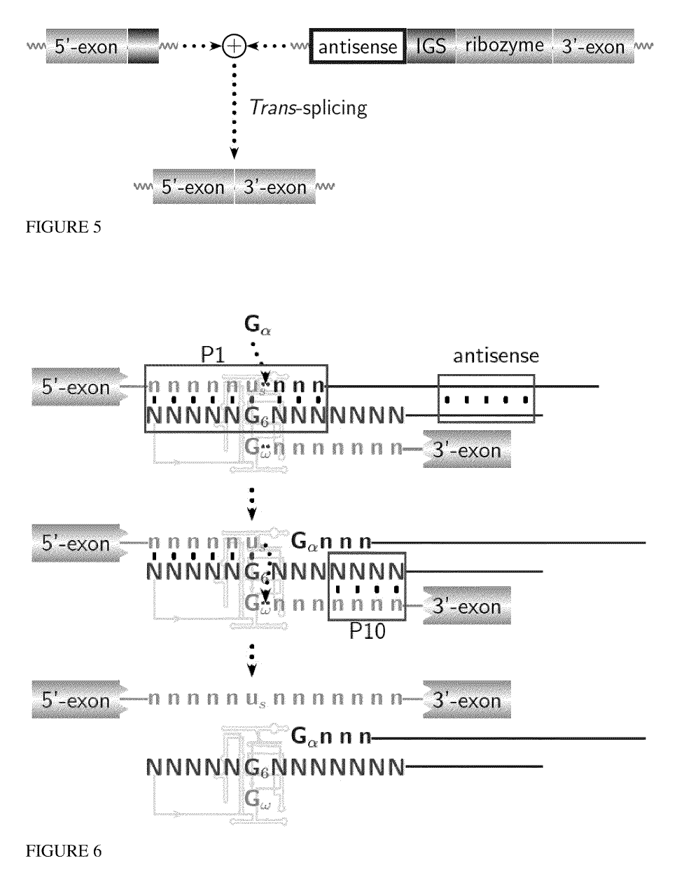

References in the example section to “the ribozyme”, unless modified by additional descriptive matter, indicate the Tetrahymena ribozyme sequence from 28-414 (FIG. 3).

Shown below is the Tetrahymena ribozyme sequence from 28-414 depicted in FIG. 3, with an IGS at the 5′ end (bold).

(SEQ ID NO: 2)
NNNNNNNG₆NNNNNAAAAGUUAUCAGGCAUGCACCUGGUAGCUAGUCU
UUAAACCAAUAGAUUGCAUCGGUUUAAAAGGCAAGACCGUCAAAUUGC
GGGAAAGGGGUCAACAGCCGUUCAGUACCAAGUCUCAGGGGAAACUUU
GAGAUGGCCUUGCAAAGGGUAUGGUAAUAAGCUGACGGACAUGGUCCU
AACCACGCAGCCAAGUCCUAAGUCAACAGAUCUUCUGUUGAUAUGGAU
GCAGUUCACAGACUAAAUGUCGGUCGGGGAAGAUGUAUUCUUCUCAUA
AGAUAUAGUCGGACCUCUCCUUAAUGGGAGCUAGCGGAUGAAGUGAUG
CAACACUGGAGCCGCUGGGAACUAAUUUGUAUGCGAAAGUAUAUUGAU
UAGUUUUGGAGUACUCG_{ω}

All nucleotides are numbered based on the native Tetrahymena ribozyme. The nucleotides in the IGS are numbered from −13 to −1 with −13 being the 5′−most base and −1 being immediately upstream of the ribozyme. In figures, the gray outline of the ribozyme is symbolic for the ribozyme core. Scissile phosphodiester bonds are indicated by dots or dotted lines. “G264A” is the ribozyme with a single point mutation of the G at base 264 in the guanosine binding site. The G264A mutant shows no change in the folded state but is incapable of binding guanosine [96, 104]. Thus, the G264A ribozyme cannot splice and is used as a negative control in many experiments. A couple special bases involved in splicing are specially labeled.

G_{α}: exogenous G added in first step

G_{ω}: last G in ribozyme (nt 414)

G₆: G at −6 in the IGS (nt 22) that forms the critical G:U

uₛ: target U splice point

### Data Analysis

Unless otherwise stated, all error bars on graphs and ±values in tables indicate the standard error of the mean using at least four colonies from measurements done on at least two different days.

### Biobrick Parts

Several parts from the Registry of Standard Biological Parts (http://partsregistry.org) were reused (sequence and details available in the registry): BBa_B0015: transcriptional terminator BBa_B0034: strong ribosome binding site (RBS) BBa_F2620: promoter inducible to by acyl-homoserine lactone (AHL) [29] BBa_R0040: without TetR, as used for all constructs described herein, functions as a strong constitutive promoter

The “BBa” prefix is dropped to conserve space in diagrams. All part combinations with BioBrick parts have an implied mixed site sequence (TACTAGAG) between the parts. The sequence TACTAG is present between an RBS and the start codon of a coding sequence.

### GFP

The GFP variant (BBa_E0043) used for most experiments was derived from an untagged gfpmut3* (BBa_E0040) [4]. Relative to BBa_E0040, BBa_E0043 contains 6 base mutations that change amino acids 64 and 65 plus one silent mutation in amino acid 63 to codon optimize it for E. coli. From wild-type GFP, BBa_E0043 has the mutations S2R, F64L, S65T, and S72A.

### Plasmids

All constructs were cloned into one of the following BioBrick vectors [138].

pSB1A3: pSB1A3 is derived from pUC19 with a high copy pMB1 origin and an ampicillin resistance gene.
pSB2K4: I constructed pSB2K4 via site-directed mutagenesis of pSB2K3 to remove restriction sites. pSB2K4 contains two origins including one which is LacI-regulated. In the repressed state, pSB2K4 is at low copy and when induced, the plasmid shifts to high copy. All measurements with constructs on pSB2K4 had 1 mM IPTG added to induce high copy number. Cells with pSB2K4 are kanamycin resistant.
pSB3K3: pSB3K3 contains a low copy p15A origin with a kanamycin resistance gene.
pSB4C5 and pSB4K5: These plasmids contain a low copy pSC101 origin. pSB4C5 confers chloramphenicol resistance and pSB4K5 confers kanamycin resistance.

### Cloning and Growth Conditions

All cloning steps followed standard molecular biology protocols. Most constructs were assembled using PCR and restriction enzyme techniques. Some longer sequences were synthesized by Integrated DNA Technologies (IDT). All constructs were transformed into Top10 (DH10B) E. coli and verified by sequencing. Although all measurements were done using Top10, recent results show that DH10B has a high overall mutation rate due to insertion sequence transposition events [43]. This genetic instability could explain some results where unusually variable measurement data became more reliable after a DNA miniprep and re-transformation back into fresh Top10.

For measurements, I grew cells in Neidhardt EZ rich defined media (Teknova) [110] to increase reproducibility and to decrease background fluorescence. All growth was at 37° C. with shaking. Cells were either grown in individual tubes or in 96-well deep plates covered with a 3M Micropore breathable membrane to provide oxygen during growth.

### Plate Reader

All absorbance and fluorescence measurements were made using a Wallac Victor3 96-well plate reader (Perkin Elmer, Waltham, Mass.). The measured A600 is linearly related to OD600, with one A600 unit roughly equal to three OD units (http://openwetware.org/index.php?title=Endy:Victor3_absorbance_labels&oldid=174399). Excitation/emission filters of 488/535 nm and 570/620 nm were used to measure GFP and mCherry. Using purified EGFP (BioVision #4999-100), the detection limit of the plate reader was calculated at about 19 ·10⁹ molecules of EGFP per well.

### GFP Growth Measurement

To measure GFP during growth, cells were inoculated into EZ media in a 96-well plate. The plate reader was temperature controlled at 37° C. After an initial 10 s shake, the following cycle was used to grow, aerate, and measure the cells overnight:

1. Shake 15 s

2. Measure A600 absorbance

3. Measure GFP fluorescence

4. Dispense 5 μl of water into all wells to counteract evaporation

5. Wait 270 s

6. Shake 15 s

7. Measure A600 absorbance

8. Measure GFP fluorescence

9. Wait 270 s

From the absorbance and fluorescence measurements, I estimated the maximum GFP synthesis rate per cell. At each time point, the fluorescence/A600 ratio is an estimate of the number of GFP molecules per cell. The GFP synthesis rate is given by the change in fluorescence/A600 over time. For any time point i, the GFP synthesis rate is calculated as follows. For each time point j>i, a regression line is fit using the fluorescence/A600 and time points between i and j. The slope of the regression line with the highest R² over all j is the GFP synthesis rate for time i. I used the maximum GFP synthesis rate over all i as the quantitative indicator of GFP expression. Empirically, using the maximum slope of the best fit regression lines showed less variation between multiple runs than other methods. As GFP is quite stable and does not degrade [4], the GFP synthesis rate should be proportional to the gfp RNA levels.

### Fluorescence Measurement

I made single time point fluorescence readings for GFP or mCherry by growing colonies overnight. After overnight growth, 200 μl of the culture was transferred to a 96-well plate and the A600 and fluorescence measured. I used the fluorescence/A600 ratio as an estimate of the number of fluorescent molecules per cell.

### Laczα Activity Measurement

Top10 cells contain the LacZω fragment to complement LacZα in forming active LacZ (β-galactosidase). To measure LacZ activity, and hence the amount of LacZα, I used the fluorescent substrate 4-methylumbelliferyl-β-D-galactopyranoside (MUG). MUG (Sigma Aldrich #M1633) was dissolved in DMSO at a concentration of 2 mg/ml and used as a 10× stock solution. I grew colonies overnight in EZ media with 1 mM IPTG. 200 μl of the culture was transferred to a 96-well plate and the A600 absorbance measured. Then, some amount of the culture (e.g., 10 μl) was transferred to a new well containing 20 μl of the stock MUG solution and PBS up to a final volume of 200 μl (e.g., 170 μl). The plate reader was used to measure fluorescence using excitation/emission filters of 355/460 nm with 30 s delay between reads. The plate temperature was set at 30° C.

From the MUG fluorescence data, regression lines were fit using all points from time zero to each possible end point. The line with the highest R² was used to estimate LacZ activity. If the best R² was less than 0.9, the activity was set to zero. Otherwise, the raw LacZ activity was set to the slope of the line with the highest R². The raw activity was normalized by the A600 and the volume of the culture used. LacZ standard reference Samples with low LacZ expression were normalized using an absolute standard reference of purified β-galactosidase (Sigma Aldrich #G4155) at a concentration of 1.7 mg/ml (according to supplier). The stock solution was diluted 1000× into 50% glycerol to give a working solution of 1.7 ng/μl. A standard curve was generated from this diluted stock, using a tetramer molecular weight of 465 kDa. A regression line through the origin for the reference standard was used to convert raw LacZ activity to an equivalent number of LacZ molecules.

For each sample, the raw LacZ activity was calculated, converted to equivalent number of LacZ molecules, and then normalized by the A600 and volume used (usually 180 μl). Thus, the LacZ activity is in units of equivalent LacZ molecules per absorbance unit per 111. The detection limit on the plate reader for a reaction run approximately 3 hours is 3 ·107 molecules of equivalent LacZ. Using the standard protocol with 180 μl of overnight culture and with a typical saturating A600 of around 1, the lower limit of normalized LacZ activity is around 2 ·10⁵. Assuming saturated cultures have 10⁶ cells per μl, the plate reader can detect less than one LacZ molecule per cell. However, the reference standard is full length LacZ, whereas all constructs use LacZα complementation, which is only about 24% as active as the full length LacZ [169]. Thus, for LacZα complementation, the number of LacZα molecules required for detection is larger.

### Example 1

### IGS Design

In the ribozyme's native context, the internal guide sequence (IGS) is a 13 nt sequence that forms the P1 and P10 guiding helices. The P1 helix is formed in the first step of splicing and determines the 5′-splice point. The P10 helix is formed in the second step of splicing and helps in aligning the 3′-exon. Thus, the IGS is the primary interface between the ribozyme and the exons. There are no known sequence requirements for the exons other than the us at the 5′-splice site.

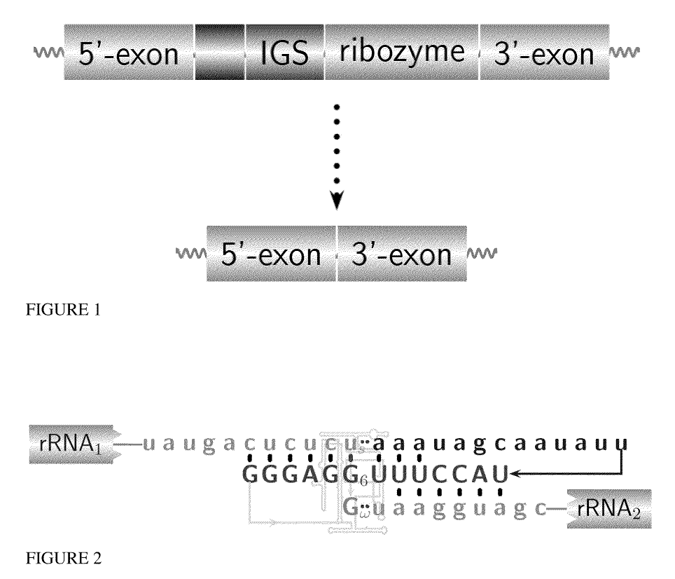

For splicing in a new context, the IGS needs to be changed to match the 5′- and 3′-exons. Although selection protocols can find an efficient IGS [27, 49, 61], it is preferable to have rules for rationally designing an IGS without experimentation. For some applications, we may also want to tune splicing efficiencies by changing the IGS, similar to how we can tune promoter and RBS strengths. In the native context (FIG. 2), part of the P1 and P10 helices overlap. An overlapping P1 and P10 would require that the 5′ and 3′ exon sequences be coordinated. To simplify engineering, we can eliminate the P1 and P10 overlap by shortening the P10 to 4 by and keeping the P1 at 9 bp. The P10 is not as critical for efficient splicing and it has been shown that 4 by is enough [89].

It is not clear that the strongest possible pairing in P1 and P10 would lead to the most efficient splicing. The “Goldilocks principle” applies to many things in biology. Interactions should not be too strong and should not be too weak. They should be just right. Strong base pairing could inhibit splicing as the ribozyme needs to make and break base pairs during the process of splicing. For example, a strong P1 base pairing could compete with formation of the P10 pairing, lowering splicing efficiency [61]. Also, if the ribozyme does not dissociate quickly from the spliced product, the ribozyme could possibly cleave the spliced product, leading to disconnected exons.

I sought to test how the strength of the IGS pairing impacts splicing efficiency. A rationally designed IGS containing 12 Watson-Crick base pairs and one G₆:uₛ wobble base pair was expected to have strong interactions. From this IGS, all single point mutants were constructed and characterized. I expected the mutations to weaken the interaction strength. Using the experimental data, I developed a model that used computational RNA folding to estimate splicing efficiency.

### Experimental Setup

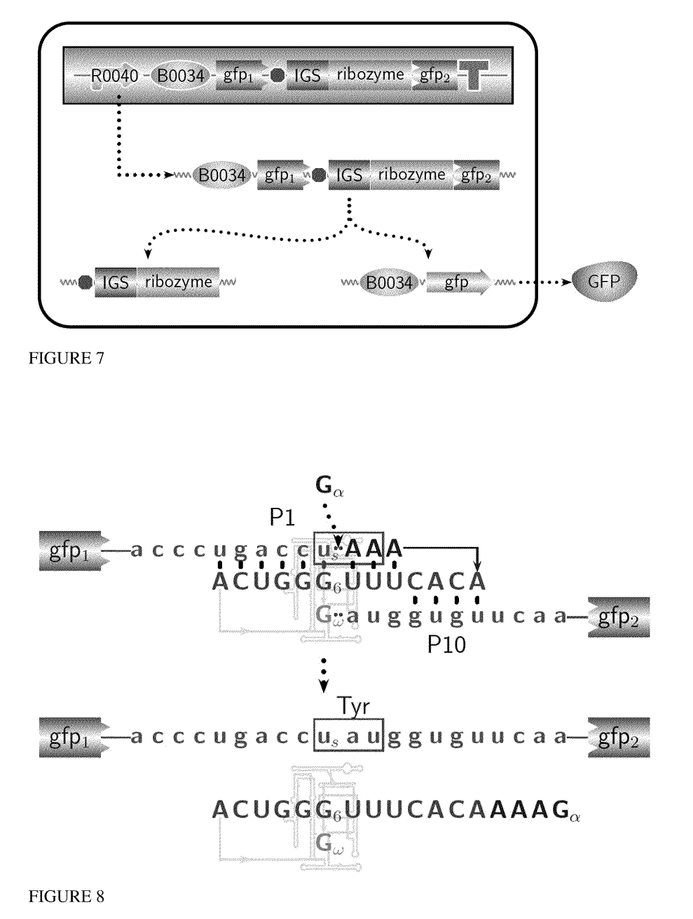

I used a cis-splicing GFP construct to characterize splicing activity (FIG. 7). To eliminate any possible background fluorescence from non-spliced constructs, the codon for the fluorophore of GFP (Tyr66) [152] was split (FIG. 8). The pre-spliced RNA contained a stop codon in the 5′-exon, allowing only half of GFP to be produced. In addition, even if the 3′-half of GFP were somehow translated, the tyrosine fluorophore would not be made. This design prevents two GFP peptide fragments from coming together and complementing each other [26].

Based on the rationally designed IGS0 shown in FIG. 8, IGS variants were made via ligation of oligos and cloned into the plasmid pSB2K4. Most of the construction and sample processing were done in 96-well plates. After transformation, as an alternative to picking colonies from agar plates, single colonies were obtained by using serial dilutions on a robot. Variants were sequenced with at least one primer that covered the mutated IGS region. Not all variants were completely sequenced, so a mutation near the end of GFP could potentially exist in the variant library. However, no clones had mutations outside of the IGS region, even the clones that were completely sequenced. After sequencing, initial fluorescence tests showed high variability between runs, perhaps due to non-clonal populations or genomic mutations. All constructs were miniprepped and re-transformed simultaneously into the same batch of competent cells. Constructs from this re-transformation showed reliable results and were used for all data measurements.

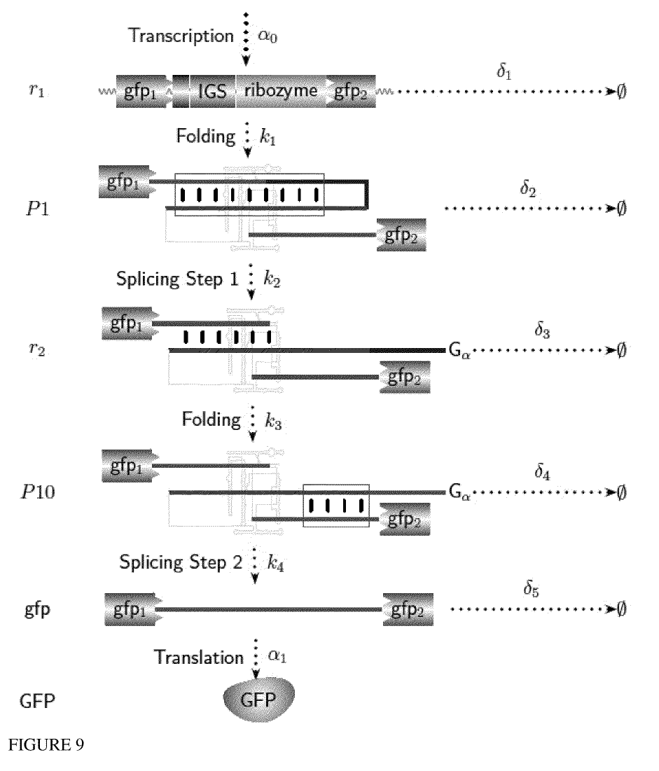

TABLE 1
The differential equations on the left represent the splicing model in
FIG. 9. On the right is the transcription and translation rates
for a reference intact GFP not containing a ribozyme.

| Splicing GFP | Intact GFP |
|---|---|
| $\frac{d [ GFP ]}{dt} = {α}_{1} [ gfp ]$ | $\frac{d [ GFP ]}{dt} = {α}_{1} [ gfp ]$ |
| $\frac{d [ gfp ]}{dt} = {k}_{4} [ P 10 ] - {δ}_{5} [ gfp ]$ | $\frac{d [ gfp ]}{dt} = {α}_{0} - {δ}_{5} [ gfp ]$ |
| $\frac{d [ P 10 ]}{dt} = {k}_{3} [ {r}_{2} ] - ( {k}_{4} + {δ}_{4} ) [ P 10 ]$ | |
| $\frac{d [ {r}_{2} ]}{dt} = {k}_{2} [ P 1 ] - ( {k}_{3} + {δ}_{3} ) [ {r}_{2} ]$ | |
| $\frac{d [ P 1 ]}{dt} = {k}_{1} [ {r}_{1} ] - ( {k}_{2} + {δ}_{2} ) [ P 1 ]$ | |
| $\frac{d [ {r}_{1} ]}{dt} = {α}_{0} - ( {k}_{1} + {δ}_{1} ) [ {r}_{1} ]$ | |

Fluorescence measurements were made by inoculating from glycerol stocks into 500 μl LB media in a deep 96-well plate. After overnight growth, 5 μl was used to inoculate 200 μl EZ media with 1 mM IPTG. The IPTG induced the plasmid copy number of pSB2K4 to be high. The cells were grown on the plate reader. Each run had two plate replicates that were averaged. The mean and standard error were calculated from six independent runs.

### Splicing Model

FIG. 9 shows an ordinary differential equation model of the splicing process. There are two folding and two splicing steps to go from the unspliced RNA to the spliced gfp RNA. The model assumes that all steps are irreversible and that the GFP protein is extremely stable [4]. Each step contains a forward rate and a general degradation rate (δₙ). The δₙ terms include both RNA degradation and incorrect side reactions, such as inaccurate splicing or misfolding into kinetic traps [79, 114]. Table 1 contains the differential equations derived from the model. A reference intact GFP, not containing a ribozyme and expressed using the same promoter and RBS, was modeled using the same rates.

To estimate the P1 folding rate, I used the Vienna RNA package [59] to calculate EP1, the ensemble free energy for the P1 folding. The P1 sequence acuugucacuacccugaccuAAA(IGS)AAA was folded where (IGS) represents the specific IGS sequence being tested. The lowercase nucleotides come from the first half of GFP and the three As after the IGS come from the beginning of the ribozyme. To estimate the other three rates, I used the kinefold program to do kinetic folding [165]. I only used the last time point from the folding simulations and averaged the results from five runs. To simulate the first step of splicing, the P1 sequence above was folded for 8 simulated seconds, which is the time expected for transcription of the ribozyme. The sequence was folded co-transcriptionally with a new nucleotide added every 20 ms.

I calculated the probability Pr(G₆:uₛ) for the G:U pairing at the splice site from the simulation. Similarly, I calculated Pr(G₆:uₒₜₕₑᵣ), the probability of G₆ pairing with a different U. For an IGS without a G at position −6, probabilities of zero were used. To simulate the second step of splicing, a renaturation fold was performed on the sequence acuugucacuacccugaccuXXXXXGAAA(IGS)AAALXXXXXXXXXXaugguguuca for 5 simulated seconds. An X is treated by kinefold as a special base that never base pairs and was inserted as a spacer. The L tells kinefold to fold the two halves separately for the first third of the time and to allow the halves to fold together in the remaining time. In the second splicing step, the IGS potentially pairs with both the 5′- and 3′-exons. Pr(IGS:3′-exon), the probability of the P10 helix forming, was calculated assuming that any base pair between the IGS and the 3′-exon is sufficient for the P10 helix. Some IGS variants had multiple helices with pairing between the IGS and the 3′-exon. For these variants, the probabilities were summed and capped at 1, which is an upper bound on the pairing probability. Pr(IGS:uₛ), the probability of the splice site U base pairing with any nucleotide in the IGS, estimates the likelihood of the 5′-exon maintaining its pairing with the IGS.

### Experimental Data

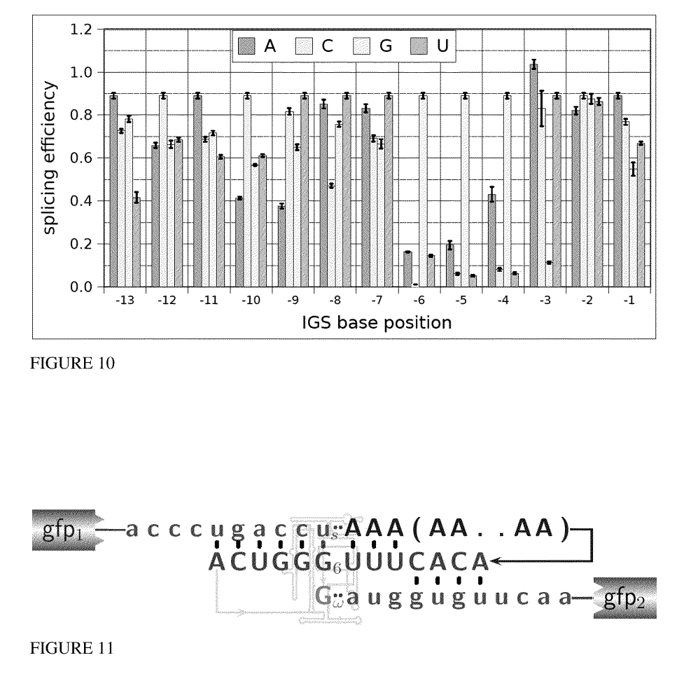

FIG. 8 shows the rationally designed 13 nt IGS₀. Relative to IGS₀, all single base mutations were made, for 39 variants in addition to IGS₀. IGS^{N}ₓ is the mutant with base N at position −x. I estimated the splicing efficiency using the maximum GFP synthesis rate normalized to intact GFP. FIG. 10 shows the splicing efficiencies for the IGS variants. At each of the 13 positions, one of the bars is IGS₀ and the other 3 are the single point mutations at that position. IGS₀ showed almost 90% of the activity of intact GFP, indicating efficient splicing. The single mutants showed a large range of efficiencies. A G264A inactive ribozyme control with IGS₀ was run simultaneously and had a splicing efficiency of 0.003, which is an order of magnitude lower than the lowest of the IGS variants. In addition to the single point mutants, three double mutants were characterized: IGS^{G}₁+IGS^{T}₁₀, IGS^{G}₃+IGS^{A}₁₂, and IGS^{G}₁+IGS^{A}₁₂.

Table 2 shows the measured splicing efficiencies for the double mutants and the single mutants as reference. The results indicate that the effects of multiple mutations are not additive, with double mutations sometimes being more efficient than either of the single mutations. In the native ribozyme, a 14 nt unpaired loop region is between the IGS and the 5′-exon. Although all of the single point mutants were constructed with no loop region, additional spacer sequence could possibly increase splicing efficiency by reducing the steric hindrance for P1 formation. To test this possibility, the spacing between the IGS and the 5′-exon was varied from 0-30 nucleotides. I chose a poly-A spacer region to minimize the probability of additional base pairings (FIG. 11). All spacers used the reference IGS0. Table 2 shows the splicing efficiencies of spacers containing 10, 20, and 30 As. A clear trend is seen with longer spacers corresponding to lower efficiencies.

TABLE 2
The splicing efficiencies for several additional IGS variants are shown.

| IGS | Splicing efficiency | Spacer | Splicing efficiency |
|---|---|---|---|
| IGS₁^{G} | 0.549 ± 0.031 | 0 | 0.890 ± 0.014 |
| IGS₃^{G} | 0.114 ± 0.005 | A¹⁰ | 0.799 ± 0.010 |
| IGS₁₀^{T} | 0.611 ± 0.007 | A²⁰ | 0.753 ± 0.011 |
| IGS₁₂^{A} | 0.659 ± 0.012 | A³⁰ | 0.686 ± 0.009 |
| IGS₁^{G} + IGS₁₀^{T} | 0.692 ± 0.012 | | |
| IGS₃^{G} + IGS₁₂^{A} | 0.466 ± 0.013 | | |
| IGS₁^{G} + IGS₁₂^{A} | 0.688 ± 0.013 | | |

The left table shows three double mutants, with the single mutations shown as reference.
The right table shows the effect of changing the spacer length.
Aⁿ indicates a spacer containing n additional
As inserted between the 5′-exon and the IGS.
IGS₀ and all the single and double mutants have a spacer length of 0.

### Fitting a Model Using the Data

Using the equations in Table 1 and assuming a quasi-steady state for all RNA species, the GFP synthesis rate for the reference GFP is _{—}0_{—}1=_{—}5 and the GFP synthesis rate for the splicing GFP is given by

$$\frac{d [ GFP ]}{d t} = \frac{{α}_{0} {α}_{1}}{{δ}_{5}} \frac{{k}_{1}}{( {k}_{1} + {δ}_{1} )} \frac{{k}_{2}}{( {k}_{2} + {δ}_{2} )} \frac{{k}_{3}}{( {k}_{3} + {δ}_{3} )} \frac{{k}_{4}}{( {k}_{4} + {δ}_{4} )} . \qquad ( Equation 1 )$$

The splicing efficiency is defined by the relative synthesis rate

$$splicing efficency = relative \frac{d [ GFP ]}{d t} = {s}_{1} {s}_{2} {s}_{3} {s}_{4} , \\ where \\ {s}_{n} = \frac{{k}_{n}}{{k}_{n} + {δ}_{n}} . \qquad ( Equation 2 )$$

sₙ, is the relative efficiency for step n. The overall splicing efficiency is the product of the efficiencies at each step. The efficiency at each step depends on the relative rate of the forward reaction, versus the rate of non-productive reactions, δₙ. For each step, δₙ includes a constant rate that that is assumed independent of the IGS, such as the RNA degradation rate. All other rates can be normalized to δₙ n so I let δ⁰ₙ=1. It is desirable to computationally predict the splicing efficiency from sequence using RNA folding algorithms. For each IGS variant, E_{P1}, Pr(G₆:uₛ), Pr(G₆:uₒₜₕₑᵣ), Pr(IGS:3′-exon), and Pr(IGS:uₛ) were computationally determined using RNA folding as described elsewhere herein. Some of the calculated probabilities were zero, even though all IGS variants showed detectable activity. To correct for low probabilities or limitations in the folding algorithm, all probabilities less than a cutoff threshold were instead set to the cutoff probability. The following equations map computational RNA folding data into kinetic rates and splicing efficiency:

$$\begin{matrix}\begin{matrix}{s}_{1} \vert = \frac{{k}_{1}}{{k}_{1} + {δ}_{1}} \\ = \frac{{k}_{1}}{{k}_{1} + {δ}_{1}^{0}} \\ = \frac{{f}_{1} {e}^{- {E}_{p 1} / ( kT )}}{{f}_{1} {e}^{- {E}_{p 1} / ( kT )} + 1}\end{matrix} & ( Equation 3 ) \\ \begin{matrix}{s}_{2} = \frac{{k}_{2}}{{k}_{2} + {δ}_{2}} \\ = \frac{{k}_{2}}{{k}_{2} + {{k}^{′}}_{2} + {δ}_{2}^{0}} \\ = \frac{{f}_{2} \cdot max ( Pr ( {G}_{6} : {u}_{s} ) , {l}_{2} )}{\begin{matrix}{f}_{2} \cdot max ( Pr ( {G}_{6} : {u}_{s} ) , {l}_{2} ) + \\ {f}_{2} \cdot max ( Pr ( {G}_{6} : {u}_{other} ) , {l}_{2} ) + 1\end{matrix}}\end{matrix} & ( Equation 4 ) \\ \begin{matrix}{s}_{3} = \frac{{k}_{3}}{{k}_{3} + {δ}_{3}} \\ = \frac{{k}_{3}}{{k}_{3} + {δ}_{3}^{0}} \\ = \frac{{f}_{3} \cdot max ( Pr ( IGS : {3}^{′} - exon ) , {l}_{3} )}{{f}_{3} \cdot max ( Pr ( IGS : {3}^{′} - exon ) , {l}_{3} ) + 1}\end{matrix} & ( Equation 5 ) \\ \begin{matrix}{s}_{4} = \frac{{k}_{4}}{{k}_{4} + {δ}_{4}} \\ = \frac{{k}_{4}}{{k}_{4} + {δ}_{4}^{0}} \\ = \frac{{f}_{4} \cdot max ( Pr ( IGS : {u}_{s} ) , {l}_{4} )}{{f}_{4} \cdot max ( Pr ( IGS : {u}_{s} ) , {l}_{4} ) + 1}\end{matrix} & ( Equation 6 )\end{matrix}$$

The seven free parameters (four fₙ, and three lₙ) were fit to the experimental data for IGS₀ and the 39 single point mutations, using the Levenberg-Marquardt algorithm, with initial values of one for the fn parameters and zero for the ln parameters. To assess the contribution of each step, fits were done with all combinations of the four steps. Table 3 shows the R² values for different fits. Parameter values were similar across the different fits, indicating a robust fitting procedure. Analysis of the fit parameters led to two changes in the model.

TABLE 3
Splicing was modeled using different combinations of the four steps
in the model (s₁, s₂, s₃, s₄).
The experimental data in FIG. 10 was fit using these subsets of the
full model and the correlation R² between the experimental
and model data is shown.

| | | | | | | | |
|---|---|---|---|---|---|---|---|
| s₁ | 0.292 | s₁, s₂ | 0.650 | s₁, s₂, s₃ | 0.744 | s₁, s₂, s₃, s₄ | 0.744 |
| s₂ | 0.601 | s₁, s₃ | 0.010 | s₁, s₃, s₄ | 0.001 | | |
| s₃ | 0.004 | s₁, s₄ | 0.344 | s₁, s₂, s₄ | 0.635 | | |
| s₄ | 0.202 | s₂, s₃ | 0.721 | s₂, s₃, s₄ | 0.721 | | |
| | | s₂, s₄ | 0.617 | | | | |
| | | s₃, s₄ | 0.414 | | | | |

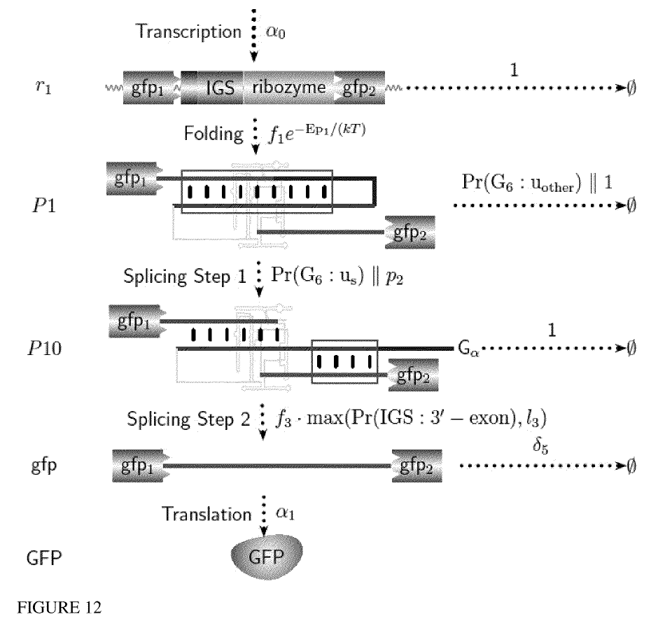

First, step 4 did not improve the model. The two parameters for step 4, f₄ and l₄, had values such that s₄=1 for all IGS variants. Therefore, step 4 was dropped from the model. Second, the fit for step 2 had a large f₂=10¹¹ and small l₂=10⁻¹², indicating that k₂>>δ⁰₂. The step 2 efficiency can be split into two cases. When the probability of G₆:U is non-zero for some U, the degradation rate is negligible so the step 2 efficiency is the ratio r=(r+1), where r=Pr(G₆:uₛ)=Pr(G₆:uₒₜₕₑᵣ). When there is a zero probability for any G₆:U pairing, to then δ⁰₂=1 and there is a fixed basal splicing efficiency. A single parameter p₂ can substitute for f₂ and l₂ leading to an overall basal step 2 efficiency of p₂=(p₂+1). With these two changes, FIG. 12 shows the simplified three-step model containing only four free parameters.

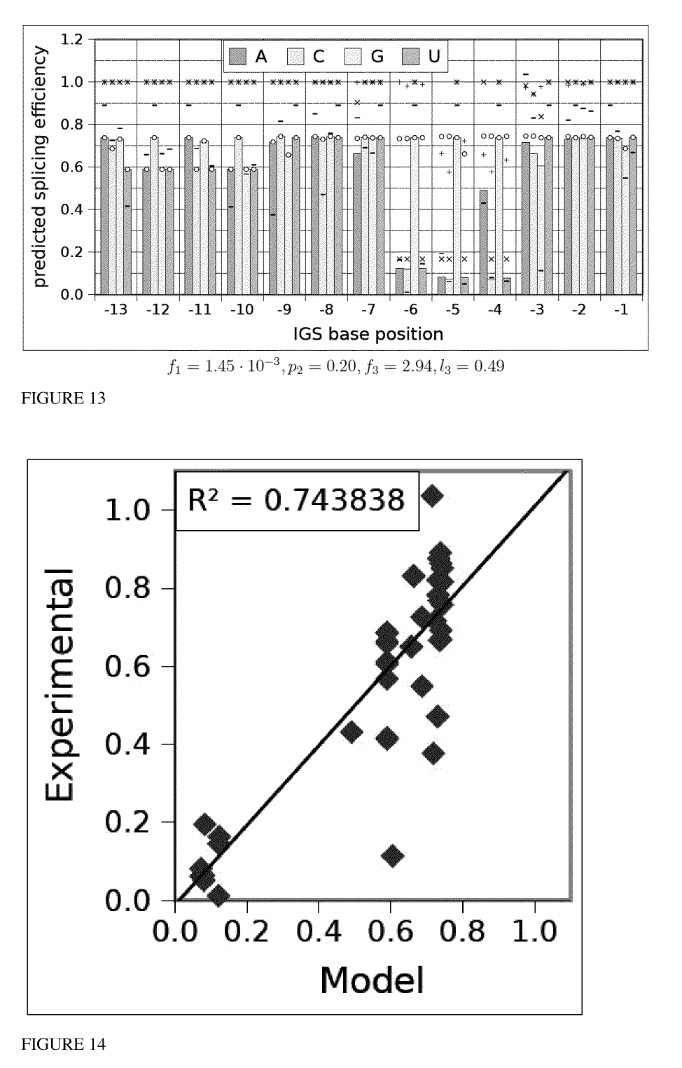

These four parameters were fit to the experimental data in FIG. 10. FIG. 13 shows the fit parameter values, the predicted splicing efficiencies, and the contribution from each step in the model. The correlation between the predicted and the experimental splicing efficiencies was 0.74 (FIG. 14). To test whether the model is likely indicative of folding reality, the experimental data was randomly shuffled among the IGS variants. The parameters were fit using the same three step model. For 25 random shuffles and fits, the R² values ranged from 0.01 to 0.19 with a median of 0.05.

As another cross-validation test, a random 25% of the data was left out for the parameter fitting and the R² was calculated over the entire data set. Over 25 such fits, the R² ranged from 0.71 to 0.75 with a median of 0.74. The fit parameter values did not change significantly using random subsets of the data. The low R² from the randomized data and nearly identical results using subsets of the data indicates that the model likely captures a real relationship between RNA folding and the experimental data without overfitting.

To test the model's generality and usefulness, I applied the model to the double mutants and spacer variants in Table 2. For the three double mutants, the relative ordering of efficiency was predicted correctly and had an R²=0.98. However, unlike the experimental data, the predicted efficiencies for the double mutants were less than the predicted efficiencies for the single mutants. For different length spacer sequences, the model predicted that the splicing efficiency would not vary significantly, contrary to the experimental results that showed a decrease in splicing efficiency with increasing spacer length.

### The IGS Determines Splicing Efficiency

Single mutations in the 13 nt IGS immediately upstream of the ribozyme can show a large range of splicing efficiencies (FIG. 10). The results suggest that we can tune the IGS for different splicing efficiencies. In addition to promoters and ribosome binding sites, the IGS provides another point between transcription and translation for controlling biological systems. All three IGS variants at position −3 had strange results. IGSA 3 was the only variant with a splicing efficiency greater than one, which is impossible in my model. IGSC 3 had the largest experimental variation of any of the samples, largely due to a single outlier data point. IGSG 3 showed the largest difference between the predicted efficiency and to experimental data. Overall, position −3 had a significantly larger error between the model and the data than other positions, indicating some unknown experimental or modeling error. I re-sequenced the entire plasmids of the three IGS3 variants and did not find any mutations.

The −3 mutants were the only three that showed multiple possible pairings between the IGS and the 3′-exon during the folding simulation. Thus, the probability P^{r}(IGS:3′-exon), calculated as the sum of probabilities, was an overestimate of the true probability and may be one source of model error. Excluding the anomalous IGS^{A}₃, IGS₀ showed the most efficient splicing. IGS₀ had a G:U at the splice site, 8 Watson-Crick base pairs with the 5′-exon and 4 Watson-Crick base pairs with the 3′-exon. Although, in this experimental context, a simple IGS design heuristic was sufficient for engineering high efficiency splicing, previous results have shown that a strong P1 helix can lower splicing efficiency, presumably by competing with the formation of the P10 helix [61]. Thus, a different context containing a high GC content may be inhibited by having too strong of a Watson-Crick base pairing.

Mutations at position −9 provide interesting information as it is at the boundary between the P1 and P10 pairing. In the natural Tetrahymena ribozyme, a 6 by P10 is formed with several bases of the IGS being shared between the P1 and P10. To separate the dependence of the 5′-exon sequence from the 3′-exon sequence, my IGS designs only included a 4 by P10. The mutation IGS^{C}₉ is expected to increase the P10 pairing and correspondingly reduce the P1 pairing. Supporting the usefulness of the P10 pairing, IGS^{C}₉ was more efficient than both IGS^{A}₉ and IGS^{G}₉. However, having a longer P1 (IGS₀) appears to dominate over a longer P10 (IGS^{C}₉). A similar trend can be seen at position −8. In the P10 helix, position −8 of the IGS would be base paired with a U. The results show that the best mutations are indeed IGS^{A}₈ followed by IGS^{G}₈, both of which can base pair with the U.

### Computationally Predicting Splicing Efficiency

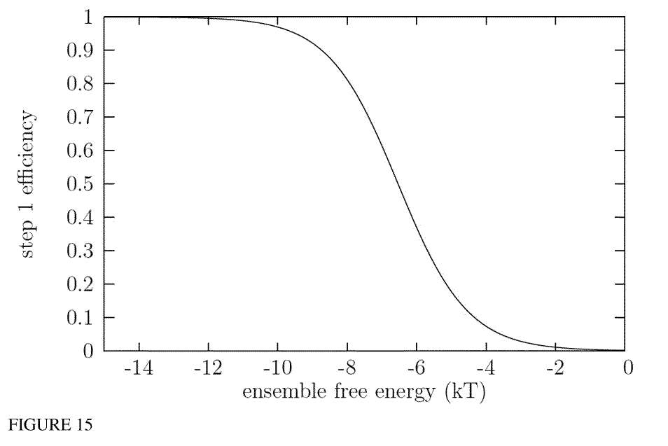

For tuning splicing efficiencies, it would be useful to predict the splicing efficiency from an IGS. Using a model for splicing (FIG. 12), I mapped data from RNA folding algorithms into kinetic parameters. The final model showed a correlation of R²=0.744 between the predicted and experimental splicing efficiencies, indicating that about 25% of the variation cannot be explained by the model. The model and fit parameters possibly provide insight into the splicing process. The fit parameters are qualitatively robust, and although the absolute numbers change slightly, the observations below appear to be valid for small changes in the data and methods of fitting. Step 1 of the model involves the folding of the P1 region in preparation for the first catalytic step. E_{P1}, the ensemble free energy from the folding of P1, was used to calculate the first step efficiency. The result of the fit, f₁=10⁻³, indicates that for an RNA with no folding energy, the side reactions are 1000 times faster than the forward reaction. With a folding energy of around 7 kT, the forward and side reactions are balanced (FIG. 15). Energies above 12 kT (7 kcal/mol at 37° C.) do not increase splicing efficiency. The IGS variants with a mutation at position −4 or −5 had the lowest free energy and are the most affected by this step (FIG. 13). The second step in the model is the first catalytic step of splicing. The initial model included a forward rate f₂, but the large value for the fit f₂ implies the forward catalytic rate must be fast relative to the competing reactions. Any pairing between G₆ and a U is assumed to splice equally efficiently. If the U is not the correct splice site, then the ribozyme will splice incorrectly. Thus, the efficiency depends simply on the probability ratio between the correct and incorrect splicing. If an incorrect G₆:U is highly unlikely, then any non-negligible probability for the correct G₆:uₛ pairing leads to a second step efficiency of 100%. If the G₆ has a calculated zero probability of pairing to any U, we would assume that the overall efficiency should be zero. Some variants, such as the IGS₆ variants, must by definition have a zero probability of G₆:U pairing. However, there was measurable splicing activity for all variants.

To reconcile the model with the data, a constant residual forward rate was added when there was no probability of any G₆:U pairing. The residual p₂ indicates the relative probability of correct splicing. The fit value indicates that there is a 20% chance of correct splicing and 80% of some other pathway, such as RNA degradation or incorrect splicing. Perhaps a small amount of correct splicing occurs independently of the IGS pairing or the folding algorithm is unable to compute low probabilities. As most variants have zero probability for an incorrect G₆:U pairing, step 2 in the model splits the variants into two main groups: one group that splices efficiently (100%) and one group that has some basal splicing amount (17%) (FIG. 13). Variants where the G₆ can base pair both with the correct and incorrect Us have intermediate efficiencies. For example, of all the variants, IGS^{G}₃ had the highest incorrect splicing probability with Pr(G₆:uₒₜₕₑᵣ)=14% and a corresponding Pr(G₆: uₛ)=70%, leading to an overall step 2 efficiency of 84%. The third step of the model is intended to include the conformation change and pairing required for the second splicing step. Whereas the second step in the model separates the highly efficient from the highly inefficient splicing constructs, the third step in the model leads to the small variations seen between IGS variants (FIG. 13). There are no known sequence requirements for the second step of splicing. As a first attempt at a model, I assumed that the second step depends on the IGS pairing with any part of the 3′-exon to form a “P10” helix. The forward rate was set equal to f₃·max(Pr(IGS:3′-exon), l₃). The fit value of f₃=2.9 is surprisingly small. Even for an ideal IGS, the forward rate would only be about three times faster than the non-productive rates, limiting the maximum splicing efficiency to 0.74. Thus, this parameter cannot be quantitatively correct as many of the variants have splicing efficiencies greater than 0.74. But compared with the first catalytic step, this parameter implies that the second catalytic step is more limiting for highly efficient splicing. The fit value for l₃=49% is surprisingly large. Three of the variants had calculated probabilities of IGS and 3′-exon pairing less than l₃.

Thus, l₃ is not due to a limitation of the folding program in calculating low probabilities. One interpretation is that a factor beyond pure thermodynamics helps the 3′-exon pair to IGS. For example, the ribozyme could facilitate alignment of the 3′-exon and accelerate its pairing with the IGS. Thus, splicing would be expected to occur even with a weak or non-existent P10 pairing, as has been found experimentally [154]. The original model contained a fourth step depending on the probability of the IGS pairing with us during the second splicing step. As the 5′-exon is not covalently linked during this step, if the 5′-exon were not also paired with the IGS, the exon could drift away from the ribozyme. Data fitting determined that this step provided no extra information. Perhaps the second step of splicing occurs quickly enough that the 5′-exon is still nearby for splicing. Alternatively, the 5′-exon could be released at a rate independent of the binding strength. The model assumes that dissociation of the spliced RNA from the ribozyme is fast or it can be lumped into another parameter.

Empirically, the exon dissociation energy did not fit well with the data. Although a stronger P1 or P10 pairing could lead to a slower dissociation rate, the data only indicates that stronger binding leads to more efficient splicing. However, in a different sequence context containing stronger binding energies, exon dissociation could become an important factor. Also, translation is ignored in the model. Translation of the spliced RNA could facilitate exon dissociation. Thus, the dissociation rate may be more important in non-translated splicing RNA systems.

### Model Limitations

Experimental error in the data would lead to an incorrect model. Even though there was low variability in the measured data, it is unclear whether fitting to fluorescence data is appropriate. Using a protein like GFP to measure RNA splicing is indirect and may have unseen problems. For example, a large fraction of GFP can go “dark” through misfolding and aggregating into inclusion bodies [73]. However, overall, the results fit reasonably well with the model, providing some confidence that an indirect measure of splicing may be sufficient.

The goal is to not only predict the splicing efficiency of one GFP splicing system, but rather to have a generalizable model that can apply to new systems. The model predicted correctly the splicing efficiency ordering for three double IGS mutants that were not used to fit the model, lending some support to the generality of the model. However, the model was not able to computationally predict the more radical sequence change of adding an extra spacer sequence between the IGS and the 5′-exon. The poly-A spacer may have had secondary effects on transcription or folding that were not included in the model.

Also, all the fit parameters were normalized to an assumed constant degradation rate. For the single point mutants here, it is safe to assume a constant rate across the samples. Different systems could have different degradation rates that would affect the overall balance between the forward and side reactions. It remains to be determined how sensitive splicing is to system-dependent side reactions. The G₆:U probabilities calculated for step 2 and the ensemble free energy calculated for step 1 are not independent. A weak P1 folding energy would lower the probability of all base pairs. However, a stronger overall P1 energy could either increase or decrease the G₆:uₛ probability, depending on whether the extra energy comes from a pairing including the G₆:uₛ. Although removing step 1 from the model does not significantly change the R², it qualitatively makes some visible changes.

For example, the major difference between IGS^{A}₄ and IGS₀ is in the free energy of the P1 region. For a general splicing model, it may make sense to eliminate this folding step. An accurate folding algorithm should take into account the folding energies when calculating the probabilities of G₆:U pairing. The model simplifies many aspects of splicing. For example, the first step of splicing occurs due to the destabilization of base pairs, especially the G₆:uₛ wobble base pair. A G₆:uₛ wobble base pair asymmetrically destabilizes base pairs on the 3′ side of the U [95]. Having a weaker base pair at position −7, such as the A:U base pair in the native sequence, may be important for high catalytic activity. This destabilization of the region 3′ to the us can help in transitioning to the second step of splicing. Biochemical details such as these are not handled by the model.

Another simplification is the requirement for the G:U base pair to be at position −6, as other positions can also splice [41, 119]. G:U at position −5 is slightly less efficient than at position −6, positions −4 and −7 are even less efficient, and splicing does not occur when the G:U is at positions −3, −8, or −9. In designing a new IGS that has a higher chance of splicing to accurately, we could make sure there are no Gs besides G₆ in the IGS. Also, alternative base pairings, such as U₄:As or G₆:Cs have been shown to splice [108]. Even though ribozyme activity is strongly correlated with it being in the folded state, the model does not explicitly include the ribozyme and how the surrounding sequence can affect the folding of the 384 nt ribozyme [88].

The IGS has been shown to affect ribozyme folding and the details of how this interaction works is unclear [114]. In addition, several ribozyme nucleotides including A114, A115, A301, and A302 are believed to form tertiary interactions with the IGS [97]. However, ignoring the entire ribozyme sequence is necessary given current computational constraints for folding long sequences and the lack of understanding of how ribozyme folding affects activity. Other than ignoring the ribozyme sequence, the amount of sequence around the splice site used for folding can change results. I folded an arbitrary number of bases from the upstream and downstream sequences. As folding algorithms can give different results even when adding or removing single bases from the ends, determining the appropriate sequence context to fold is an additional challenge for accurate prediction.

As the splicing parameters are derived from RNA folding algorithms, having more accurate algorithms could certainly improve the accuracy of splicing prediction. In the second step of splicing, the IGS can base pair with both the 5′- and 3′-exons in a pseudoknot-like structure. Thus, programs that can handle pseudoknotted structures (such as kinefold) likely will be more accurate than programs that cannot. In addition, the transcribing polymerase (e.g., E. coli vs. T7) can affect activity, presumably due to effects on folding [79, 88]. A program like kinefold that can process co-transcriptional folding is likely more accurate but still unlikely to account for many secondary effects due to transcription [115].

Also, no folding algorithm considers how the ribosome will affect the RNA structure. Translation is required for splicing of the group I ribozymes from T4 phage [128, 131], with the ribosome unfolding incorrect pairings between the ribozyme and exon. For the cis-splicing GFP construct, there are also ribosomes at the splice site, and it is unknown how large a contribution these ribosomes may play in splicing. Using different folding algorithms can give qualitatively different results. Knowing which algorithm is the best to use is not an easy task. For example, for the 40 IGS variants, Pr(G₆:uₛ) as calculated by Vienna RNA versus the same probability calculated using kinefold only had an R²=0.57. Vienna RNA uses a standard partition folding algorithm based on energy minimization whereas kinefold considers the kinetic folding pathway. When substituting the Vienna RNA probabilities for the kinefold probabilities in step 2 of the model, the R² fit to the experimental data dropped from 0.74 to 0.56. I did not try many programs, but settled on kinefold because it empirically gave good results, handled pseudoknots, was reasonably fast, and had a programmable interface. Further research is needed to understand how the choice of algorithm affects the model.

### Conclusion

The data suggests that rational design of an efficient IGS is straightforward. For predicting splicing efficiencies, RNA folding algorithms can do reasonably well. A three step model with four free parameters could predict the splicing efficiency of 40 IGS variants with nearly 75% of the variation explained. The probability of a G₆:U pairing is the largest determinant of splicing efficiency and the ratio Pr(G₆:uₛ)=Pr(G₆:uₒₜₕₑᵣ) appears to be a reasonable heuristic for estimating splicing efficiency. More finegrained control of splicing can come from manipulating the interaction of the IGS with the 3′-exon.

### Example 2

### Ribozyme Engineering

If the splicing ribozyme is to become a core biological part usable for many applications, we should understand well the internal workings of the ribozyme. There is no better way to test and push our understanding than by engineering new ribozymes. Engineering new ribozymes also expands the family of usable splicing ribozymes. Although single ribozyme systems are useful, multi-ribozyme systems would be even more powerful. My efforts at constructing systems with two copies of the ribozyme near each other failed during cloning, always with one copy disappearing, likely due to recombination. By engineering new ribozymes with different sequences, recombination should be less of a problem.

Schultes and Bartel [130] showed that synthetic ribozymes could be designed to fall on a neutral path between two unrelated ribozymes. Each step on the path changed no more than 2 nt and preserved ribozyme activity. Along the path was one sequence that could adopt both ribozyme folds. Thus, ribozyme folding is highly flexible and relatively independent of the primary sequence. For splicing ribozymes, the secondary and tertiary structures are also more important than the primary sequence. To take advantage of this sequence flexibility, I designed new splicing ribozymes that have low primary sequence identity but high secondary and tertiary structural identity.

### Sequence Alignment Analysis

To understand the importance of each base in the ribozyme, I analyzed an alignment of 837 group IC1 ribozymes (the subgroup containing the Tetrahymena ribozyme) from the Group I Intron Sequence and Structure Database (GISSD) [175]. The alignment was processed to make structure information diagrams, similar to sequence and structure logos [55, 129], but instead of mapping information content on to a linear “logo,” bases are drawn as a secondary structure. The information content is not represented by the height of the base but rather by its color. The total information I(i) at position i in the alignment is calculated as

$$J ( i , b ) = f ( i , b ) \cdot {log}_{2} \frac{f ( i , b )}{0.25} \\ f ( i , b ) = \frac{n ( i , b )}{{\sum }_{b ∈ B} n ( i , b )} . \qquad ( Equation 7 )$$

where B={A,C, G,U}, n(i, −) is the number of sequences containing a gap at position i, n(i, b) is the number of sequences containing base b at position i, and

$$J ( i , b ) = f ( i , b ) \cdot {log}_{2} \frac{f ( i , b )}{0.25} \\ f ( i , b ) = \frac{n ( i , b )}{{\sum }_{b ∈ B} n ( i , b )} . \qquad ( Equation 8 )$$

The 0.25 indicates all four bases are expected to occur with equal frequency. Gaps in the alignment are handled using the method of Schneider and Stephens [129]. In calculating base frequencies, gaps are ignored, but the total information is reduced by the frequency of gaps. The color of a base is determined by f(i, b) ·I(i), which is between 0 and 2 bits. If J(i, b) is negative, the base is displayed upside down to indicate that it occurs less than expected [55]. In a sequence logo, bases at each position are stacked in order of increasing frequencies [129]. To reduce visual clutter, a structure information diagram only shows one base or gap at every position. However, multiple structure information diagrams can represent all the information in a sequence logo.

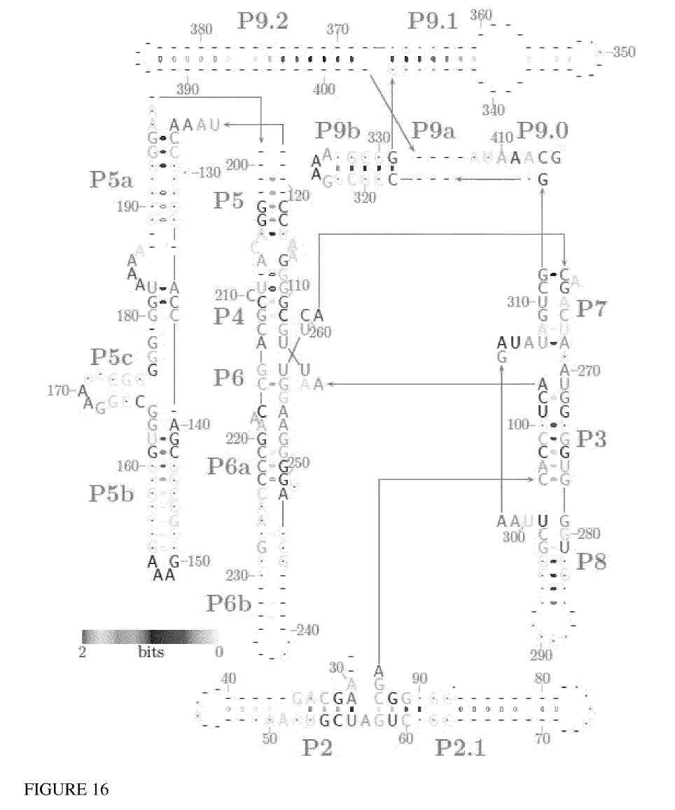

FIG. 16 shows the most frequent base (or gap) at each position in the alignment, mapped on to the secondary structure of the Tetrahymena ribozyme. If a gap occurs most frequently at a position, it is represented with a dash. A black dot is shown at positions where the base has less than 0.1 bits of information.

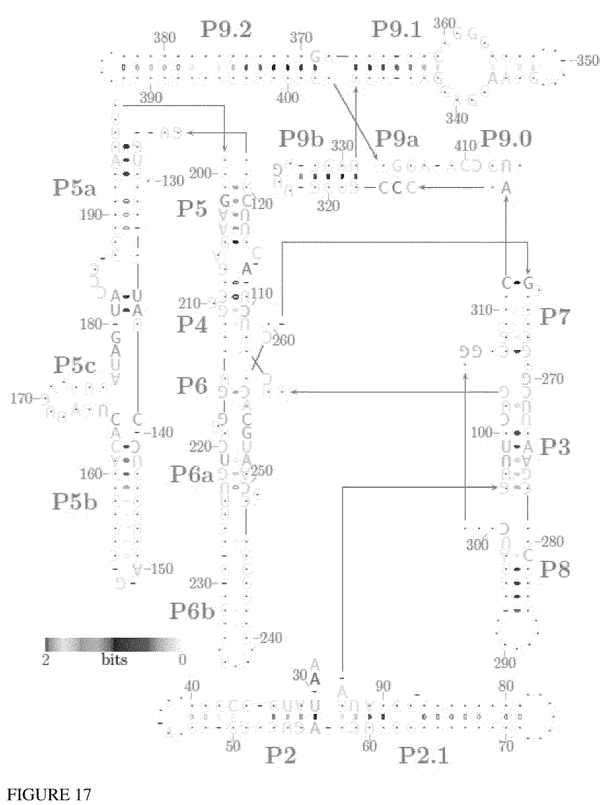

Similarly, FIG. 17 shows the second most frequent base (or gap) at each position. Although additional diagrams could be used to show even less frequent bases, no position contains more than about 0.1 bits of information in these other diagrams (data not shown). Just as in structure logos, base pairs can contain mutual information not found in the bases themselves [55]. For example, even if all four bases are found equally at two positions (zero positional information), the bases at the two positions could co-vary to always base pair (high mutual information). The additional information from a base pair is calculated using the log-likelihood ratio of the observed to the expected frequency of base pairing. Both frequencies are calculated after eliminating sequences with gaps. The observed frequency of a base pairing is calculated as the number of sequences with the base pairing divided by the number of sequences that contain a non-gap base at one or both of the positions in the base pair. The expected frequency of base pairing between positions i and j is equal to Σf(i, b)·f(j, c) for all combinations of bases b and c that can base pair (Watson-Crick plus G:U). As the base pairing for the Tetrahymena ribozyme in the alignment sometimes differed from the reference pairing (FIG. 3), I only used the base pairs common to both. The mutual information from base pairing is represented on the structure information diagram as the color of the base pairs and is drawn using the same scale as for the bases. Although the base pair information can be greater than 2 bits, it is capped at 2 bits. If the actual frequency of a base pair is less than the expected frequency, then the base pair is drawn in outline form, rather than completely filled.

### Mutagenesis

A standard splicing module with LacZα was the basis for mutagenesis. I swapped the bases in individual base pairs using site-directed mutagenesis. I also characterized some clones containing only single mutations. LacZα activity for each mutant was measured and normalized to the non-mutated construct.

### Synthetic Ribozymes

From the alignment and information known about each base in the ribozyme (Table 6), I generated a map of positions in the ribozyme where the identity of the base is likely unimportant (FIG. 18). “Harmless” bases are defined as positions having a total information content less than 0.05 and have no known tertiary interactions with other positions. “Likely mutable” bases have a maximum total information content of 0.25, may have some tertiary interactions, but can likely be changed with some care or additional experimentation. As an initial attempt at making synthetic ribozymes, I only made changes by swapping bases within a base pair. These swaps should maintain the secondary structure while changing the primary sequence.

FIG. 18 shows the design of the entire synthetic ribozyme, containing 152 base changes, which is 39% of the sequence. I constructed several synthetic ribozyme variants using standard techniques. All ribozyme variants replaced the native ribozyme in the cis-splicing GFP construct in the plasmid pSB1A3. To calculate splicing efficiency, the maximum GFP synthesis rate for the ribozyme variant was normalized to the native ribozyme.

### Alternative Ribozymes

One approach for expanding the number of ribozymes is to rely on the diversity that exists naturally. Although the alignment contained 837 ribozymes, many more sequences are found in this family. One disadvantage of relying on these other ribozymes is that most have never been characterized at all and may not function as a self-splicing ribozyme in a bacterial host. I tested two sequences in the alignment for their ability to function as a self-splicing ribozyme.

Cde.S943 from Coccomyces dentatus is the shortest intron found in the alignment with 217 nt. In the native context for Cde.S943, the G:U base pair occurs at position −5, rather than at −6 in Tetrahymena. The Cde.S943 ribozyme was cloned into an existing cis-splicing construct, directly replacing the Tetrahymena ribozyme and leaving the G:U base pair to form at position −6. Another variant had the 3′-most base of the IGS deleted so that the G:U base pair would be at position −5. However, neither IGS variant with Cde.S943 showed splicing activity. Some group I ribozymes require additional protein cofactors for folding [91, 106]. Cde.S943 lacks a P5abc domain, which is known to help stabilize the Tetrahymena ribozyme [83].

Hep.S943 from Hymenelia epulotica is a second intron that contains the P5abc domain and is roughly the same length as the Tetrahymena ribozyme (370 nt). The native IGS also forms roughly the same structure as found in Tetrahymena with the G₆:uₛ base pair at the same location. However, replacing the Tetrahymena ribozyme with Hep.S943 again showed no detectable splicing.

### Mutagenesis Characterization

As a start to systematically characterizing the Tetrahymena ribozyme, I swapped single base pairs and measured the relative splicing efficiency of the mutated ribozyme. The change in splicing efficiency indicates the importance of the bases beyond base pairing, such as additional stacking or tertiary interactions. I measured the efficiencies from nine base pair swaps, with most being in D₄₋₆. Table 6 lists the efficiencies of tested variants. As expected, switching the guanosine binding site 264:311 destroyed activity. All other base pair swaps maintained activity. The only base pair swap that was found to be truly neutral on splicing efficiency was 116:205. Some single base mutations were generated incidentally during site-directed mutagenesis and were also characterized. All single base mutations were worse than the compensatory double mutation, indicating the importance of base pairing and the secondary structure over the primary sequence.

### Synthetic Ribozymes

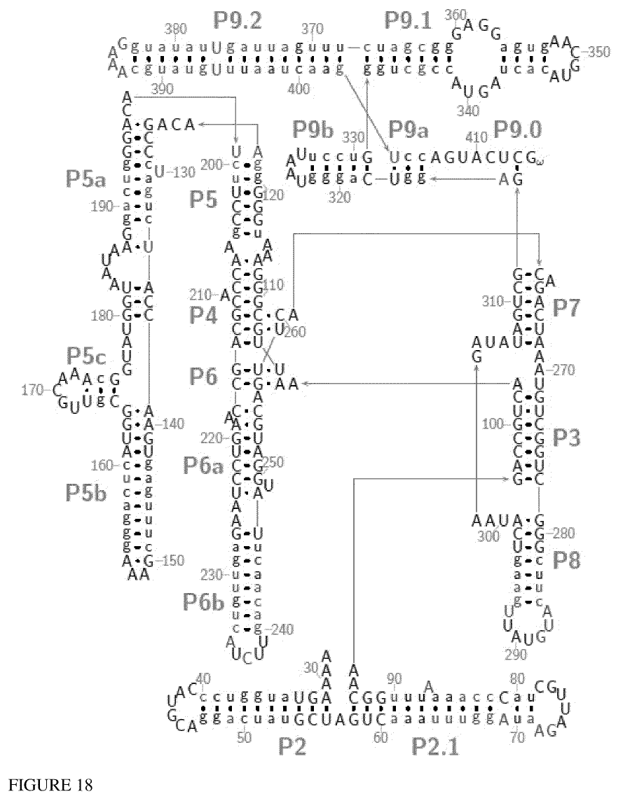

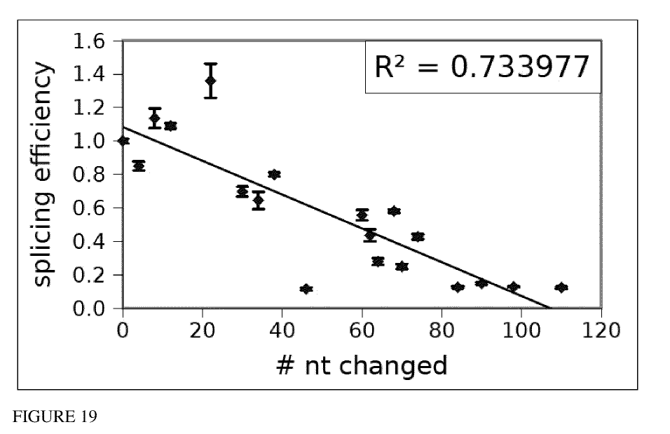

As the attempt to find alternative ribozymes that can self-splice was unsuccessful, another approach is to take the working Tetrahymena ribozyme and mutate it to create a new synthetic ribozyme. I designed a synthetic ribozyme (FIG. 18) by swapping bases intended to maintain the secondary structure. To test individual domains of the ribozymes, I constructed different synthetic ribozyme combinations. Table 4 shows the splicing efficiencies for the synthetic ribozyme variants. FIG. 19 shows that the splicing efficiency dropped relatively linearly as the number of nucleotides changed increased. However, two synthetic ribozymes did not follow the trend line. The ribozyme containing a synthetic P6b and P8 with 22 nt changed (SZ4) had significantly greater splicing efficiency than even the native ribozyme. On the other hand, the ribozyme with a synthetic D₄₋₆ (SZ8) showed a splicing efficiency significantly below the trend line. Large portions of the D₂ and D₉ peripheral domains can be mutated with only a modest decrease in splicing efficiency. Comparing SZ9-SZ11, it appears that the native base pairs 322:327 and 346:353 in D9 contribute to splicing efficiency and should not be changed. The ribozyme with the most number of changes, SZ18, contains mutations in all regions except P5 and P5abc and still had easily measurable splicing activity. The synthetic P6b and P8 helices (SZ2-SZ4) were the least disruptive and even had a slight beneficial effect for ribozyme splicing.

TABLE 4

| | | #nt | |
|---|---|---|---|
| Name | Regions changed | changed | Efficiency |
| SZ0 | none | 0 | 1.00 ± 0.01 |
| SZ1 | P5(116) | 4 | 0.85 ± 0.03 |
| SZ2 | P8 | 8 | 1.14 ± 0.06 |
| SZ3 | P6b(228) | 12 | 1.09 ± 0.02 |
| SZ4 | P6b, P8 | 22 | 1.36 ± 0.10 |
| SZ5 | D₂(70, 71) | 30 | 0.70 ± 0.03 |
| SZ6 | D₂ | 34 | 0.64 ± 0.05 |
| SZ7 | D₂(70, 71), P8 | 38 | 0.80 ± 0.01 |
| SZ8 | D₄₋₆ | 46 | 0.12 ± 0.01 |
| SZ9 | D₉(322, 346) | 60 | 0.56 ± 0.03 |
| SZ10 | D₉(346) | 62 | 0.44 ± 0.04 |
| SZ11 | D₉ | 64 | 0.28 ± 0.02 |
| SZ12 | P8, D₉(322, 346) | 68 | 0.58 ± 0.01 |
| SZ13 | P8, D₉(346) | 70 | 0.25 ± 0.02 |
| SZ14 | P2, D₉(322, 346) | 74 | 0.43 ± 0.02 |
| SZ15 | D₂(70, 71), D₉(322, 343-346) | 84 | 0.13 ± 0.01 |
| SZ16 | D₂(70, 71), D₉(322, 346) | 90 | 0.15 ± 0.01 |
| SZ17 | D₂(70, 71), P8, D₉(322, 346) | 98 | 0.13 ± 0.00 |
| SZ18 | D₂(70, 71), P6b(228), P8, D₉(322, 346) | 110 | 0.12 ± 0.01 |

The splicing efficiencies for synthetic ribozyme variants were normalized to the native ribozyme (SZ0).
Ribozyme changes are specified relative to FIG. 18 with native base pairs in parenthesis.
For example, D2(70, 71) indicates that all bases in the P2 and P2.1 helices use the synthetic ribozyme design in FIG. 18 except for the base pairs at positions 70 and 71 (70:80 and 71:79).
The number of bases different between each synthetic ribozyme and the native Tetrahymena sequence is shown, out of a total of 387 nt.

Mutations in the P5abc region appear to be the most detrimental. D₄₋₆ (SZ8) had low splicing efficiency but mutations in P5 (SZ1 and swap of 116:205) and P6b appeared to be benign. Thus, the P5abc region is the likely cause for the inefficiency of the SZ8 ribozyme. Four of the base pairs in P5abc were individually swapped and all showed reasonably efficient splicing. Either one of the untested base pairs in P5abc is responsible for significantly affecting splicing or the mutations in combination have a deleterious effect. The most likely detrimental mutation is the C166:G174 base swap. Both of these bases may form alternate base pairs during the folding process [164] and should not have been included in the synthetic ribozyme design.

### Alternative Ribozymes

One approach for obtaining a new splicing ribozyme is to use one of the many existing ribozymes in the family. Most of the ribozymes in the family were determined to be similar by sequence or structure alignment. Despite the large number of splicing ribozymes to determined by alignment, only several have ever been experimentally characterized. Other ribozymes that have been studied include the ribozymes from Azoarcus [68, 125], Pneumocystis [2, 13], Didymium iridis (DiGIR2) [49, 99], and Fuligo (Fse.L569 and Fse.L1898) [49].

To test the set of usable ribozymes, I selected two uncharacterized ribozymes from the alignment. At the primary sequence level, both Cde.S943 and Hep.S943 have a low number of bases common with the Tetrahymena ribozyme (98-99 nt). Cde.S943 is a short intron, lacking the P5abc domain, and failed to splice properly. Hep.S943 contains P5abc and has a similar secondary structure to the Tetrahymena ribozyme, but also failed to self-splice in vivo. These ribozymes may be inefficient due to the new sequence context or the new environment. For example, some group I ribozymes require additional protein cofactors and are incapable of self-splicing [52]. These results indicate that tweaking a working ribozyme may be better than using a random ribozyme from the family.

### Structure Information Diagram

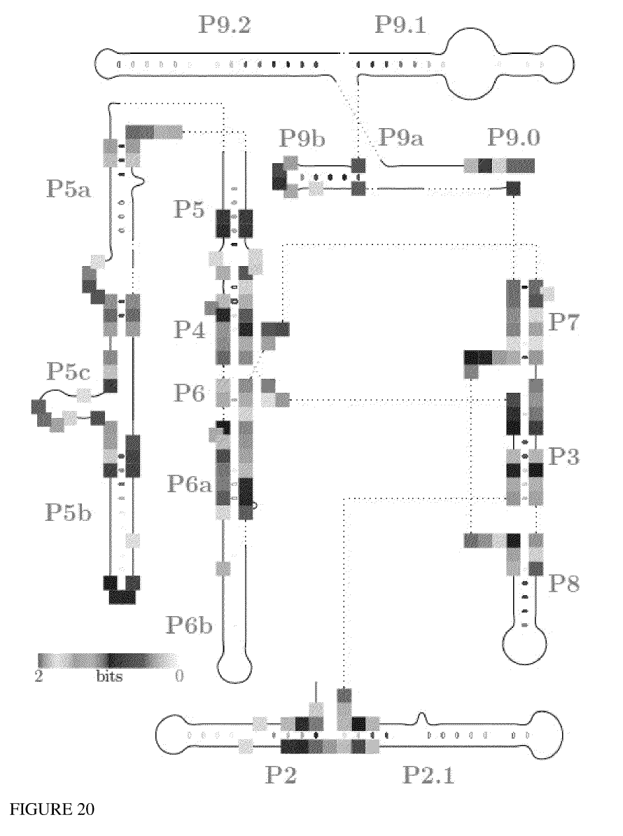

To help visualize sequence alignment information for large RNA structures, I developed the structure information diagram. The structure information diagram maps the information content found in sequence logos on to a secondary structure diagram to allow for a more natural visualization. FIG. 20 shows an abstracted version of the structure information diagram in FIG. 16. Colored boxes are shown instead of the bases. The catalytic core and the non-conserved peripheral regions can be easily visualized. Base pair information is also represented in the structure information diagram. 110:209 is a high information base pair that occurs much less than would be expected by chance.

Many sequences in the alignment are missing base 208 so that 110 pairs with 210 instead. Thus, the 110:209 base pair is an unusual base pair found in Tetrahymena but not in many other ribozymes. Some other high information base pairs are 262:312 and 116:205. 262:312 is about equally a G:C or C:G base pair. Thus, even though the individual bases do not have high information content, the base pair is conserved. Similarly, at the base pair 116:205, all of the pairings U:A, C:G, and G:U occur with high frequencies. These diagrams map the alignment on to the Tetrahymena structure, so only alignment positions for which the Tetrahymena ribozyme does not have a gap are shown. Table 5 shows all positions in the alignment with positive information content where the Tetrahymena ribozyme has a gap and the consensus base is not a gap. A position number like x:y indicates the yth base after base x in the Tetrahymena numbering. The limited number of such positions indicates that most conserved bases are present in the Tetrahymena ribozyme. The position with the largest information content, 207.1, indicates that many ribozymes contain an A between positions 207 and 208. When I inserted an A after position 207 in the Tetrahymena ribozyme, the ribozyme showed no splicing activity. Thus, there are limits to using sequence alignment to infer changes that can be made to the ribozyme.

TABLE 5

| Position | Information | Consensus |
|---|---|---|
| 126.1 | 0.5 | G |
| 168.1 | 0.3 | G |
| 168.2 | 0.2 | G |
| 172.19 | 0.2 | C |
| 172.20 | 0.2 | C |
| 178.1 | 0.2 | U |
| 178.2 | 0.7 | A |
| 207.1 | 1.2 | A |

The total information content and the consensus base are shown for positions in the alignment that are gaps in the Tetrahymena ribozyme.
Only positions with an information content of at least 0.1 bits and a non-gap consensus are shown.

### Experimental Mutagenesis

Although alignments can provide useful information about functionally important bases, ultimately, experiments are needed to test and verify our understanding of the ribozyme. As an initial effort at understanding how to manipulate the ribozyme, I generated a small set of base pair swaps and characterized the change in splicing efficiency. Base pairs that can be swapped without significantly altering splicing effciency would be good targets for future ribozyme engineering. Completing this work by measuring the effect from switching every base pair (around 125 total base pairs) is experimentally feasible and would help us better understand the core ribozyme.

### Synthetic Ribozymes

Very few bases of the primary sequence are strictly conserved in group I ribozymes. Even in the P7 catalytic core region, except for the guanosine binding site G264:C311, the primary sequence can be changed, whereas the secondary structure usually needs to be maintained [112, 113]. I designed synthetic ribozymes with new primary sequences while trying to maintain the secondary structure and splicing activity of the ribozyme. Using the available information about each base in the ribozyme, I generated lists of harmless and likely mutable bases. Around half of the bases can likely be changed (FIG. 18). To generate synthetic ribozymes, I switched base pairs and tested different groups of base swaps for splicing efficiency. All synthetic ribozymes had detectable splicing activity. A total of 152 different bases were changed and it is unexpected that so many bases can be changed without eliminating splicing activity. There is a difference in the free energies of stacked base pairs (e.g., GC:CG does not have the same energy as CG:GC). The fact that so many base pairs could be successfully switched is evidence of the ribozyme's robustness to folding conditions. However, an increased number of mutations generally reduced splicing efficiency.

Base changes can affect the folding process in subtle ways that are currently unpredictable. One way to work around possible folding problems is to use mutagenesis and selection on the designed ribozymes to bring the efficiency back up. Many more synthetic ribozyme variants could be generated. I did not attempt to mutate unpaired bases which is another source for generating many ribozyme variants. Some of the ribozyme domains can support more significant changes beyond base pair swaps. For example, ribozymes with inserted tags, coding sequences, or other payloads could be useful. P6b and P8 may be flexible enough to add significant amount of sequence. We can also likely add sequence to the D₂ and D₉ domains. The P3, P4, P5, P6, and P7 helices form the catalytic core and should be manipulated with caution. One region that is not well understood is P5abc, which is not conserved but mutations in this region can strongly affect splicing efficiency. P5abc is found in only a small number of group I ribozymes, but is essential for the Tetrahymena ribozyme [8, 89]. The D₄₋₆ domain containing P5abc folds quickly and helps with the correct folding and assembly of the slow-folding D_{3,7,8} domain [114, 170]. P5abc likely helps in the folding process by stabilizing the ribozyme through tertiary interactions. Adding P5abc in trans can rescue splicing from ribozymes missing this domain [155]. Destabilizing mutations in P5abc have been found to increase the rate of folding of D_{3,7,8 [}149]. If the native ribozyme normally enters a kinetic trap, then destabilizing P5abc can allow escaping the kinetic trap, leading to a faster overall folding rate. However, all P5abc mutants here showed less efficient splicing. Clarifying the contribution of P5abc towards splicing would enable engineering this region of the ribozyme.

Engineering a minimal ribozyme would provide a scaffold for new synthetic ribozymes and test the limits of our ribozyme knowledge. Nearly 75% of the ribozyme can be to deleted one section at a time without destroying activity in vitro [14]. Deleting the entire D₉ domain, except P9.0, produces a ribozyme more active than wild type. Deleting both P6b and D₉ is also more active than wild type. Deleting both D₂ and D₉ or both P5 and D₉ maintains activity whereas deleting both D₂ and P5 does not produce an active ribozyme. Using the available information, a minimal ribozyme should be relatively straightforward to design and test. Synthetic ribozymes can give us better ribozymes. Some of the ribozymes generated here were more efficient at splicing than the native ribozyme. Random selection would likely produce even better ribozymes. The ribozymes were not characterized beyond their ability to perform one cis-splicing reaction. There are other possible reactions catalyzed by the ribozyme. When Williams et al. [161] selected for new P5abc domains, they obtained ribozymes that could self-splice but were deficient in the 3′-hydrolysis reaction. As the 3′-hydrolysis reaction is an unproductive side reaction, ribozymes capable of splicing but unable to hydrolyze the 3′-exon would be an improvement. More work is needed to understand how to not only design equivalent ribozymes, but to design better ribozymes.

### Ribozyme Base Summary

Table 6 collects information about each base in the Tetrahymena ribozyme from the literature and characterization experiments described herein. Understanding the ribozyme core will facilitate its use as a standard and reusable component of engineered biological systems.

TABLE 6
This table collects information about each base in the Tetrahymena
ribozyme. Tertiary interactions or mutagenesis information is listed
for each base. The two alignment columns are the total information
content in bits (max 2.0) and the most common base (consensus)
found in the alignment. Notes in bold are observations found through
the course of this work. The splicing efficiencies of mutated constructs
are normalized to the native ribozyme and swap (x:y) indicates that the
bases at positions x and y in the ribozyme were swapped.

| Base | Alignment | | Notes | Refs |
|---|---|---|---|---|
| A28 | 0.3 | — | deletion increases specificity, | [70, 168] |
| | | | reduces fidelity, and weakens | |
| | | | substrate binding | |
| A29 | 0.6 | — | deletion increases specificity, | [70, 168] |
| | | | reduces fidelity, and weakens | |
| | | | substrate binding | |
| A30 | 0.3 | A | deletion increases specificity, | [70, 168] |
| | | | reduces fidelity, and weakens | |
| | | | substrate binding | |
| A31 | 0.7 | A | G26, U56, mutations kill activity | [97, 119] |
| G32 | 1.2 | G | | |
| U33 | 0.4 | C | | |
| U34 | 0.2 | A | | |
| A35 | 0.2 | G | | |
| U36 | 0.1 | — | | |
| C37 | 0.2 | — | | |
| A38 | 0.1 | — | | |
| G39 | 0.1 | — | tolerant to mutations | [119] |
| G40 | 0.0 | — | tolerant to mutations | [119, 120] |
| C41 | 0.0 | — | L5e, may be tolerant to | [97, 119, 120] |
| | | | mutations | |
| A42 | 0.1 | — | L5e, mutations kill activity | [97, 119, 120] |
| U43 | 0.0 | A | L5e, mutations kill activity | [97, 119, 120] |
| G44 | 0.1 | A | can mutate if compensate in L5e | [64, 97, 119, 120] |
| | | | (C170), insertions tolerated | |
| | | | before 44 | |
| C45 | 0.1 | — | can mutate if compensate in L5e | [97, 119, 120] |
| | | | (G169) | |
| A46 | 0.0 | — | L5e, mutations kill activity | [97, 119, 120] |
| C47 | 0.1 | — | tolerant to mutations | [119] |
| C48 | 0.1 | — | tolerant to mutations | [119] |
| U49 | 0.0 | U | | |
| G50 | 0.2 | A | | |
| G51 | 0.2 | A | | |
| U52 | 0.1 | A | | |
| A53 | 0.2 | U | | |
| G54 | 1.0 | G | | |
| C55 | 1.2 | C | | |
| U56 | 0.7 | U | A31, mutations kill activity | [119] |
| A57 | 1.5 | A | A95, G279, 5′-splice site | [42] |
| G58 | 1.7 | G | | |
| U59 | 0.8 | U | | |
| C60 | 0.4 | C | | |
| U61 | 0.0 | G | | |
| U62 | 0.2 | G | | |
| U63 | 0.1 | C | | |
| A64 | 0.0 | — | | |
| A65 | 0.0 | — | | |
| A66 | 0.0 | — | | |
| C67 | 0.0 | — | | |
| C68 | 0.0 | — | | |
| A69 | 0.0 | — | | |
| A70 | 0.1 | — | | |
| U71 | 0.0 | — | | |
| A72 | 0.0 | — | | |
| G73 | 0.0 | — | | |
| A74 | 0.1 | U | | |
| U75 | 0.1 | A | can mutate if compensate at | [74, 97] |
| | | | A352 | |
| U76 | 0.0 | A | can mutate if compensate at | [74, 97] |
| | | | A351 | |
| G77 | 0.0 | G | can mutate if compensate at | [74, 97] |
| | | | C350 | |
| C78 | 0.0 | — | can mutate if compensate at | [74, 97] |
| | | | G349 | |
| A79 | 0.0 | — | may interact with L9.1 | [74, 97] |
| U80 | 0.0 | — | may interact with L9.1 | [97] |
| C81 | 0.0 | — | may interact with L9.1 | [97] |
| G82 | 0.0 | — | | |
| G83 | 0.0 | — | | |
| U84 | 0.0 | — | | |
| U85 | 0.1 | — | | |
| U86 | 0.1 | — | | |
| A87 | 0.0 | C | | |
| A88 | 0.1 | G | | |
| A89 | 0.1 | G | | |
| A90 | 0.0 | C | | |
| G91 | 0.4 | G | | |
| G92 | 1.2 | G | | |
| C93 | 1.7 | C | C93A is active | |
| A94 | 0.4 | G | A94G = 0.83 | [36] |
| A95 | 2.0 | A | A57, 5′-splice site selection, all | [36, 42] |
| | | | mutations kill activity | |
| G96 | 1.4 | C | | |
| A97 | 1.5 | A | usually a purine, U300, A97G is | [97] |
| | | | active | |
| C98 | 1.2 | C | | |
| C99 | 0.5 | C | | |
| G100 | 0.1 | G | may affect folding (depends on | [114] |
| | | | IGS), | |
| | | | swap(100:274) = 0.86 ± 0.02 | |
| U101 | 1.2 | U | all mutations kill activity | [36] |
| C102 | 1.2 | C | A218, all mutations kill activity | [36, 62] |
| A103 | 0.9 | A | all mutations kill activity | [36] |
| A104 | 1.9 | A | C217, all mutations kill activity | [36, 62] |
| A105 | 1.8 | A | C216:G257, all mutations kill | [36, 62] |
| | | | activity | |
| U106 | 1.3 | U | U258, U106A = 1.10 | [36, 62] |
| U107 | 1.9 | U | all mutations kill activity | [36] |
| G108 | 1.9 | G | U259, all mutations kill activity | [36, 97, 105] |
| C109 | 1.1 | C | C260, A184, all mutations kill | [11, 36, 97, 105] |
| | | | activity | |
| G110 | 0.8 | G | A183 | [11] |
| G111 | 1.4 | G | | |
| G112 | 1.8 | G | | |
| A113 | 1.0 | G | A113G = 0.73 | [36] |
| A114 | 1.8 | A | all mutations kill activity | [36, 160] |
| A115 | 0.3 | A | all mutations retain activity, | [36] |
| | | | A115C = 0.70 | |
| G116 | 0.2 | U | G116U = 0.80 ± 0.02, | |
| | | | swap(116:205) = 1.03 ± 0.03 | |
| G117 | 1.0 | C | | |
| G118 | 1.0 | C | | |
| G119 | 0.4 | — | A325 | [62] |
| U120 | 0.1 | — | swap(120:201) = 0.77 ± 0.04 | |
| C121 | 0.0 | — | | |
| A122 | 0.0 | — | | |
| A123 | 0.4 | U | | |
| C124 | 0.4 | A | | |
| A125 | 0.6 | A | A324 | [62] |
| G126 | 0.6 | A | | |
| C127 | 0.5 | C | | |
| C128 | 0.4 | C | | |
| G129 | 0.1 | C | | |
| U130 | 0.0 | U | | |
| U131 | 0.1 | U | | |
| C132 | 0.0 | A | | |
| A133 | 0.1 | G | | |
| G134 | 0.1 | C | | |
| U135 | 0.0 | — | swap(135:187) = 0.88 ± 0.04, | |
| | | | U135A = 0.80 ± 0.02 | |
| A136 | 0.7 | A | | |
| C137 | 0.8 | C | A186 | [31] |
| C138 | 1.5 | C | | |
| A139 | 0.4 | — | rearrangement during folding | [164] |
| A140 | 0.7 | A | rearrangement during folding | [164] |
| G141 | 0.9 | G | | |
| U142 | 0.9 | C | | |
| C143 | 0.1 | G | swap(143:160) = 0.85 ± 0.06 | |
| U144 | 0.1 | G | | |
| C145 | 0.1 | C | swap(145:158) = 0.82 ± 0.06 | |
| A146 | 0.2 | C | | |
| G147 | 0.2 | G | | |
| G148 | 0.1 | G | | |
| G149 | 0.1 | G | | |
| G150 | 0.9 | G | A152, A153 | [31] |
| A151 | 1.1 | A | A248 | [31] |
| A152 | 1.1 | A | U224, G150, G250 | [31] |
| A153 | 1.1 | A | G150, C223:G250 | [31] |
| C154 | 0.2 | G | | |
| U155 | 0.1 | G | | |
| U156 | 0.1 | C | | |
| U157 | 0.1 | G | | |
| G158 | 0.1 | G | swap(145:158) = 0.82 ± 0.06 | |
| A159 | 0.0 | G | | |
| G160 | 0.1 | G | swap(143:160) = 0.85 ± 0.06 | |
| A161 | 0.9 | G | | |
| U162 | 0.4 | U | | |
| G163 | 0.5 | G | rearrangement during folding, | [164] |
| | | | Mg²⁺ | |
| G164 | 0.6 | G | pairs with U177 during folding, | [31, 164] |
| | | | Mg²⁺, A186, C137:G181 | |
| C165 | 0.8 | C | pairs with G176 during folding | [164] |
| C166 | 0.1 | C | pairs with G175 during folding | [164] |
| U167 | 0.3 | G | pairs with G174 during folding, | [149, 164] |
| | | | U167C speeds D_{3,7,8} folding, | |
| | | | swap(167:173) = 0.89 ± 0.03 | |
| U168 | 0.5 | G | pairs with A173 during folding, | [97, 164] |
| | | | L2 | |
| G169 | 0.6 | A | can mutate if compensate in L2 | [97] |
| | | | (C45) | |
| C170 | 0.7 | A | can mutate if compensate in L2 | [97] |
| | | | (G44) | |
| A171 | 0.1 | U | L2 | [97] |
| A172 | 0.1 | G | | |
| A173 | 0.1 | C | pairs with U168 during folding, | [164] |
| | | | swap(167:173) = 0.89 ± 0.03 | |
| G174 | 0.2 | G | pairs with U167 during folding | [164] |
| G175 | 0.1 | G | pairs with C166 during folding, | [149, 164] |
| | | | insertion of G before 175 speeds | |
| | | | D_{3,7,8} folding | |
| G176 | 0.9 | G | pairs with C165 during folding | [164] |
| U177 | 0.4 | G | pairs with G164 during folding | [164] |
| A178 | 0.7 | G | | |
| U179 | 0.4 | — | | |
| G180 | 1.6 | G | | |
| G181 | 0.8 | G | A186 | [31] |
| U182 | 0.6 | U | | |
| A183 | 1.3 | A | G110, all mutations kill activity, | [11, 31, 36, 149] |
| | | | A183U speeds D_{3,7,8} folding | |
| A184 | 0.8 | A | C109:G212 | [11, 31] |
| U185 | 0.6 | A | | |
| A186 | 1.8 | A | A186U disrupts D₄₋₆ structure | [31, 109, 149] |
| | | | and speeds D_{3,7,8} folding, | |
| | | | C137:G181 and G164 | |
| A187 | 0.1 | — | rearrangement during folding, | [164] |
| | | | swap(135:187) = 0.88 ± 0.04 | |
| G188 | 0.1 | — | pairs with U135 during folding, | [164] |
| | | | Mg²⁺ | |
| C189 | 0.1 | G | | |
| U190 | 0.0 | C | | |
| G191 | 0.1 | G | | |
| A192 | 0.0 | A | | |
| C193 | 0.1 | G | | |
| G194 | 0.4 | G | | |
| G195 | 0.5 | G | | |
| A196 | 0.2 | A | | |
| C197 | 0.1 | A | | |
| A198 | 0.1 | — | | |
| U199 | 0.0 | — | | |
| G200 | 0.0 | — | | |
| G201 | 0.0 | — | swap(120:201) = 0.77 ± 0.04 | |
| U202 | 0.5 | — | A325 | [62] |
| C203 | 1.0 | G | | |
| C204 | 1.1 | G | | |
| U205 | 0.2 | A | swap(116:205) = 1.03 ± 0.03 | |
| A206 | 0.3 | C | all mutations are okay | [36] |
| A207 | 0.4 | A | phosphate in active site, all | [36, 62, 160] |
| | | | mutations kill activity | |
| C208 | 0.0 | — | phosphate in active site, | [62] |
| | | | insertion of A before C208 | |
| | | | has zero activity | |
| C209 | 1.7 | U | | |
| A210 | 1.3 | C | deletion = 0.50 | [36] |
| C211 | 1.2 | C | A183 | [11] |
| G212 | 1.9 | G | A183, C260, all mutations kill | [11, 97, 105] |
| | | | activity, A184 | |
| C213 | 1.9 | G | U259, all mutations kill activity | [36, 97, 105] |
| A214 | 2.0 | A | all mutations kill activity | [36] |
| G215 | 1.7 | G | U258, U106, all mutations kill | [36, 62] |
| | | | activity | |
| C216 | 1.5 | C | A105, all mutations kill activity | [36, 62] |
| C217 | 1.2 | C | A104, C255 | [62] |
| A218 | 1.6 | A | C102:G272 | [62] |
| A219 | 1.7 | A | G254 | [62] |
| G220 | 0.9 | G | C255 | [97] |
| U221 | 0.7 | C | | |
| C222 | 0.6 | C | | |
| C223 | 0.7 | C | A153 | [31] |
| U224 | 0.3 | C | A152 | [31] |
| A225 | 0.2 | A | GAAA in L5b | [35, 97] |
| A226 | 0.2 | A | GAAA in L5b | [35, 97] |
| G227 | 0.0 | A | GAAA in L5b | [35, 97] |
| U228 | 0.4 | G | swap(228:246) = 0.82 ± 0.04 | |
| C229 | 0.1 | C | | |
| A230 | 0.0 | C | | |
| A231 | 0.0 | — | | |
| C232 | 0.1 | — | | |
| A233 | 0.0 | — | can be deleted | [120] |
| G234 | 0.0 | — | can be deleted | [120] |
| A235 | 0.0 | — | can be deleted | [120] |
| U236 | 0.1 | — | can be deleted, 50 nt insertion | [120] |
| | | | has no effect | |
| C237 | 0.0 | — | can be deleted | [120] |
| U238 | 0.1 | U | can be deleted | [120] |
| U239 | 0.1 | C | can be deleted | [120] |
| C240 | 0.0 | — | can be deleted | [120] |
| U241 | 0.0 | — | can be deleted | [120] |
| G242 | 0.0 | — | can be deleted | [120] |
| U243 | 0.0 | — | can be deleted | [120] |
| U244 | 0.1 | — | can be deleted | [120] |
| G245 | 0.2 | G | | |
| A246 | 0.1 | C | swap(228:246) = 0.82 ± 0.04 | |
| U247 | 0.1 | U | GAAA in L5b | [35] |
| A248 | 0.7 | A | A151, U224 | [31] |
| U249 | 0.1 | U | GAAA in L5b | [35] |
| G250 | 1.2 | G | A152, A153 | [31] |
| G251 | 1.1 | G | GAAA in L5b | [35] |
| A252 | 0.5 | G | | |
| U253 | 0.5 | G | C255 | [97] |
| G254 | 0.6 | A | A219 | [62] |
| C255 | 0.7 | A | C217 | [62] |
| A256 | 0.3 | G | G272, U273 | [62] |
| G257 | 1.5 | G | A105, all mutations kill activity | [36, 62] |
| U258 | 1.9 | U | U106, all mutations kill activity | [36, 62] |
| U259 | 1.6 | U | G108:C213, all mutations kill | [22, 36, 105] |
| | | | activity | |
| C260 | 2.0 | C | C109:G212, all mutations kill | [22, 36, 105, 114] |
| | | | activity, affects folding, deletion | |
| | | | has zero activity | |
| A261 | 2.0 | A | A265:U310, all mutations kill | [22, 36, 62, 160] |
| | | | activity | |
| C262 | 0.8 | C | G312, phosphate contacts Mg²⁺, | [36, 62] |
| | | | all mutations kill activity | |
| A263 | 1.8 | A | G312, A263U = 0.60 | [36, 62] |
| G264 | 2.0 | G | G binding, all mutations kill | [15, 22, 36, 62] |
| | | | activity, swap(264:311) changes | |
| | | | from G-binding to A-binding for | |
| | | | both steps, swap(264:311) = 0 | |
| A265 | 1.8 | A | G binding, A261, all mutations | [22, 36, 62, 113] |
| | | | kill activity | |
| C266 | 1.9 | C | A306, all mutations kill activity | [22, 36, 62] |
| U267 | 1.8 | U | all mutations kill activity | [22, 36] |
| A268 | 0.5 | A | all mutations kill activity | [22, 36] |
| A269 | 0.1 | A | A304, A269U = 1.10 | [36, 97] |
| A270 | 1.4 | A | all mutations kill activity | [22, 36] |
| U271 | 0.6 | U | U271A = 0.60 | [36, 62] |
| G272 | 1.3 | G | A218, A256, all mutations kill | [22, 36, 62] |
| | | | activity | |
| U273 | 1.0 | G | A256 | [62] |
| C274 | 0.1 | G | may affect folding (depends on | [114] |
| | | | IGS), | |
| | | | swap(100:274) = 0.86 ± 0.02, | |
| | | | C274G = 0.76 ± 0.03 | |
| G275 | 0.5 | G | | |
| G276 | 1.2 | G | | |
| U277 | 1.4 | U | | |
| C278 | 1.4 | G | | |
| G279 | 1.9 | G | A57, A95, all mutations lower | [36, 42] |
| | | | activity | |
| G280 | 1.7 | G | | |
| G281 | 0.8 | U | G281A is active | |
| G282 | 0.1 | U | | |
| A283 | 0.2 | G | | |
| A284 | 0.0 | G | | |
| G285 | 0.0 | G | | |
| A286 | 0.0 | — | | |
| U287 | 0.0 | — | | |
| G288 | 0.0 | G | | |
| U289 | 0.1 | C | | |
| A290 | 0.1 | A | | |
| U291 | 0.1 | A | | |
| U292 | 0.0 | — | | |
| C293 | 0.0 | C | | |
| U294 | 0.0 | G | | |
| U295 | 0.1 | C | | |
| C296 | 0.1 | G | | |
| U297 | 1.4 | G | U297C is active | |
| C298 | 1.5 | C | | |
| A299 | 1.2 | U | A299U = 0.65 | [36] |
| U300 | 1.8 | U | A97, all mutations kill activity | [36, 97] |
| A301 | 1.9 | A | all mutations kill activity | [22, 36, 160] |
| A302 | 2.0 | A | all mutations kill activity | [22, 36] |
| G303 | 1.9 | G | all mutations kill activity | [22, 36, 160] |
| A304 | 1.2 | A | A269, all mutations kill activity | [22, 36, 97] |
| U305 | 1.2 | U | phosphate contacts Mg²⁺, all | [22, 36, 62, 159] |
| | | | mutations kill activity | |
| A306 | 1.9 | A | C266:G309, phosphate contacts | [22, 36, 62] |
| | | | Mg²⁺, all mutations kill activity | |
| U307 | 0.4 | U | all mutations kill activity | [22, 36] |
| A308 | 1.8 | A | important for second splicing | [22, 36, 113] |
| | | | step, all mutations kill activity | |
| G309 | 1.9 | G | A306, all mutations kill activity | [22, 36, 62] |
| U310 | 1.9 | U | A261, all mutations kill activity | [22, 36, 62, 160] |
| C311 | 1.9 | C | G binding, all mutations kill | [15, 22, 36, 62] |
| | | | activity, affects folding, | |
| | | | swap(264:311) changes from | |
| | | | G-binding to A-binding for both | |
| | | | steps, swap(264:311) = 0 | |
| G312 | 0.8 | G | C262, A263, all mutations kill | [36, 62] |
| | | | activity | |
| G313 | 1.0 | G | C413 | [62] |
| A314 | 0.0 | U | | |
| C315 | 0.3 | — | | |
| C316 | 0.6 | — | | |
| U317 | 0.3 | — | | |
| C318 | 0.7 | C | | |
| U319 | 0.2 | C | | |
| C320 | 0.2 | G | | |
| C321 | 0.3 | C | | |
| U322 | 0.1 | G | | |
| U323 | 1.5 | G | | |
| A324 | 1.2 | A | A125 | [62] |
| A325 | 0.9 | A | G119:U202 | [62] |
| U326 | 1.5 | A | | |
| G327 | 0.1 | G | | |
| G328 | 0.2 | G | | |
| G329 | 0.2 | C | | |
| A330 | 0.2 | G | | |
| G331 | 0.8 | G | | |
| C332 | 0.1 | U | | |
| U333 | 0.1 | C | | |
| A334 | 0.1 | C | | |
| G335 | 0.0 | A | | |
| C336 | 0.0 | — | | |
| G337 | 0.1 | — | | |
| G338 | 0.2 | — | | |
| A339 | 0.1 | — | | |
| U340 | 0.1 | — | | |
| G341 | 0.2 | — | | |
| A342 | 0.1 | — | | |
| A343 | 0.2 | — | | |
| G344 | 0.1 | — | | |
| U345 | 0.0 | — | | |
| G346 | 0.1 | — | may interact with L2.1 | [74, 97] |
| A347 | 0.0 | — | may interact with L2.1 | [74, 97] |
| U348 | 0.0 | — | may interact with L2.1 | [74, 97] |
| G349 | 0.0 | — | can mutate if compensate at C78 | [74, 97] |
| C350 | 0.1 | A | can mutate if compensate at G77 | [74, 97] |
| A351 | 0.1 | A | can mutate if compensate at U76 | [74, 97] |
| A352 | 0.1 | — | can mutate if compensate at U75 | [74, 97] |
| C353 | 0.1 | — | | |
| A354 | 0.0 | — | | |
| C355 | 0.1 | — | | |
| U356 | 0.1 | — | | |
| G357 | 0.2 | — | | |
| G358 | 0.2 | — | | |
| A359 | 0.2 | — | | |
| G360 | 0.1 | — | | |
| C361 | 0.2 | — | | |
| C362 | 0.1 | — | | |
| G363 | 0.0 | — | | |
| C364 | 0.0 | U | | |
| U365 | 0.0 | G | | |
| G366 | 0.1 | G | | |
| G367 | 0.0 | G | | |
| G368 | 0.1 | — | | |
| A369 | 0.3 | — | | |
| A370 | 0.0 | — | | |
| C371 | 0.1 | — | | |
| U372 | 0.0 | — | | |
| A373 | 0.0 | — | | |
| A374 | 0.0 | — | | |
| U375 | 0.0 | — | | |
| U376 | 0.0 | — | | |
| U377 | 0.0 | — | | |
| G378 | 0.0 | — | | |
| U379 | 0.0 | — | | |
| A380 | 0.0 | — | | |
| U381 | 0.0 | — | | |
| G382 | 0.0 | — | | |
| C383 | 0.0 | — | | |
| G384 | 0.0 | — | | |
| A385 | 0.0 | — | | |
| A386 | 0.0 | — | | |
| A387 | 0.0 | — | | |
| G388 | 0.0 | — | | |
| U389 | 0.0 | — | | |
| A390 | 0.0 | — | | |
| U391 | 0.0 | — | | |
| A392 | 0.0 | — | | |
| U393 | 0.0 | — | | |
| U394 | 0.0 | — | | |
| G395 | 0.0 | — | | |
| A396 | 0.1 | — | | |
| U397 | 0.0 | — | | |
| U398 | 0.1 | — | | |
| A399 | 0.0 | — | | |
| G400 | 0.1 | — | | |
| U401 | 0.0 | — | | |
| U402 | 0.1 | — | | |
| U403 | 0.0 | — | | |
| U404 | 0.0 | — | | |
| G405 | 0.2 | — | | |
| G406 | 0.1 | — | | |
| A407 | 0.1 | — | | |
| G408 | 0.1 | A | | |
| U409 | 0.2 | U | 15 nt insertion tolerated | |
| | | | before 409 | |
| A410 | 0.4 | A | | |
| C411 | 0.9 | A | | |
| U412 | 0.3 | A | | |
| C413 | 0.7 | C | G313, A263, C262:G312 | [62] |
| G414 | 2.0 | G | G_{w}, 3′-splice site selection | [62, 160] |

### Example 3

### Trans-Splicing

Trans-splicing ribozymes allow rewriting RNA. In trans-splicing, there are two separate RNAs: the “target” and the “ribozyme.” For simplicity, I use ribozyme to refer to the RNA containing the ribozyme, including both the ribozyme and anything attached to it. I assume the target sequence is fixed and that the goal is to modify the target RNA by designing a ribozyme construct.

### Anti-IGS Region

During initial attempts at constructing trans-splicing ribozymes using the design of Kohler et al. [89], I saw apparent toxicity of certain constructs. Ribozymes without its intended target also present in the cell were particularly likely to be difficult to clone. In addition, the toxicity disappeared when the G₆ in the IGS was changed to another base or when an inactive ribozyme mutant was used. I hypothesized that the ribozyme could be erroneously splicing on to random cellular RNA, leading to cellular toxicity. To avoid non-specific splicing, I added a cis-anti-IGS to trans-splicing ribozymes. The anti-IGS pairs with the IGS and sequesters it with a G₆:C pairing, which is less likely to splice than a G₆:U. This to pairing prevents premature splicing but should be opened up after binding of the target RNA with the antisense sequence. The anti-IGS is represented as in figures.

### Trans-Knockdown

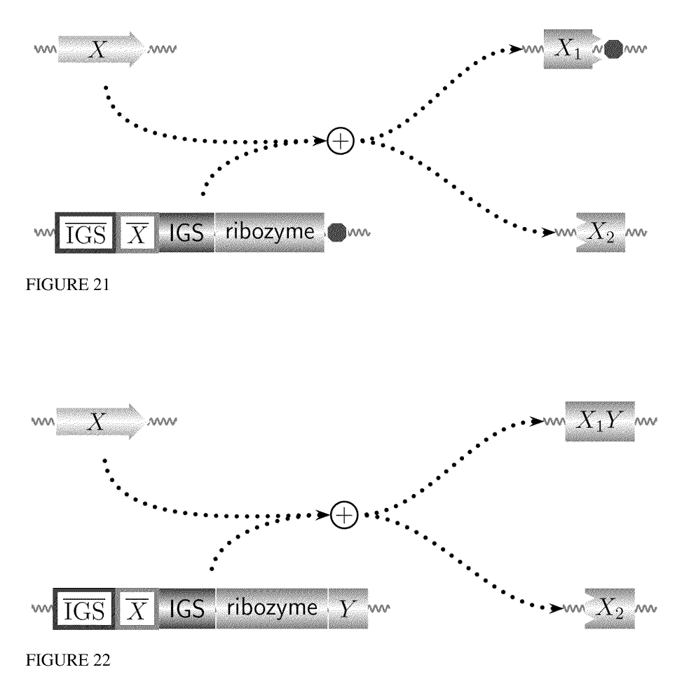

For trans-splicing, the ribozyme must first find the target RNA. After the target RNA is brought near the ribozyme, the remaining steps are identical to cis-splicing. I used trans-knockdown to test whether a ribozyme can find a target RNA. FIG. 21 shows the trans-knockdown of a coding sequence. The ribozyme splits the target RNA, splicing in a premature stop codon. Knockdown could occur in one of several ways. The antisense region could bind the target leading to antisense-mediated inhibition, independent of the ribozyme. Knockdown could also occur if the ribozyme binds the target and only performs the first step of splicing, cleaving the target RNA. Finally, knockdown would also occur if the stop codon is trans-spliced on to the target. In all of these cases, knockdown implies accessibility of the target RNA to the ribozyme. Thus, trans-knockdown is a necessary prerequisite for trans-splicing. I use anti(X) to refer to a trans-knockdown ribozyme that targets X.

### Trans-Splicing

The trans-knockdown ribozyme can be easily extended to trans-splicing by replacing the stop codon with another sequence Y (FIG. 22). This circuit implements a logical AND gate as the spliced outputs depend on both the target and ribozyme RNAs ([X₁Y]=[X]̂[ribozyme]). This trans-splicing operation can also be viewed as an RNA converter (X→X₁Y).

### Methods

All ribozymes were on the high-copy plasmid pSB1A3 and all trans-RNA targets were on the low-copy plasmid pSB4C5. All RNAs were transcribed using the constitutive promoter BBa_R0040 and all reporter genes used the RBS BBa_B0034. I measured GFP, mCherry, and LacZα expression to characterize splicing. A reference plasmid containing only BBa_R0040 was used to normalize the activity of all ribozyme constructs.

### Trans-Knockdown Constructs

The design for trans-knockdown ribozymes contained several components in addition to the ribozyme: an anti-IGS, an antisense region, a spacer, the IGS, and stop codons (FIG. 21). The 3′-exon for all knockdown ribozymes was CUAACUAACUAA, which contains a to stop codon in every reading frame. The IGS had a 9 by P1 and 4 by P10 region. Between the IGS and the antisense region, a spacer was inserted to provide structural flexibility so that the antisense pairing does not interfere with catalysis [89]. To knockdown gfp, I targeted ribozymes at the fluorophore of GFP (Tyr66) [152]. I inserted 5 As as a spacer between the IGS and the antisense region. Unless otherwise stated, the anti-IGS formed 13 by starting from position −4 of the IGS and included 3 nt of the spacer. The antisense sequence was complementary to the target gfp sequence starting from 5 bases after the 3′-end of the P1 pairing.

To knockdown lacZα, the ribozyme targeted the Val10 codon of lacZα. The ribozyme had a spacer of two As. The anti-IGS formed 14 by starting from −3 of the IGS and included one common base with the antisense sequence. The 81 nt antisense sequence matched the target lacZα starting from 6 bases after the 3′-end of the P1 pairing.

### Trans-Splicing Constructs

I designed ribozymes to trans-splice lacZα on to either gfp or mcherry RNA transcripts. For targeting gfp, I replaced the stop codons of a gfp knockdown ribozyme (81 nt antisense) with lacZα. For targeting mcherry, I constructed a trans-splicing ribozyme containing a 97 nt antisense sequence, a 5 nt spacer, and a 13 nt anti-IGS to base pair with the entire IGS. I removed the first three codons, including two possible start codons, from the lacZα coding sequence to eliminate possible background expression of non-spliced lacZα. Splicing at the expected site would form a fusion protein consisting of part of GFP or mCherry, followed by a SNYGGGGS peptide linker, and then an in-frame LacZα. The linker sequence began with CGAACUAU, which allows using the same IGS used in the trans-knockdown ribozymes, due to an identical P10 region (bolded). To test whether any splicing was occurring in another reading frame, I made alternate linkers by inserting one or two bases before the GGGGS codons.

### Trans-Knockdown

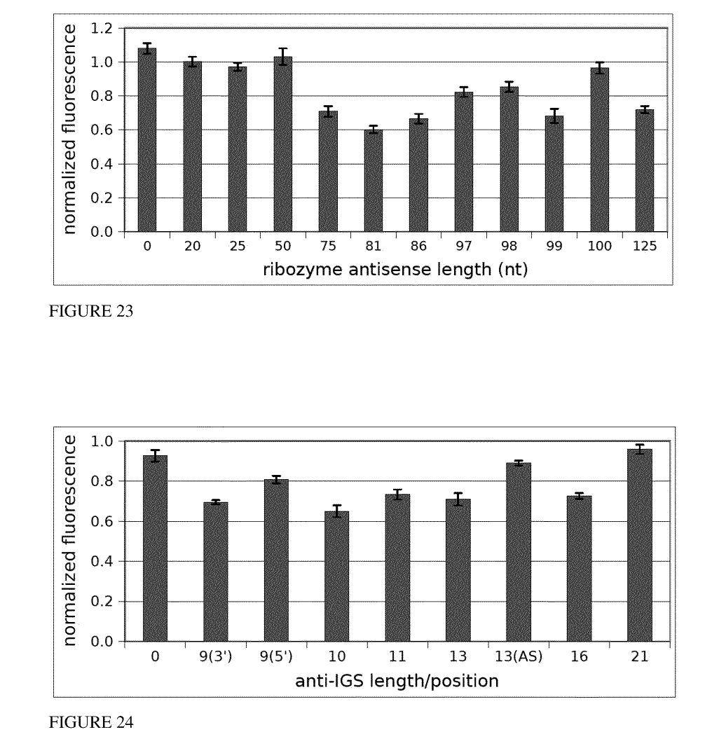

Using the design of FIG. 21, I targeted gfp or lacZα for trans-knockdown. gfp knockdown To determine the design rules for efficient trans-knockdown, I tested many variants of a gfp knockdown ribozyme. First, I varied the length of the antisense region from 0-125 nt. FIG. 23 shows the GFP fluorescence relative to a reference strain containing the target GFP only. All antisense lengths less than 50 nt showed little knockdown. Longer antisense sequences, however, did show noticeable knockdown, up to 40% knockdown with an antisense length of 81 nt. Using an antisense length of 75 nt, I varied the length and position of the anti-IGS. FIG. 24 shows the GFP knockdown from anti-IGS variants. Both no anti-IGS (0 nt) and a long anti-IGS (21 nt) had little knockdown. Anti-IGS lengths between 9-16 nt showed mostly comparable knockdown. The 13(AS) construct had the anti-IGS region shifted to be complementary to the 3′-end of the antisense region instead of the IGS. 13(AS) showed less knockdown than an anti-IGS of the same length targeted at the IGS, indicating that the knockdown was not likely only due to a 5′-hairpin stabilizing the ribozyme RNA. The 9(5′) construct and 9(3′) construct compared the effect of pairing with the nine bases on the 3′-end of the IGS versus the nine bases on the 5′-end of the IGS. Targeting the 3′-end showed greater knockdown, indicating that the effect is likely more complex than just the strength of the anti-IGS:IGS pairing. After determining that both the antisense and anti-IGS regions were necessary for trans-knockdown, I generated additional variants to test the mechanism behind the knockdown effect. Table 7 shows the activity from several additional trans-knockdown ribozymes based on the anti(gfp)-1 ribozyme with an 81 nt antisense and 13 nt anti-IGS region. Again, removing the anti-IGS (anti(gfp)-2) eliminated knockdown.

Several variants (anti(gfp)-3-8) tested if the IGS region affects knockdown. Mutating the G in the IGS that forms the critical G₆:uₛ to an A (anti(gfp)-3) had a minor decrease in knockdown. Extending the P10 to 8 by (anti(gfp)-4) or adding several extra Gs to the IGS (anti(gfp)-5) also showed a small decrease in knockdown. However, randomizing the entire IGS (anti(gfp)-6) eliminated knockdown. In anti(gfp)-6, the anti-IGS cannot pair with the IGS. To test if the effect from IGS randomization was due to a non-matching anti-IGS, in anti(gfp)-7, the anti-IGS was changed to match the randomized IGS and knockdown was restored. The ribozyme in anti(gfp)-1 was targeted at an alternative GFP variant (BBa_E0040) containing an identical antisense pairing region but with several mutations expected in the IGS pairing (anti(gfp)-8). Although the G₆:uₛ pairing could still conceivably form, the P1 helix was expected to be only 5 bp. The knockdown effect for anti(gfp)-8 was nearly identical to anti(gfp)-1. These results all suggest that the anti-IGS:IGS pairing is more important than the identity of the IGS.

I made ribozyme mutants to test if knockdown was due to splicing. Although a G264A ribozyme mutant showed less knockdown (anti(gfp)-9) than an active ribozyme, swapping the 264:311 guanosine binding site (anti(gfp)-10) did not reduce knockdown. A larger deletion in the ribozyme (anti(gfp)-11) reduced the knockdown whereas deleting the entire ribozyme (anti(gfp)-12) had a greater knockdown than when the ribozyme was present. Deleting both the ribozyme and portions of the IGS (anti(gfp)-13-15) showed varying to knockdown, with the greatest knockdown seen when 6 nt from the 3′ end of the IGS was removed. These experiments indicate that the knockdown was not due to the ribozyme or splicing.

TABLE 7
A target GFP was co-transformed with trans-knockdown ribozyme
variants. All ribozymes contain an 81 nt antisense region and a
13 nt anti-IGS, except anti(gfp)-2 which was missing the anti-IGS.
The anti(gfp)-1 construct was the base trans-knockdown ribozyme
from which the other variants were generated. A lower fluorescence
indicates a greater knockdown effect.

| Construct | Description | Fluorescence |
|---|---|---|
| anti(gfp)-1 | base ribozyme | 0.60 ± 0.02 |
| anti(gfp)-2 | Δ(anti-IGS) | 0.93 ± 0.03 |
| anti(gfp)-3 | IGS₆^{A} | 0.69 ± 0.03 |
| anti(gfp)-4 | 8 bp P10 | 0.65 ± 0.02 |
| anti(gfp)-5 | IGS₄₋₈^{G} | 0.70 ± 0.03 |
| anti(gfp)-6 | random IGS | 0.95 ± 0.02 |
| anti(gfp)-7 | random IGS + matched anti-IGS | 0.69 ± 0.01 |
| anti(gfp)-8 | target GFP with weak P1 | 0.57 ± 0.05 |
| anti(gfp)-9 | G264A | 0.70 ± 0.03 |
| anti(gfp)-10 | G264C, C311G | 0.58 ± 0.01 |
| anti(gfp)-11 | Δ(228-331) | 0.88 ± 0.04 |
| anti(gfp)-12 | Δ(ribozyme) | 0.46 ± 0.01 |
| anti(gfp)-13 | Δ(ribozyme, IGS₁₋₃) | 0.59 ± 0.01 |
| anti(gfp)-14 | Δ(ribozyme, IGS₁₋₆) | 0.40 ± 0.01 |
| anti(gfp)-15 | Δ(ribozyme, IGS) | 0.57 ± 0.01 |

### LacZ Knockdown

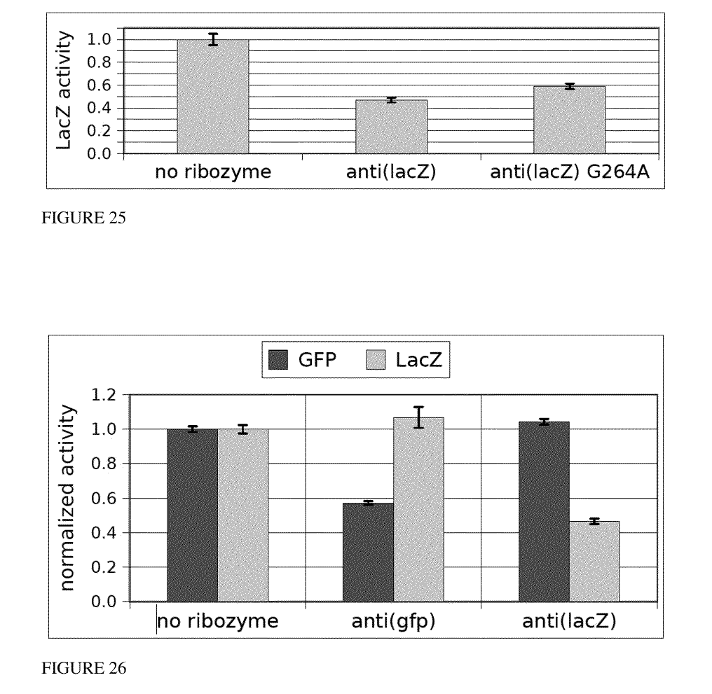

To test if trans-knockdown can work with a target other than gfp, I generated an anti(lacZ) trans-knockdown ribozyme with an 81 nt antisense region. FIG. 25 shows that this construct was capable of knocking down LacZ activity by more than half. Again, the G264A point mutation in the ribozyme showed reduced but still significant knockdown.

### Target Specificity

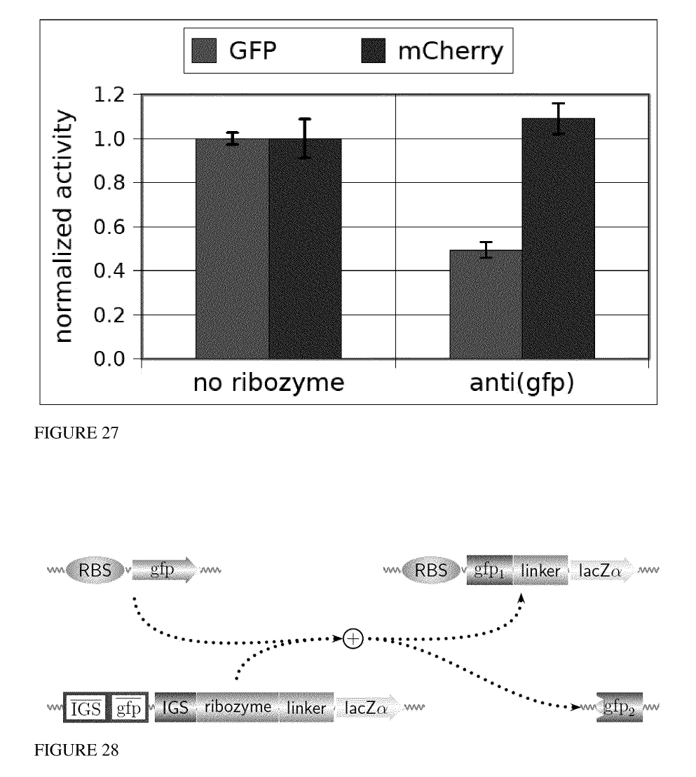

To test the specificity of the knockdown ribozymes, I combined the gfp and lacZα targets on to the same plasmid, each expressed on a separate RNA. This dual reporter plasmid was co-transformed with ribozymes targeting one of the two reporters. The ribozymes contained an 81 nt antisense region to their intended targets. The off-target reporter measures the specificity of the ribozyme targeting and also controls for effects like increased cellular to load. For example, if a ribozyme erroneously splices on to a critical cellular RNA, the expression of all RNAs in the cell could decrease due to the general unhealthiness of the cell. The ribozymes showed specificity in knocking down their intended target while not affecting the expression of the off-target reporter (FIG. 26). In another specificity test, I transformed an anti(gfp) ribozyme with a target plasmid expressing both gfp and mcherry. FIG. 27 again shows the specificity of the ribozyme in knocking down its intended target.

### Trans-Splicing

To definitively show trans-splicing, I designed ribozymes to replace a target RNA with a reporter gene. FIG. 28 shows a gfp→lacZα converter ribozyme, which trans-splices lacZα on to a gfp target. The ribozyme creates a protein fusion using part of GFP and LacZα. LacZ activity requires both the ribozyme and the target. Similarly, I designed mcherry→lacZα ribozymes to replace mcherry with lacZα. A linker sequence was inserted during splicing. To confirm that splicing occurs at, and only at, the expected location, I tested different linkers to put LacZα into the three possible reading frames. Reading frame 1 was the reading frame needed for an in-frame fusion of GFP and LacZα if the ribozyme splices at the expected location. Reading frames 2 and 3 were the other two reading frames

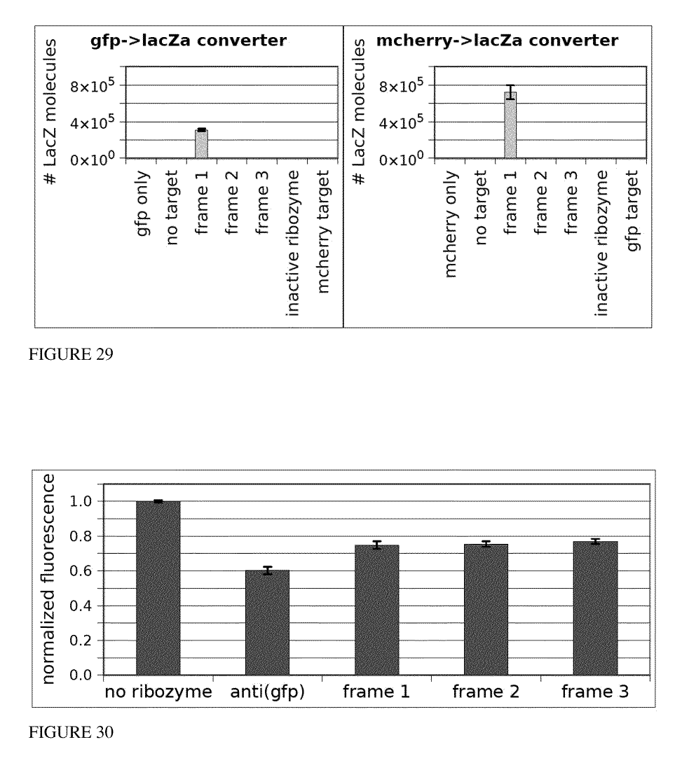

FIG. 29 shows LacZ activity for gfp→lacZα and mcherry→lacZα trans-splicing ribozyme variants. Most constructs had undetectable (zero) activity and only two constructs showed non-zero LacZ activity. LacZ activity was only detectable in cells with both the matched target and a trans-splicing ribozyme containing a linker for reading frame 1. Using a G264A ribozyme mutant also eliminated activity. These results indicate that the ribozymes were likely trans-splicing at the expected site. The LacZ activity for these ribozymes was extremely low and near the detection limit of the assay. For the gfp→lacZα converter, only about 30% of the “frame 1” colonies had non-zero values. Using a LacZ reference standard, the detection limit of the assay is around 2·10⁵, which is near the activity for this gfp→lacZα construct. To avoid introducing large non-real variation into the data, the results for this construct only averaged the non-zero values. Thus, the real activity is lower than shown. All other constructs had consistently zero or non-zero activity for all colonies. Not only should lacZα be produced by the trans-splicing reaction, the activity of the target reporter should be reduced. To test knockdown, I measured the GFP fluorescence for the three gfp→lacZα ribozymes (FIG. 30). These ribozymes were identical to the previous anti(gfp)−1 knockdown ribozyme, except that the stop codons in the 3′-exon had been replaced with a linker and lacZα. Although the knockdown effect was not as large as with the trans-knockdown ribozyme, all three trans-splicing ribozymes showed similar levels of knockdown. Thus, the gfp→lacZα ribozyme in reading frame 1 reduced the expression of GFP at the same time that it increased LacZ activity. The ribozymes with shifted reading frames only showed GFP knockdown without production of active LacZ. I made additional mcherry→lacZα ribozyme variants to test the importance of the anti-IGS region. The previous anti-IGS designs all formed Watson-Crick base pairs with the entire IGS. The critical G₆ in the IGS made an expected G₆:C base pair in a relatively strong 13 by helix. This anti-IGS:IGS pairing could inhibit splicing if it were not unwound upon binding of the target RNA. FIG. 31 shows that mutating the C in the G₆:C base pair to anything else increased the splicing efficiency. The G₆:A and G₆:G base pairs showed identical splicing efficiencies of about three times the G₆:C base pair. The G₆:U base pair had an unexpectedly high intermediate splicing efficiency. This G₆:U base pair should allow the ribozyme to cis-splice at the U, making trans-splicing impossible. Perhaps the strong anti-IGS pairing inhibited the second step of cis-splicing by preventing the P10 helix from forming. Eliminating the anti-IGS entirely showed the highest LacZ activity. Thus, the anti-IGS is detrimental for trans-splicing efficiency. The IGS can affect trans-splicing efficiency. As a step towards understanding how to design an IGS for improved efficiency, I increased the P10 pairing to 6 by from 4 by and found only a slight beneficial effect (<10% increase). Previous results also showed a 4 by P10 was sufficient for full activity [89]. Thus, the IGS is likely not limiting for trans-splicing efficiency.

### Trans-Knockdown Mechanism

The trans-knockdown ribozymes appeared to function but not due to a splicing mechanism. The specificity results indicate that the knockdown was not due to non-specific effects, such as growth defects. I discuss some possible mechanisms behind the observed trans-knockdown effect.

Antisense Effect

The simplest explanation for the knockdown is an antisense mechanism [141]. The antisense sequence could possible cause degradation or inhibit translation of the target. However, if the knockdown mechanism is primarily one of antisense, it is unclear how changes outside of the antisense region affect knockdown. For example, changing the anti-IGS or IGS decreased trans-knockdown activity. In addition, changing the 3′-exon, which is far from the antisense sequence, affected trans-knockdown efficiency (FIG. 30). The to difference seen between a 99 nt and 100 nt antisense region was large (FIG. 23). It is unintuitive that increasing the antisense sequence by a single nucleotide could lead to less knockdown. A previously published results also showed a similar non-monotonic effect, with an optimal antisense length of 293 nt and decreased efficiencies for both shorter and longer lengths [8]. The ribozyme with a 99 nt antisense region can form an extra 2 by for the anti-IGS:IGS interaction. Thus, perhaps the effect of changing the antisense length was due to changing the strength of the anti-IGS:IGS pairing. The length and stability of stem regions can affect antisense RNA stability [141].

Different antisense lengths and surrounding sequences may affect the folding of the antisense region and its accessibility for pairing with the target. For example, the G264A mutation in the ribozyme showed reduced knockdown for both the anti(gfp) and anti(lacZ) knockdown constructs. Perhaps this G264A point mutation changed the folding of the ribozyme, which then affected the folding of the antisense region and how well the antisense could bind to the target. In support of this hypothesis, a ribozyme with a compensatory double mutation at 264:311 instead of the G264A single mutation, showed no change in knockdown. The single mutation is likely to affect the secondary structure more than a double mutation that maintains base pairing. However, in vitro experiments showed no changes in the global folding of the G264A ribozyme [96].

Thus, further work is needed to understand the interactions between ribozyme folding, antisense mechanisms, and the observed knockdown effect.

Target 5′-Hydrolysis

A reaction, such as 5′-hydrolysis at the G₆:uₛ site, could be occurring to cleave the target RNA [32]. Hydrolysis would not require an active ribozyme. The G:U pairing along with a folding structure that permits hydrolysis may be sufficient for target RNA cleavage. Even though ribozyme mutations may inactivate its splicing function, the ribozyme may still be able to facilitate hydrolysis of the target. The G264A ribozyme mutant decreases the rate of 5′-hydrolysis 10-fold [96]. Thus, the G264A mutant may affect knockdown not through splicing or antisense effects, but rather by affecting hydrolysis.

Evidence against this mechanism are the results for the constructs missing G₆. The most efficient trans-knockdown construct had the entire ribozyme and part of the IGS, including G₆, deleted. These results do not rule out the possibility of G:U base pairs forming elsewhere or target hydrolysis at other sites.

### Active Ribozyme

The ribozyme could possibly play a small role in the knockdown. The results with the trans-splicing ribozymes show that splicing was occurring at some low level. For trans-knockdown, only the first step of splicing, the cleavage of the target RNA, is necessary. As the ribozyme does not need to have a 3′-exon for cleavage, the ribozyme could be a true multiple turnover enzyme.

Thus, knockdown could be due to ribozymes cleaving many target RNAs. Mutations in the ribozyme and IGS generally led to less knockdown, supporting the hypothesis that the ribozyme may play a role. However, the large knockdown seen from constructs without the ribozyme indicates that the ribozyme does not contribute significantly to the knockdown.

Summary

Engineering trans-knockdown could be useful for implementing synthetic systems or for perturbing existing systems. The knockdown of around 50% for multiple targets with relatively little optimization indicates that knockdown may not be difficult to engineer.

Knockdown efficiency can perhaps be enhanced by targeting multiple sites [93], but to truly optimize efficiency, we will likely need to understand better the mechanism behind knockdown. To clarify the knockdown mechanism, future work could use primer extension to determine if the target RNA is being cleaved. If the effect is antisense-mediated, then being able to computationally model how an antisense sequence affects a target sequence would be helpful.

The sensitivity of the trans-knockdown to small sequence changes may present a challenge for engineering RNA and also be an opportunity for studying RNA structure. A single base change (e.g., the G264A ribozyme) can possibly affect the folding and function of an antisense region around 250 nt away. We may be able to use antisense-mediated effects as a reporter for studying RNA folding and structure. For example, trans-knockdown could be used as a reporter for whether ribozyme mutants are folding properly.

### Trans-Splicing Inefficiency

The trans-splicing ribozymes unambiguously show that trans-splicing is possible but inefficient. Trans-splicing has been previously shown to have efficiencies from 10% to 50% with higher efficiencies using stronger promoters [25]. However, getting above 50% splicing efficiency has been difficult, even in vitro, with long incubation times, and with excess of ribozyme [93]. Rogers et al. [126] measured trans-splicing efficiency in mammalian cells and to found an overall efficiency of 1.2% in the population. However, in single cells, 18% of the cells showed significant splicing activity, with large cell-to-cell variability. It is unknown whether the systems described here have large cell-to-cell variability.

In the experiments here, even using LacZ, an extremely sensitive reporter, the signal was barely detectable. As I calibrated the measurements using a standard reference of purified LacZ, the number of LacZα molecules per cell can be estimated at around 1-20 molecules. Although the translation efficiency is unknown, this small number of protein molecules likely corresponds to few correctly spliced RNA per cell even after overnight growth.

I discuss some possible reasons for the inefficiency of trans-splicing.

Finding the Target RNA

Cis-splicing is highly efficient, whereas trans-splicing is highly inefficient. One hypothesis for this inefficiency is that the ribozyme and the target RNA are unable to find each other. However, from the trans-knockdown results, we can estimate that roughly 50% of the target RNA can be bound by the antisense or ribozyme RNA. Thus, the inefficiency of trans-splicing is likely due to some factor other than co-localization of the target and the ribozyme.

Ribozyme Folding

Translation can help stabilize the ribozyme structure, with the ribosome unfolding incorrect pairings between the ribozyme and surrounding sequences [128, 131]. Stop codons at some 5′ splice sites can facilitate splicing. Both adding earlier stop codons or removing stop codons can lower splicing efficiency by changing the interaction between the ribosome and ribozyme. In the designed trans-splicing ribozymes, the antisense sequence was untranslated so the ribosome could not facilitate folding. The long antisense sequences may inhibit ribozyme folding by pairing with the ribozyme. We can test the role of the ribosome by adding a ribosome binding site to translate the antisense region of trans-splicing ribozymes.

Anti-IGS

I included the anti-IGS in the design of trans-splicing ribozymes due to preliminary experiments showing toxicity of ribozymes without an anti-IGS, especially in the absence of the target. Qualitative evidence from colony counting of transformations showed a twofold toxicity difference between having an anti-IGS and not having an anti-IGS. Also, transforming a ribozyme with an anti-IGS gave comparable colony counts to when an inactive ribozyme was used. This toxicity would suggest the ribozyme is capable of trans-splicing at a reasonable efficiency. In addition, for the trans-knockdown ribozymes, knockdown was dependent on the anti-IGS. However, the experimental data (FIG. 31) clearly indicates that the anti-IGS reduced trans-splicing efficiency. Eliminating the anti-IGS or reducing its binding strength showed increased trans-splicing. No obvious growth defects were seen with the ribozyme lacking an anti-IGS. Therefore, the anti-IGS should not be used in trans-splicing. Even without an anti-IGS, the amount of trans-spliced product was still low, so other factors must also contribute to the inefficiency.

3′-Exon Hydrolysis

There are side reactions, which could be favored over the correct reaction, especially as the time to splice is likely longer in trans-splicing than in cis-splicing. An especially important side reaction is the hydrolysis of the 3′-exon, which would eliminate the possibility of correct splicing. The ribozyme must find the target RNA and splice before it loses the 3′-exon. Ribozymes missing the 3′-exon can still perform the cleavage reaction and is one possible explanation for how trans-knockdown can be more efficient than trans-splicing. If the observed inefficiency from trans-splicing is due to the 3′-hydrolysis activity, then re-designing the ribozyme to not have this unwanted activity would be highly beneficial. Williams et al. [161] selected for new P5abc domains and found that the ribozymes could still splice but were deficient in 3′-hydrolysis. In another group I ribozyme, the 3′-exon hydrolysis reaction was reduced while not affecting splicing by mutating the L9.2 sequence [67]. This 3′-hydrolysis deficient ribozyme was shown to trans-splice equivalently to the ribozyme capable of 3′-exon hydrolysis [99]. Thus, a suitably engineered ribozyme may allow trans-splicing to reach the efficiencies seen with trans-knockdown.

### Conclusion

Although the trans-knockdown results indicate that a ribozyme can find a target RNA, trans-splicing was inexplicably inefficient. Trans-knockdown and trans-splicing could have many uses in the trans-rewriting of RNAs. As trans-splicing has many unique applications for synthetic biology, understanding and optimizing the reaction should be a top priority.

### Example 4

### Standardization

Choosing a splice site is necessary when designing a new ribozyme splicing system. Although the splice site must be at a U, a method is needed to select a splice site from the many potential Us. In addition, it is often straightforward to redesign an equivalent RNA sequence that contains more Us. For example, we can add Us to untranslated regions or recode coding sequences to use synonymous codons containing Us. Here, I discuss splice site selection and standardization.

### Splice Site Selection Methods

There are different methods for choosing a splice site. I discuss four criteria for evaluating splice site selection methods: functionality, efficiency, flexibility, and ease of design. Functionality and efficiency depend on the goodness of the splice site chosen whereas flexibility and ease of design are characteristic of the method itself. Using these criteria, I describe and evaluate five possible splice site selection methods.

### Evaluating Splice Site Selection

Functionality

Given an RNA X, a splice site splits X into the two RNAs X₁ and X₂. An engineered splicing system is only interesting if the unspliced and spliced states show a difference in functionality. That is, f(X) ≠f(X₁+X₂), where the functionality, f, is defined however the biological engineer wishes. For example, for an RNA encoding a protein, a logical definition for f would be the amount of translated and functional protein. If the two RNAs X₁+X₂ are translated into two peptides which come together to form a functional protein equivalent to the intact X (such as in LacZ complementation or split GFP [26, 169, 174]), then the splice site does not do a good job of splitting functionality and splicing serves no useful purpose. Thus, for engineering biological systems, splice sites should be chosen to cause a functional change. For most applications, a larger functionality difference is likely more useful.

Efficiency

A good splice site should be spliced efficiently or, at least, with a predictable efficiency. Efficient splicing partially depends on the accessibility of the splice site to the IGS. Accessibility can depend on many factors, such as the interaction of the RNA with other molecules and the folded structure of the RNA. In addition to accessibility, the IGS can to greatly affect splicing efficiency.

Flexibility

The flexibility of a splice site selection method is the freedom the designer has in choosing a site. Flexibility is not always desirable in an engineering context. For example, flexibility at the cost of possibly engineering a non-working system is not desirable. The ideal situation is to have the flexibility to intelligently choose among several possibilities.

Design Ease

Choosing a splice site should not be a chore. Reducing the amount of experimental work and thinking required simplifies design and reduces the possibility for human errors. An easy design process facilitates faster engineering and the capability for scale-up.

Unconstrained Selection

One method for choosing a splice site is to allow the designer to choose any U as a splice site. This method is highly flexible and easy for the designer. However, the properties of the splice site are indeterministic. Assuming there are more poor splice sites than good splice sites, which seems to be experimentally valid, then it becomes unlikely a good splice site will be chosen. Therefore, this method is not particularly useful unless most possible splice sites are shown to be good.

Random Selection

An efficient splice site can be found experimentally using in vitro selection. A typical method involves generating a ribozyme library with a random IGS of GNNNNN [27]. This IGS is then allowed to react with the target trans-RNA in vitro. The spliced products are isolated and sequenced to determine the splice point. This method finds the most efficient splice sites. However, the functional difference between the spliced and unspliced states is indeterminate. It may also be difficult to select for lower efficiency sites if the highest efficiency sites are not desirable for some reason. Thus, the flexibility of the method is low and the method is experimentally time-consuming

### Computationally Predicting Efficiency

An alternative to experimentally determining efficient splice sites is to computationally predict them using RNA folding methods. In simple cases, the efficiency may be able to be computationally predicted. For longer RNAs, such as in trans-splicing when the IGS and splice site are on different molecules, target accessibility is extremely important and can be predicted with tools like Sfold [136]. Just as in the random selection method, the functional effect of a splice site is not typically included in the prediction. Depending on the quality of the computational prediction, the chosen splice sites can be less efficient than found through selection. However, a computational method would allow additional flexibility in choosing splice sites of different efficiencies and is much easier than experimental selection.

### Maximal Disruption

The maximal disruption method requires looking at the target RNA and determining the point at which splicing would cause the maximal disruption of function. For example, in the case of a protein coding sequence, splicing inside a critical amino acid would be expected to completely disrupt the function of the protein. In one example, the splice site was chosen to be in the fluorophore of GFP, leading to zero background expression before splicing. This method requires knowing the points where nucleotides must be next to each other to be functional, such as when they form the codon of a critical amino acid. For inserting the ribozyme in the middle of an amino acid codon, the requirement of splicing at a U limits the amino acids that can be disrupted to cysteine, isoleucine, leucine, methionine, phenylalanine, serine, tryptophan, tyrosine, and valine (using the universal codon table). There may be many disruptive splice sites and this method allows the designer to choose among them. Although this method optimizes for splicing functionality, the splicing efficiency is indeterminate. Also, choosing a good maximally disruptive site requires a large amount of background knowledge about the target RNA. Therefore, it may not be easy for the designer, especially if the target is not well-characterized.

### Standardization

In the standardization method, a reusable module containing a standard splice site is used. A standard module allows optimizing the functionality and efficiency of a splice site once. After optimization, the same module can be used for many different constructs. Thus, the functionality and splicing efficiency are likely high and the ease of design is simple.

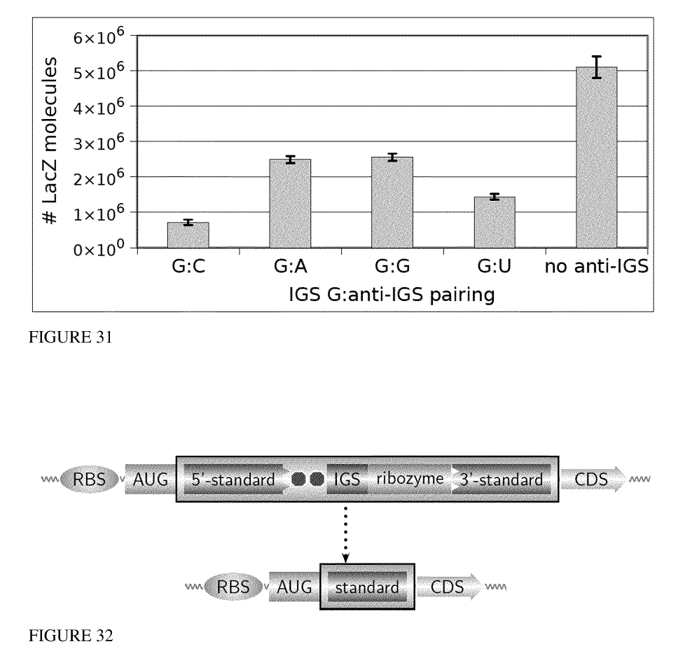

For these reasons, I chose to design standardized, reusable, functional, and efficient splicing modules. Standard splicing modules can be created for different classes of RNA targets. FIG. 32 shows a schematic for a standard splicing module designed for protein coding sequences. The boxed region is a reusable module that provides the ribozyme with a standard splicing context. Although the module can be inserted after any codon in the coding sequence, for simplicity, I will only consider inserting the module immediately after the AUG start codon. Before splicing, there would ideally be no background translation of the coding sequence. The stop codons upstream of the IGS should prevent any translation from the RBS through the ribozyme and into the coding sequence. These stop codons do not eliminate the possibility that the coding sequence itself has an internal RBS and start codon that can generate a shortened but functional protein. However, if a shortened coding sequence is translatable by itself, then we can shorten the coding sequence until we have a minimal sequence that requires a separate RBS and AUG for expression. For the maximal functionality swing, after splicing, the coding sequence should be highly expressed.

A side effect of using a standard splicing context is that the spliced protein will contain a standard leader peptide not present in the original coding sequence. Therefore, we must choose a standard leader peptide that can be attached to the N-terminus of many coding sequences without affecting their ability to function. In addition, the standard sequence can affect the efficiency of ribosome binding and translation initiation. A good module should splice efficiently. Efficient splicing depends on the sequence around the splice site and an optimized IGS. Enough standard sequence should be included in the module such that the IGS and splicing efficiency are independent of the upstream and downstream sequences. Therefore, the splicing module can be optimized once and reused in many different contexts with the same splicing behavior.

### Summary

Table 8 summarizes the five splice site selection methods and their different properties.

TABLE 8
Five splice site selection methods are rated on the functionality
and efficiency of the chosen splice site, the amount of exibility
in choosing a site, and the ease with which a site can be
chosen. For some methods, it is not possible to know
beforehand how functional or efficient a splice site will be.

| | Functionality | Efficiency | Flexibility | Design ease |
|---|---|---|---|---|
| Unconstrained | depends | depends | high | easy |
| Selection | depends | high | none | difficult |
| Efficiency | depends | medium/high | some | medium |
| prediction | | | | |
| Maximal | high | depends | some | medium |
| disruption | | | | |
| Standardization | medium/high | high | little | easy |

### Standard Ribozyme Design

Using the scheme in FIG. 32, I designed several standard modules with different sequences. Three sequences can be standardized: the standard 5′-sequence, the IGS, and the standard 3′-sequence. These sequences determine a standard leader peptide that is attached to the N-terminus of the translated coding sequence. Table 9 lists the sequences for seven versions of a standard splicing module. For testing, all versions used GFP as the coding sequence, with BBa B0034 as the RBS and driven by the BBa R0040 promoter. Versions 1-3 contained the same IGS and 3′-standard sequence. I optimized version 1 for translation efficiency after splicing. I used the bias found in the initial codons of highly expressed genes (Table 1 of Tats et al. [147]) to design the 5′-standard sequence. For the 3′-sequence, two glycines were chosen as a linker.

To test the ribosome footprint, in version 2, I increased the spacing between the RBS and the splice site by including two identical copies of the 5′-splice site. The IGS pairing with either copy should splice the coding sequence in frame with the start codon. However, computational RNA folding of the version 2 module showed that the IGS would tend to bind to the first copy of the standard sequence, so in version 3, the first copy of the splice site was modified to enhance the binding of the IGS to the second splice site.

TABLE 9

| Ver- | | 3′- | Peptide | |
|---|---|---|---|---|
| sion | 5′-sequence | sequence | linker | IGS |
| 1 | gcu acu auₛu | u ggc ggu | ATIGG | ACCGCCAG₆ |
| | ggc uaa uaa | | | UAGUA |
| 2 | gcu acu auₛu | u ggc ggu | ATIG | ACCGCCAG₆ |
| | ggc gcu acu | | [ATIG]G | UAGUA |
| | auₛu ggc uaa | | | |
| | uaa | | | |
| 3 | caa aca aua | u ggc ggu | QTIGA | ACCGCCAG₆ |
| | gga gcu acu | | TIGG | UAGUA |
| | auₛu ggc uaa | | | |
| | uaa | | | |
| 4 | gug agc aag | u aag gua | VSKGE | UACCUUUG₆ |
| | ggc gag gag | | EDNNM | GAGGG |
| | gau aac aug | | ADSLK | |
| | gcc gac ucu | | V | |
| | cuₛa aau agc | | | |
| | uaa uaa | | | |
| 5 | gac ucu cuₛa | u aag gua | DSLKV | UACCUUUG₆ |
| | aau agc uaa | | | GAGGG |
| | uaa | | | |
| 6 | gcu aaa auu | u aag gua | AKIKG | UACCUUUG₆ |
| | aaa ggu gac | | DSLKV | GAGGG |
| | ucu cuₛa aau | | | |
| | agc uaa uaa | | | |
| 7 | gca aaa gca | u aag gua | AKAKA | UACCUUUG₆ |
| | aaa gca gac | | DSLKV | GAGGG |
| | ucu cuₛa aau | | | |
| | agc uaa uaa | | | |

Seven standard splicing modules were designed using the scheme in FIG. 32. All 5′-sequences end with two stop codons (UAA). Versions 1-3 and versions 4-7 are two module families, each containing the same IGS and 3′-sequence. The intended splice site us is expected to pair with G₆ in the IGS. Version 2 contains two possible splice sites. After splicing, the
5′-sequence and 3′-sequence are translated into a peptide linker that maintains the reading frame from the upstream to downstream sequence.

Versions 4-7 form another family of standard modules containing the same IGS and 31-standard sequence. The IGS and the sequence context for splicing was taken from the native Tetrahymena sequence (FIG. 2). Version 4 contained a relatively long leader sequence to separate the splice site from the RBS. This leader sequence was derived from the initial sequence of mCherry (BBa_J06504) which was itself from EGFP [135]. In version 5, the distance between the RBS and splice site was minimized. In version 6, I added codons back to the 5′-standard sequence of version 5, optimizing again for translational efficiency using the bias seen in highly expressed proteins [147]. Highly expressed proteins have increased frequency of alanine in the second amino acid and especially the GCN codon, so I used GCU as the codon to immediately follow the AUG start codon. The third codon is biased for lysine, and generally RNAs rich in As near the start codon lead to unstructured RNA and perhaps better translation, so I chose AAA. The fourth codon is biased for isoleucine and the fifth for lysine, so appropriate codons were chosen. Finally, a glycine was to inserted as a linker between this leader sequence and the amino acids arising from the native Tetrahymena sequence. I designed version 7 by replacing the initial Us in version 6 so that there would be fewer Us for off-target splicing.

### Experimental Design

All constructs were on the plasmid pSB1A3. I used GFP, mCherry, LacZα, KanR, and ATF1 to characterize the standard splicing modules. For GFP, KanR, and ATF1, I inserted the standard splicing module between the initial AUG start codon and the rest of the sequence. For mCherry, amino acids 1-9 were first deleted and then the standard module was inserted after Met10. This deletion eliminates the possibility of translation initiation from a likely internal RBS and Met10.

An unintended point mutation in mCherry at codon 11 (AlaThr) was found during sequencing. For LacZα, the first two amino acids were deleted and the standard module inserted after Met3. For each reporter, an inactive splicing module with a G264A ribozyme mutant served as the unspliced control. I took GFP measurements by growing single colonies in a plate reader and measuring the maximum synthesis rate. mCherry and LacZα expression levels were characterized as described elsewhere herein.

For each colony, I subtracted the average background activity from cells not expressing the reporter. After background subtraction, the data was normalized using a reference intact reporter expressed from the same promoter and RBS. To test ATF1 expression, I grew cultures overnight in 10 ml LB with 4.4 μl isoamyl alcohol. A constitutively expressed ATF1 (BBa J45200) served as a positive control. After growth, the culture odor was determined by smell. Although non-quantitative, there was no ambiguity from the smell test whether the cultures were expressing ATF1 (banana smell) or not.

### Designing a Standard Splicing Module

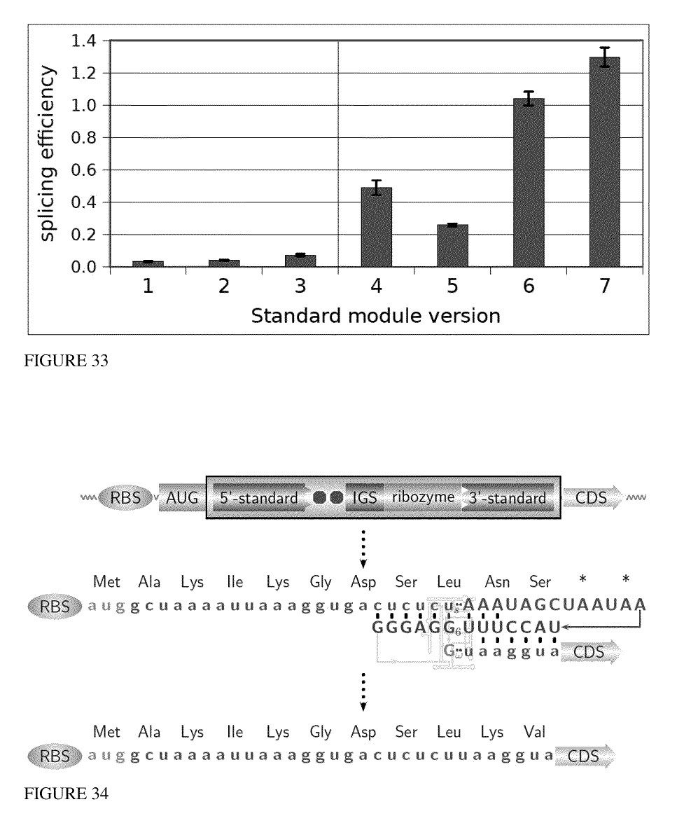

FIG. 33 shows the efficiencies for the standard splicing modules in Table 9. Standard module versions 1-3 had negligible splicing, whereas versions 4-7, using the native Tetrahymena sequence context, showed reasonable splicing efficiencies.

### Testing the Standard Module

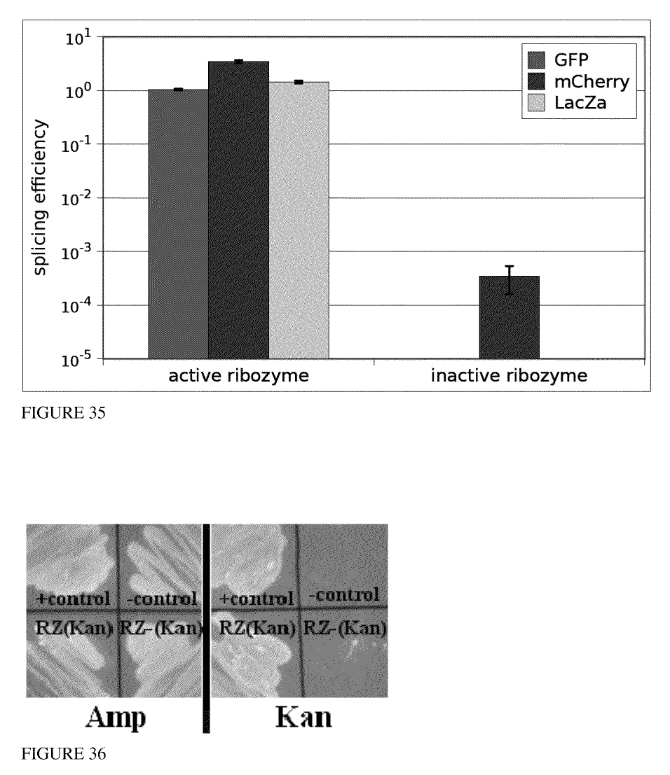

I tested the modularity of the version 6 splicing module by using either GFP, mCherry, LacZα, KanR, or ATF1 as the coding sequence (FIG. 34). Using GFP, mCherry, or LacZα as the coding sequence, the reporter activity went from background, with an inactive ribozyme, up to a level exceeding that of the original reporter, with an active ribozyme (FIG. 35). FIG. 36 shows antibiotic resistance tests for cells containing KanR with the splicing module. All cells were ampicillin resistant as the plasmids contain ampicillin resistance.

Additionally, an active splicing module conferred kanamycin resistant, whereas cells with an inactive ribozyme were not able to grow on kanamycin. Thus, even with selective pressure, cells with a nearly complete kanamycin resistance gene could not survive when splicing was inactivated. Finally, I tested the standard module with alcohol acetyltransferase I (ATF1) (BBa_J45014) (Payne et al., submitted). ATF1 converts isoamyl alcohol to isoamyl acetate that has a distinctive banana-like smell. Cells containing an active splicing ATF1 had a distinctive banana smell, indistinguishable from cells with the intact ATF1. On the other hand, cells with ATF1 and an inactive splicing module had no smell distinguishable from normal E. coli.

### Standard Splicing Module

I tried several versions of a standard splicing module and many failed to splice efficiently (FIG. 33). Versions 1-3 all contained the same synthetic IGS and standard 3′-sequence and showed low splicing efficiency.

One explanation is that the ribosome footprint while bound to the RBS prevented proper folding required for splicing. Another explanation is that the designed IGS was inefficient for splicing, perhaps because the P1 pairing was too strong. The P1 helix can possibly form 11 bp, which may not allow for dissociation and formation of the P10. A third, less likely, possibility is that splicing was efficient, but that translation was inefficient due to the standard sequence chosen.

To eliminate the possibility of inefficient catalytic activity, for versions 4-7, the IGS and splice contexts were based on the native Tetrahymena sequence. Presumably, the native context is highly efficient as the ribozyme must splice out of the essential ribosomal RNA. Version 4, containing a long leader sequence, showed significant splicing activity, but still only around 50% of the activity of intact GFP. Part of this inefficiency may be due to an unintended RBS and start codon in the leader sequence, thus again introducing competition between the ribosome and ribozyme. Version 5 directly tested the hypothesis that competition with the ribosome lowers splicing efficiency. Version 5, with a minimal distance between the RBS and splice site, showed an efficiency drop relative to version 4. Interestingly, even though the splice sites in versions 1 and 5 were at the same distance from the RBS, splicing was significantly higher for version 5 using the native sequence context.

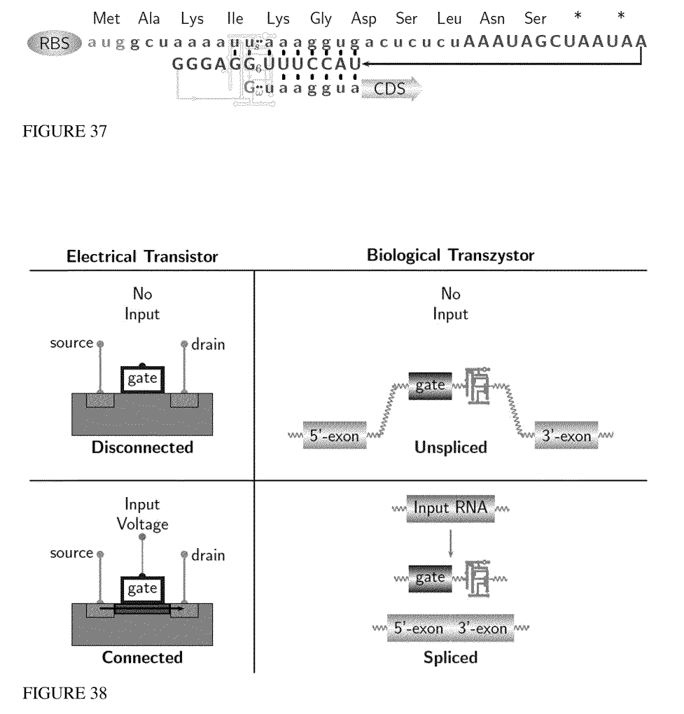

Thus, efficient splicing requires both a good IGS and enough spacing to prevent conflict between the ribosome and ribozyme. Version 6 had additional spacer sequence relative to version 5, optimizing for translational efficiency after splicing. Results with the version 6 module showed that the splicing activity was equivalent to the intact reporter. In effect, the ribozyme spliced itself out so efficiently, that it was as if it were not there. After completion of most experiments, computational folding of the version 6 module detected a second possible pairing between the IGS and the 5′-standard sequence (FIG. 37). This alternative pairing also forms a G₆:U base pair. If spliced at this site, the coding sequence would not be joined in frame, leading to an untranslated coding sequence.

To prevent the alternative splicing, in version 7, I changed the leader sequence so that it contained no extra Us between the start codon and the correct P1 region. Although the standard leader peptide in version 7 was slightly different than version 6, the difference was not expected to dramatically change the translation efficiency. Results with the version 7 module showed activity higher than the base reporter, possibly due to both efficient splicing and increased translational efficiency.

### Functional Composability of a Standardized Module

The five reporters GFP, mCherry, LacZα, KanR, and ATF1 all worked as expected when attached to the version 6 standard splicing module. They functioned across diverse measurement techniques: fluorescence, antibiotic resistance, and smell test. The three reporters that were quantitatively characterized (FIG. 35) showed that the activity with the splicing module was greater than the original reporter. Even higher activity may be possible if the more efficient version 7 splicing module had been used instead. With mCherry, the splicing module showed significantly higher activity than the original reporter and showed much higher variability than any other construct. Other than the ribozyme, there are three sequence differences between the original mCherry and the expected spliced mCherry. First, the first nine amino acids were removed from the original mCherry to eliminate a possible internal translation start site. These amino acids are not expected to affect the fluorescence as they were originally added to mCherry to enhance tolerance to protein fusions [135]. Secondly, an unintended mutation (Alal lThr) could have affected fluorescence. Finally, the spliced RNA contained a leader sequence. This leader sequence was chosen to enhance translation, so perhaps it enhances the translation of mCherry more than the other reporters.

### Module Optimization

I have demonstrated a standard splicing module that is efficient and functionally composable across a range of coding sequences. To avoid optimizing the IGS for efficient splicing, I used the native Tetrahymena context. However, different sequence contexts, such as used in versions 1-3, may require IGS optimization. The advantage of a standard module is that we can optimize once and then reuse the module in different contexts. For example, we can screen for high efficiency splicing modules using an IGS library with KanR as the coding sequence. After optimization, KanR can be replaced by a different coding sequence, without having to re-optimize. Additional modules can be designed and optimized for different applications, such as for non-coding sequences.

### Conclusion

Many advantages come from standardizing splice sites and creating functionally composable splicing modules. The entire splicing process becomes independent of any upstream or downstream sequence such as the promoter, the RBS, or the coding sequence. Modularity allows us to optimize once and reuse often. In addition, the design of splicing systems is split into two independent tasks: choosing a standard splicing module and choosing the target sequence to be spliced. Standardization removes most of the thought required for engineering splicing. The design and construction of the five splicing reporter systems was extremely easy, highlighting a major reason to use composable modules.

A standard splice site was shown to be highly functional and efficiently spliced across many reporters. When splicing was inactivated, there was no activity, whereas the activity from the spliced construct was as high as the original reporter. With this large dynamic range, we can begin to engineer splicing control. For example, we can change splicing efficiencies by manipulating the internal guide sequence or we can regulate splicing. Although standard modules can be applied to both cis- and trans-splicing, I have only considered the cis-splicing case here due to the experimental ease.

Standard modules could also be designed for trans-splicing where a module is split into two parts: one to be attached downstream of the 5′-exon and one to be attached upstream of the 3′-exon. However, in some applications such as trans-knockdown, the target may be fixed, ruling out attaching a standard sequence. Except for situations where the designer does not have full control over the RNA sequences, standard splicing modules provide clear engineering benefits and should be used when possible.

### Example 5

### Transzystors

Transistors are the basic building blocks of electronics. A transistor is a simple switch, but its ability to endogenously regulate the flow of electrons makes it a powerful component. Similarly, being able to regulate RNAs using endogenous biological components would be extremely powerful. Here, I discuss the design and implementation of a biological transzystor based upon the splicing ribozyme (FIG. 38). The term “transzystor”, accordingly, refers to a biological component that can act as part of a synthetic biology logic, at least in some aspects, in analogy to the way a transistor acts in an electronic logic. Some conditionally active ribozymes, as provided by some aspects of this invention, can fulfill this role as described in detail herein. Accordingly, the term “transzystors” also refers to such ribozymes.

In support of an all-RNA logic, the input, output, and the transzystor device are RNA. The gate of the transzystor detects a trans-input RNA and regulates splicing. Without the input, the gate inhibits splicing, leaving the two exons unspliced (“off” state). With the input, the gate allows the ribozyme to splice, producing a spliced output RNA (“on” state). Thus, the transzystor implements an RNA switch. However, unlike transistors which are reversible, transzystors can only switch in one direction. Once spliced, transzystors cannot switch back to the unspliced state.

### Previous Work

There has been much work in designing RNA switches that can sense small molecules, proteins, and oligonucleotides [140]. Although not many switches have been demonstrated to function in vivo, there are several examples of in vivo RNA detection using transzystor-like devices. The transzystor is similar to a split reporter system, also based on the Tetrahymena ribozyme [65], where the ribozyme is split at the L1 loop, between the IGS and 5′-exon. The two fragments are brought together by pairing with a third target RNA. Thus, only when the target is present would the ribozyme splice the exons together. In effect, this system relies on the inefficiency of the trans-splicing reaction without an antisense region. The target RNA base pairs with both the ribozyme and the 5′-exon bringing them together and facilitating splicing. This system requires three RNAs to come together and is not efficient in practice (personal communications).

Another RNA control system are the riboregulators designed by Isaacs et al. [77]. These riboregulators control translation initiation using a cis-repression sequence that binds to the RBS, preventing translation. Translation is activated by a trans-RNA that unbinds the repression sequence. The riboregulator design only requires two RNAs to come together. However, the trans-activating RNA is a designed sequence and dependent on the RBS sequence. For transzystors, I assume that both the input and output sequences are given.

### Transzystor Design

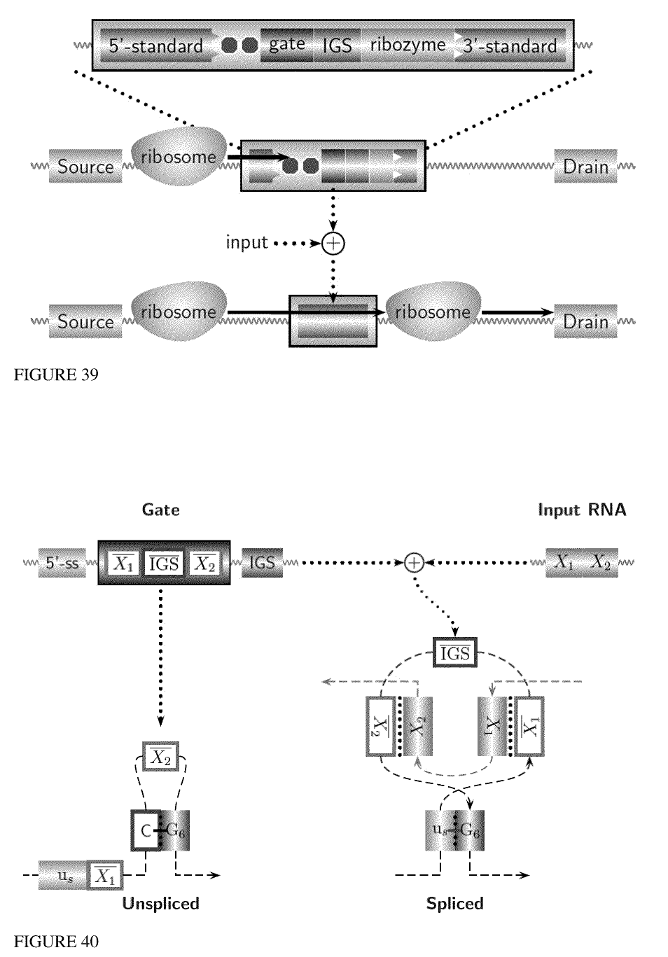

An ideal transzystor would have a tight “off” and a high “on” state. Thus, efficient switching requires an optimized IGS for maximum possible splicing and a gate that inhibits splicing in the absence of an input RNA. I built transzystors using highly efficient standard splicing modules. FIG. 39 shows the standard transzystor design scheme. A gate module between the IGS and the 5′-splice site regulates the splicing of an output coding sequence.

The information flow in these standard transzystors is analogous to that in electrical transistors. Whereas transistors regulate the flow of electrons (current), transzystors instead regulate the flow of ribosomes. The upstream source region of a transzystor generates ribosomes that fall off at the stop codons in the unspliced state. Only in the spliced state are the ribosomes able to flow from the source to the downstream drain region. Thus, ribosome flow is dependent on the input RNA.

### Methods

All transzystors were on the high-copy plasmid pSB1A3. For driving input levels, gfp or mcherry was expressed from the inducible promoter BB a_F2620 [29]. The gfp input was on the plasmid pSB3K3 and the mcherry input was on pSB4K5. BBa_F2620 was induced with 3-oxoctanoyl-homoserine lactone (Sigma Aldrich #O1764), called AHL here.

### Transzystor Gate Design

FIG. 40 shows the design of a transzystor gate that detects an input RNA. It contains an anti-IGS region that base pairs with the IGS, sequestering the critical G₆ in a G:C base pair and preventing splicing. The gate also contains two regions of antisense pairing with the input RNA. When the input RNA pairs with the antisense regions, the anti-IGS is pulled away from the IGS, allowing the IGS to pair with the 5′-splice site and activating splicing. The order of the two antisense regions is flipped relative to the input RNA to allow the IGS and 5′-splice site to be near each other upon binding of the input. If the two antisense regions were not flipped, then the IGS and 5′-splice site would be pushed further apart upon formation of the RNA helix between the gate and input.

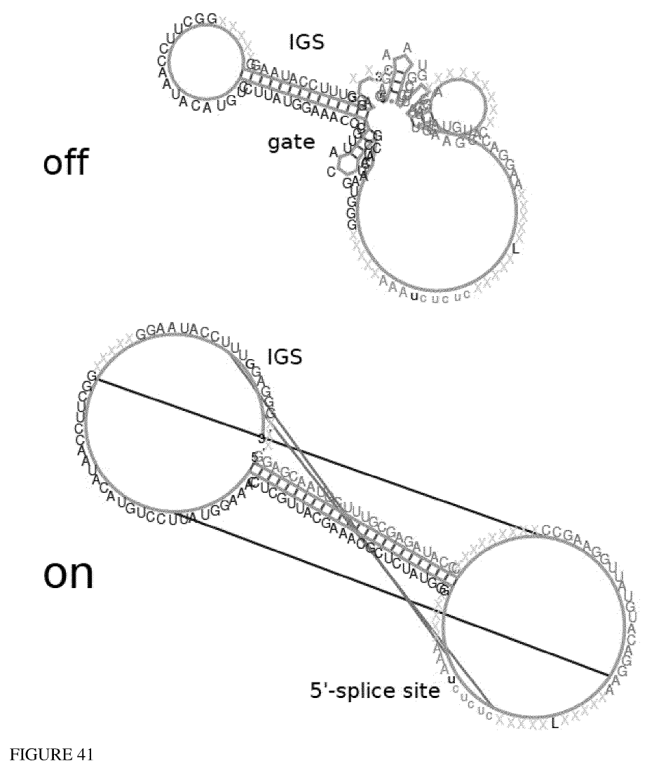

Using the kinefold program [165], I simulated the folding of a simplified transzystor (FIG. 41). The input and the transzystor RNAs were concatenated with a special L base separating the two RNAs. The L tells kinefold to fold the two RNAs separately for the first third of the time. In the remaining time, the two RNAs are allowed to fold together. The Xs are ignored by kinefold and used as a spacer sequence. The simulation was run for a total of 15 simulated seconds. In the first third of the simulation when the input RNA does not affect the transzystor folding, the IGS should base pair with the anti-IGS in the gate and not with the 5′-splice site. After the input RNA is allowed to interact with the transzystor, the IGS should switch to base pairing with the 5′-splice site instead of the anti-IGS. The simulated folding indeed shows this switching behavior, providing evidence for proper functioning of the gate.

### Transzystor Variants

I constructed several transzystor variants with gfp as the input and one transzystor with mcherry as the input. All transzystors had an output of LacZα. The transzystors had gates with different lengths for the first antisense region, the anti-IGS region, and the second antisense region. In addition, the 3′-end of the anti-IGS sometimes overlapped with the second antisense region. Table 10 shows the lengths of these gate regions for the transzystor variants. All transzystors except gfp-7 used the version 6 standard splicing module (Example 4) as a base. The gfp-7 transzystor contained the same gate as the gfp-1 transzystor but used the version 7 standard splicing module. When not specified in the text, experiments with a gfp transzystor used gfp-1.

### Transfer Curves

I co-transformed the gfp-1 and mcherry transzystors with either inducible gfp or mcherry input. I measured transfer curves for these four combinations by varying the levels of the AHL inducer. Individual colonies were grown overnight in EZ media with 1 mM IPTG and concentrations of AHL ranging from 0 M to 10⁻⁵ M. For each run, measurements of the input and output at 0 AHL were used as the background and subtracted from all points. To allow comparison of arbitrary fluorescence values, the input and output measurements were normalized. For each measurement type and construct, e.g., all LacZ measurements for the gfp transzystor with mcherry input, the mean of the entire data set was subtracted from each data point. Then each point was divided by the square root of the sum of squares of all the data points. The resulting normalized data set had an average value of zero and a sum of to squares of one.

TABLE 10
The transzystor variants had different lengths (in nt) for the gate
components. All transzystors had lacZα as the output. The
first transzystor responded to mcherry RNA whereas the other
seven transzystors took gfp as an input. All transzystors except
gfp-7 used the version 6 standard splicing module (Example 4).
The gfp-7 transzystor had the same gate as gfp-1 but used the
version 7 splicing module.

| Variant | Antisense 1 | Anti-IGS | Overlap | Antisense 2 |
|---|---|---|---|---|
| mcherry | 60 | 12 | 3 | 20 |
| gfp-1 | 61 | 12 | 3 | 20 |
| gfp-2 | 61 | 14 | 3 | 20 |
| gfp-3 | 61 | 14 | 5 | 20 |
| gfp-4 | 61 | 16 | 7 | 20 |
| gfp-5 | 61 | 13 | 0 | 13 |
| gfp-6 | 50 | 12 | 3 | 20 |
| gfp-7 | 61 | 12 | 3 | 20 |

### Input Loading

The four transzystor data sets used for the transfer curves were also used to calculate the input loading effect. The background subtracted data from the gfp input constructs were pooled and normalized by the square root of the sum of squares of all the data points. Similarly, the mcherry data were pooled and normalized. After normalization, the four data sets were simultaneously fit to the Hill function

$${F}_{max} \cdot \frac{{[ AHL ]}^{n}}{{K}^{n} + {[ AHL ]}^{n}}$$

(Equation 9)

where n and K were assumed to be an intrinsic property of the inducible promoter, BB a_F2620, and thus were fixed across the four data sets. Each data set had its own Fₘₐₓ fit value. I calculated the input loading effect for the gfp transzystor as the ratio of Fₘₐₓ for the gfp input with the gfp transzystor to Fₘₐₓ for the gfp input with the mcherry transzystor. Similarly, the loading effect for the mcherry transzystor was the ratio of Fₘₐₓ for the mcherry input with the mcherry transzystor to Fₘₐₓ for the mcherry input with the gfp transzystor. A ratio of one indicates no loading effect, a ratio greater than one indicates that the transzystor increases the expression of the input, and a ratio less than one indicates a reduction of the input expression.

### Leakiness, Low, and High States

I used cells containing singly transformed transzystors to measure the leakiness due to input-independent splicing. I measured the flow and high states of a transzystor with an input by adding either 0 M AHL (uninduced flow input) or 10⁻⁵ M AHL (induced high input). Transzystors with the G264A inactive ribozyme served as splicing controls.

### Transfer Curves and Specificity

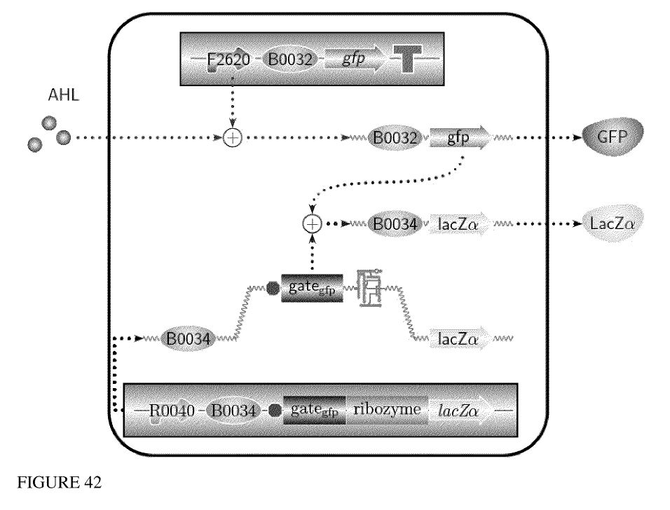

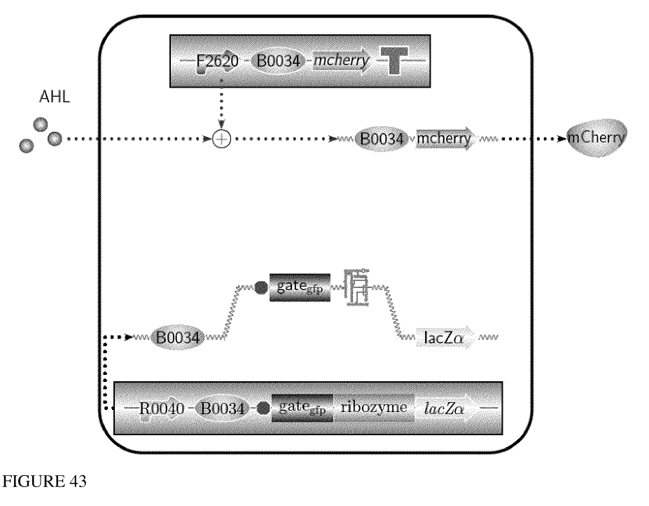

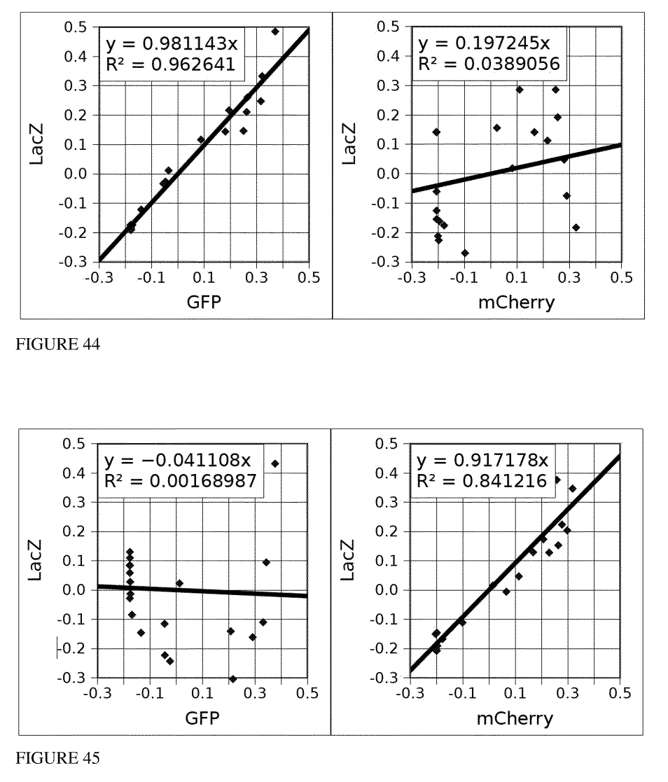

I designed a transzystor (gfp-1) using the scheme in FIG. 40 with an input of gfp and an output of LacZα. FIG. 42 shows the measurement system where this gfp transzystor was co-transformed with an inducible gfp input module. With varying inducer levels, the transzystor output, as measured by LacZ activity, should be correlated with the transzystor input, as measured by GFP fluorescence. In addition, to test specificity, the transzystor was co-transformed with an inducible mcherry module (FIG. 43), and the LacZ activity should be uncorrelated with mCherry fluorescence. FIG. 44 plots the normalized input and output activities for the gfp transzystor with the gfp or mcherry input.

If the output is a linear function of the input, then after the normalization procedure, the output will be equal to the input (slope=1). With the gfp input, there is a strong correlation between the input and output with a slope near one, indicating a linear relationship between the prenormalized GFP and LacZ levels. On the other hand, with the mcherry input, the input mCherry and output LacZ are uncorrelated, showing the specificity of the gfp transzystor response. FIG. 45 shows a second transzystor designed to respond to mcherry. Again, the transzystor showed a linear and specific response to the matched mcherry input.

### Input Loading

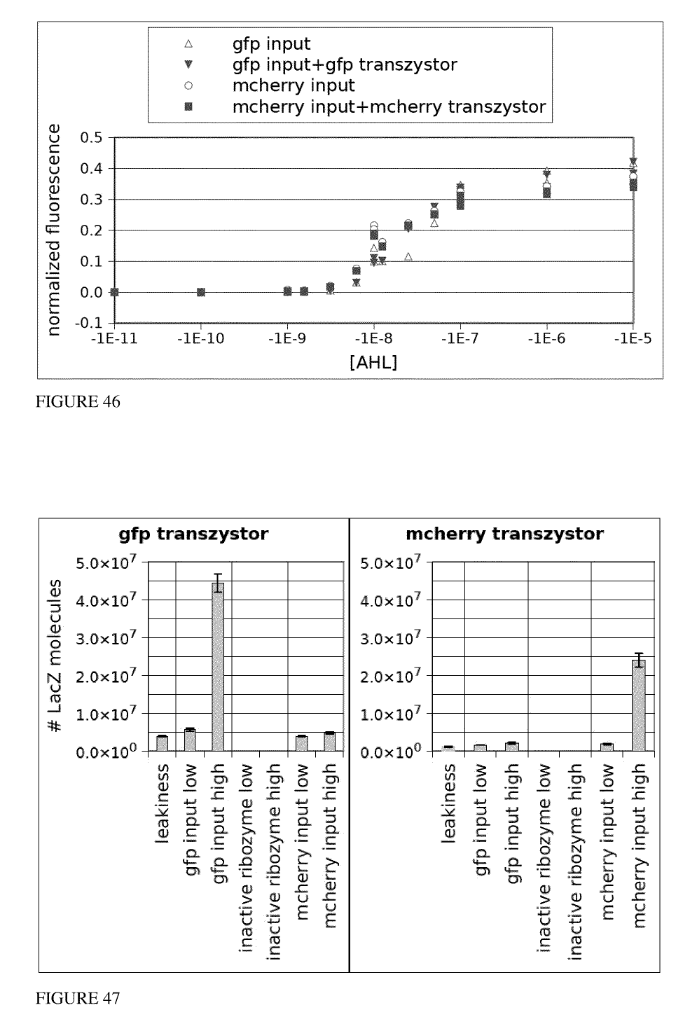

Input loading is the effect of the transzystor on the input. An ideal transzystor would not affect the input RNA level in the process of detection. However, the antisense region in the transzystor could possibly knockdown the input RNA. The combined antisense regions in the gfp transzystor was the same 81 nt antisense sequence found to be highly efficient for gfp knockdown. To quantify the loading effect, I compared the fluorescence of the input module when transformed with a transzystor containing a matched antisense region to a transzystor without a matching antisense region (FIG. 46).

The four data sets were simultaneously fit to a Hill function (Equation 9), with fit values of n=1.1 and K=10^{-7.7}. All four had an R²>0:95, indicating good fits. Based on the fit Fₘₐₓ values, the loading effect ratio for the gfp transzystor was 1.03 and the loading effect ratio for the mcherry transzystor was 0.96. As these ratios were near one, neither of these transzystors significantly affected input expression.

### Leakiness, Dynamic Range, and Sensitivity

To further quantitatively characterize transzystors, I made several additional measurements for each transzystor. The leakiness of a transzystor was measured as the output LacZ activity from the transzystor without any input. With an input, I measured the flow and high states by either not inducing the input or strongly inducing the input.

The ratio of the high state to the leakiness is the dynamic range and the ratio of the flow state to the leakiness is a measure of the sensitivity. The sensitivity ratio measures the transzystor response to basal expression from the inducible promoter BBa_F2620.

An ideal transzystor would have flow leakiness, high dynamic range, and high sensitivity. FIG. 47 shows the leakiness, low, and high states of the previous gfp and mcherry transzystors with different inputs. LacZ activity is reported using an absolute reference standard. Transzystors with an inactive ribozyme and the matching input showed no LacZ activity for either the flow or high states. Thus, all measured activity for these transzystors was due to ribozyme splicing. Table 11 shows the dynamic range and sensitivity ratios calculated from the data in FIG. 47.

Both the gfp and mcherry transzystors had a much higher dynamic range (10-20) with the matched input than with the mismatched input (dynamic range around one). With matched inputs, the mcherry transzystor had a lower high state than the gfp transzystor, but the lower leakiness lead to an overall higher dynamic range for the mcherry transzystor.

The specificity ratio quantifies the preferential response of a transzystor for the matched input over the mismatched input. Due to the higher non-specific activity of the mcherry transzystor, both transzystors showed about the same tenfold higher response for the matched input over the mismatched input. With uninduced inputs, both transzystors had a slightly higher sensitivity ratio for the matched input over the mismatched input, supporting the hypothesis that the transzystors can detect extremely flow levels of input RNA.

An ideal transzystor would have a dynamic range and sensitivity ratio of one for mismatched inputs. Only the gfp transzystor with an uninduced mcherry showed this ideal to behavior. The mcherry transzystor responded even to an uninduced gfp input. As inducing the gfp input did not significantly activate the mcherry transzystor, it is unlikely that the transzystor responded significantly to gfp. The basal activation of the mcherry transzystor may have been due to a constitutively expressed RNA on the input plasmid (e.g., the antibiotic resistance gene).

However, both transzystors showed a slight increase in activity when the mismatched input was induced as opposed to uninduced. In the induced state, the large amount of extra RNA in the cell likely increased the probability of erroneous switching of the transzystors. Thus, both of the transzystors showed detectable response to the mismatched input and had some non-specific activity at high RNA levels.

TABLE 11
The dynamic range and sensitivity ratios are shown for
the gfp and mcherry transzystors using the data in FIG. 47.

| | |
|---|---|
| gfp transzystor | mcherry transzystor |

Dynamic Range

| | | |
|---|---|---|
| gfp input | 11.53 ± 0.77 | 1.84 ± 0.29 |
| mcherry input | 1.24 ± 0.09 | 21.58 ± 0.12 |
| specificity ratio | 9.31 ± 0.79 | 11.71 ± 1.88 |

Sensitivity

| | | |
|---|---|---|
| gfp input | 1.47 ± 0.12 | 1.42 ± 0.11 |
| mcherry input | 1.01 ± 0.05 | 1.67 ± 0.21 |

(a) The dynamic range is calculated as the ratio of the high state (induced input) to the leakiness (no input). The specificity ratio is the ratio of the dynamic range for the matched input to the mismatched input.
(b) The sensitivity is calculated as the ratio of the low state (uninduced input) to the leakiness (no input).

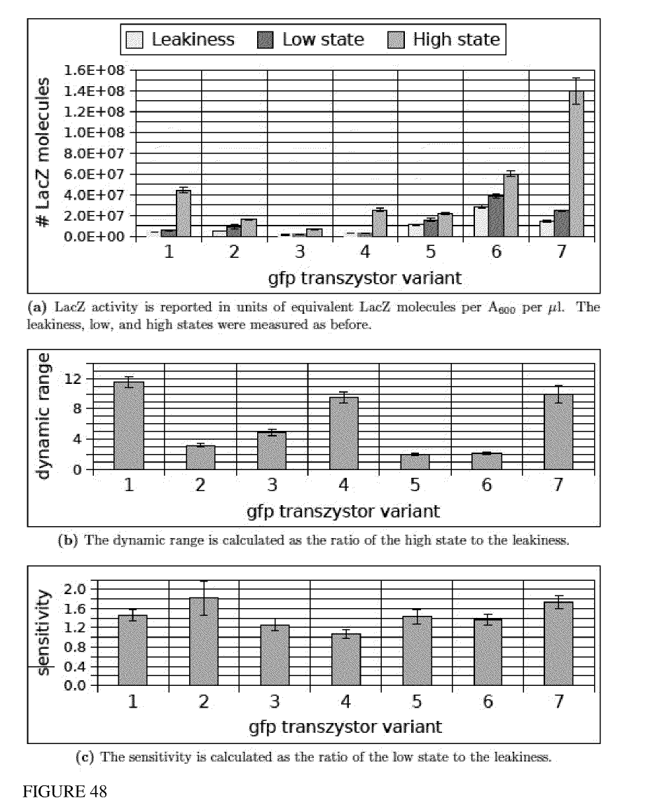

I constructed several additional gfp transzystors to test different gates and splicing modules. FIG. 48 shows the leakiness, low, and high state measurements for these variants using a matched gfp input. Variant 1 is the just described gfp transzystor (gfp-1) and is shown for comparison. The gfp-2 transzystor had a longer anti-IGS and a decreased high state. Unexpectedly, both the leakiness and flow state were higher than gfp-1. The gfp-3 transzystor to had the same anti-IGS length as gfp-2, but also contained a longer overlap between the anti-IGS and the antisense region.

A longer overlap is expected to increase the dynamic range as the binding of the input RNA should help displace the anti-IGS binding. The gfp-3 transzystor showed the expected behavior in having reduced leakiness and a higher dynamic range than gfp-2. The gfp-4 transzystor had an even longer anti-IGS and overlap compared to gfp-3. The dynamic range of gfp-4 was further increased above gfp-3. The gfp-5 variant had a shortened second antisense region and was missing the overlap region. As expected, both of these effects reduced the dynamic range significantly. The gfp-6 transzystor had a shortened first antisense region and showed an unexpected higher activity for all measurements. The gfp-7 variant contained the same transzystor gate as gfp-1 but used a higher efficiency standard splicing module. The leakiness, low, and high state measurements of gfp-7 were all about threefold greater than gfp-1. Thus, the dynamic range and sensitivity did not change much, indicating that these ratios may be relatively good metrics for characterizing transzystor gates independent of the splicing module.

### Transzystor Properties

Linear Response

The output responses for two transzystors were linear over a large input range that to likely encompasses the biologically relevant range of RNA levels (FIG. 44 and FIG. 45).

The linear response is a good indicator that the reporter proteins are an accurate reflection of the RNA levels. Although non-linearity is needed for implementing digital logic, a linear response is useful for measurement or conversion applications where the output level should reflect the input level. Further work should validate transzystors in accurately quantifying the amount of input RNA.

### Specificity

The gfp and mcherry transzystors were specific for their designed input and did not respond to a mismatched input, demonstrating two orthogonal transzystors that respond to their own input and not to the input of the other transzystor.

### Low Input Loading

The transzystors did not significantly affect the input RNA even though they contained two antisense regions (FIG. 46). It is surprising that the antisense regions used in the gfp transzystor did not show any effect when the same antisense sequence showed substantial trans-knockdown. Perhaps the gate structure prevented strong binding of the antisense to the target RNA. Like an electrical ammeter which can be inserted into an existing circuit to measure current without substantially changing the circuit being measured, the flow input loading makes transzystors an excellent tool for hooking into a biological system and measuring RNA levels without perturbing the system.

### Dynamic Range

The dynamic range in these first generation transzystors, with little optimization, was around 10-20. In the split ribozyme system of Hasegawa et al. [65], there was a 7-24 fold change in the presence of a target, comparable to the results here. To increase the dynamic range, either the leakiness can be reduced or the high state can be increased. It is unknown how far the leakiness levels can be reduced without completely inhibiting splicing. However, the LacZ expression in the high state was relatively flow and there are likely optimizations to increase the high state.

Sensitivity

The input reporters were expressed from the inducible promoter BBa_F2620. With no inducer, the fluorescence from the reporters were indistinguishable from background. However, there was probably leaky expression from an uninduced BBa_F2620 as having a complete off is unlikely. In fact, both the gfp and mcherry transzystors could detect their matched inputs even in the uninduced state. Thus, transzystors appear to be extremely sensitive and able to measure the level of an RNA which was undetectable using standard fluorescent reporters.

Scalability

The transzystor design is scalable to most any input and output. Designing a new transzystor gate is relatively simple. However, the results from the transzystor variants show that further work is needed to understand how to rationally engineer gates for specific parameters, e.g., high dynamic range. The development and validation of a computational folding model for transzystor gates similar to the IGS folding model described elsewhere herein would greatly increase the ease of designing transzystors. Penchovsky and Breaker [117] validated, in vitro, the use of computational secondary structure

Modularity

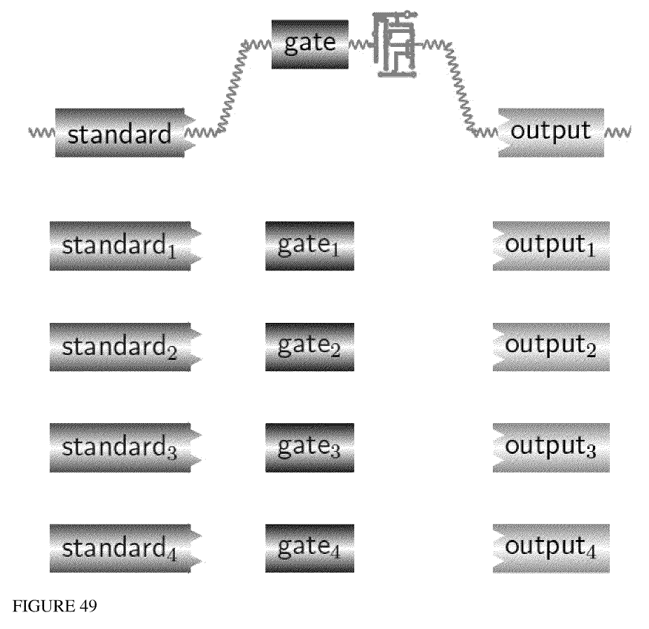

Transzystors are highly modular, as they are built upon standard splicing modules (FIG. 49). The input gates can be swapped while maintaining the same standard splicing and output modules (e.g., the gfp and mcherry transzystors). The standard splicing module can also be swapped if the IGS is maintained (e.g., the gfp-1 and the gfp-7 transzystors). The output module can be swapped and even the ribozyme core can be independently replaced as outlined elsewhere herein. Thus, due to the highly modular design, standard splicing modules, input gates, ribozymes, and output coding sequences can be independently engineered and assembled into transzystors. This modularity allows us to, for example, select for a good input gate sequence using an antibiotic resistance gene as the output and then move the gate into a transzystor with a different output.

### Multi-Transzystor Systems

To connect multiple transzystors together, the input and output RNA levels would need to be matched. The current transzystors are far from the efficiency needed for directly connecting multiple devices together. Using the gfp transzystor, I estimated the absolute number of input GFP and output LacZα molecules using standard curves of purified GFP and LacZ. Even considering that GFP may be more stable than LacZα and the inefficiency of LacZ complementation, there are likely four orders of magnitude more input GFP molecules than output LacZα molecules. Thus, these transzystors function as attenuators.

Some possible functions for attenuators include lowering power use (cellular load) and for level matching. To avoid the attenuation effect, either intrinsically better transzystors need to be developed or amplification is required. External amplification is possible such as producing a transcriptional activator as the transzystor output. The activator could then amplify the output to a level comparable to the input. However, an ideal transzystor would have built-in amplification. For example, one input RNA could activate multiple transzystors if the gate is designed such that an input RNA only binds briefly before being released for further binding with other gates. Multiple turnover of an antisense probe has been shown previously in a designed RNA switch [166]. Further research is needed to find an efficient mechanism for implementing intrinsic amplification for transzystors.

### Logic Gate Design

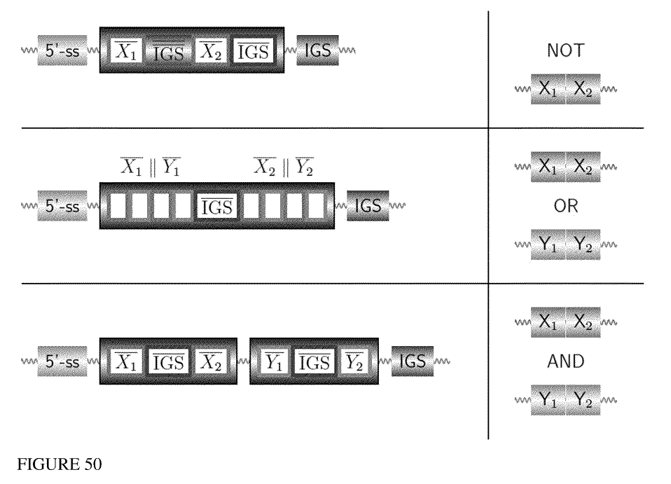

The current transzystor gate design performs a simple detection operation where splicing occurs if the input is present. However, with different gates, we can engineer transzystors to have additional functions. I present some gate designs in FIG. 50 to implement the basic logic operations. All of these gates are based on the current working gates but future work is necessary to implement and evaluate whether these more complex gates function as expected.

### NOT Gate

In a NOT gate, splicing should occur only in the absence of an input. In the NOT gate, the regulated pairing is not between the IGS and 5′-splice site but rather between the IGS and an introduced anti-IGS region. The accessibility of the anti-IGS to pair with the IGS is controlled by an anti-anti-IGS. Some care is required to ensure that the anti-anti-IGS does not pair with the 5′-splice site. In the absence of the input, the anti-anti-IGS sequesters the anti-IGS, allowing the IGS to find the splice site. The input pulls away the anti-anti-IGS, allowing the anti-IGS to sequester the IGS.

As a step towards evaluating the feasibility of this gate design, I constructed a prototype NOT gate without any antisense regions and experimentally confirmed that splicing is active. That is, the anti-anti-IGS can sequester the anti-IGS such that splicing can occur. Ensuring good repression in the presence of a target may be more difficult as cis-splicing is fast, efficient, and irreversible, whereas trans-repression is slow, inefficient, and could be reversible.

To make the NOT gate irreversible for both states, the anti-IGS could include a decoy splice site instead of just sequestering the IGS with a G₆:C base pair. Thus, once the input RNA binds and switches the gate off, splicing at the decoy state will ensure that the gate cannot turn on even when the input RNA unbinds.

### OR Gate

For an OR transzystor gate, splicing occurs if either of two inputs is present. One implementation for an OR gate is to take gates for the inputs X and Y and interleave the antisense sequences. With sufficiently long antisense regions, either input can pair with half of the antisense sequence, pulling off the anti-IGS to allow splicing.

### AND Gate

In an AND gate, splicing is dependent on two inputs being present. One implementation would be to have two consecutive gates, where each gate responds to one of the two inputs. For the IGS to find the 5′-splice site, it needs to get by two anti-IGS sequences. With sufficiently flexible linkers between components, each half of the AND gate should function similarly to the gates used here. In addition, assuming this design is feasible, other than efficiency limitations, any number of gates could be assembled together to create n-input AND gates.

A more complex design could allow for cooperative binding, as seen in some natural riboswitches [100]. Cooperativity allows for a non-linear response, which may serve as a foundation for digital logic.

### Applications

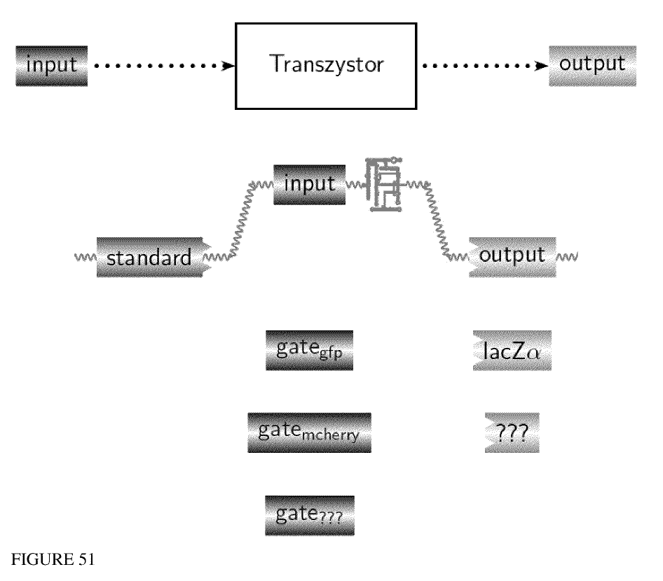

There are many possible applications for transzystors. One application is as universal RNA unit converters in synthetic circuits (FIG. 51). For example, if one device has an output of RNA X but another needs an input of RNA Y, a transzystor can convert X to Y. Thus, transzystors serve as universal wiring between systems where any RNA can serve as a signal carrier. Having genetic control over measuring RNA levels allows using intracellular RNAs to trigger an appropriate response. For example, detecting the prostate specific antigen (PSA) RNA outside of the prostate is a sign of cancer [124]. Transzystors allow the in vivo detection of such RNAs and to activate a response. Thus, transzystors serve as general RNA sensors. These sensors may find use in human gene therapy, where it would be helpful to activate therapeutic repair upon detection of a specific indicator RNA (such as type of cell).

Measuring RNA levels are important for understanding biological systems. Transzystors can be an extremely sensitive and lightweight method for characterizing RNAs in real-time in a biological system. The most useful applications of transzystors are likely for studying systems which are genetically difficult to manipulate or where the genetic manipulation will alter the system behavior. Viruses and phages are an example of systems where making genetic changes are extremely likely to affect the behavior. Instead of genetically changing the virus, transzystors in the host cell can measure viral RNA levels without affecting the behavior of the virus.

### Conclusion

Transzystors use splicing ribozymes to couple the reading of trans-RNA to the writing of RNA, enabling an all-RNA logic where the inputs, the control elements, and the outputs are RNA. The design of transzystors is simple, scalable, and modular and holds promise for novel applications.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

All definitions, as defined and used herein, should be understood to control over to dictionary definitions, definitions in documents incorporated by reference, and/or ordinary meanings of the defined terms.

The indefinite articles “a” and “an”, as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B”, when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently, “at least one of to A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one act, the order of the acts of the method is not necessarily limited to the order in which the acts of the method are recited.

### REFERENCES

[1] P. L. Adams, M. R. Stahley, A. B. Kosek, J. Wang, and S. A. Strobel. Crystal structure of a self-splicing group I intron with both exons. Nature, 430(6995): 45-50, July 2004. doi: 10.1038/nature02642. 2.1.1
[2] R. C. Alexander, D. A. Baum, and S. M. Testa. 5′ transcript replacement in vitro catalyzed by a group I intron-derived ribozyme. Biochemistry, 44(21): 7796-7804, May 2005. doi: 10.1021/biO47284a. 5.5.1, 9.2.3
[3] S. Altuvia and E. G. Wagner. Switching on and off with RNA. Proceedings of the National Academy of Sciences of the United States of America, 97(18): 9824-9826, August 2000. doi: 10.1073/pnas.97.18.9824. 9.2.1
[4] J. B. Andersen, C. Sternberg, L. K. Poulsen, S. P. Bjorn, M. Givskov, and S. Molin. New unstable variants of green fluorescent protein for studies of transient gene expression in bacteria. Appl. Environ. Microbiol., 64(6): 2240-2246, June 1998. 3.2.2, 3.3.3, 4.2.2
[5] A. M. Anderson and J. P. Staley. Long-distance splicing. Proceedings of the National Academy of Sciences of the United States of America, 105(19): 6793-6794, May 2008. doi: 10.1073/pnas.0803068105. 1.1.1
[6] S. Atsumi, Y. Ikawa, H. Shiraishi, and T. Inoue. Design and development of a catalytic ribonucleoprotein. The EMBO journal, 20(19):5453-5460, October 2001. 9.1.4
[7] B. G. Ayre, U. Kohler, H. M. Goodman, and J. Haseloff. Design of highly specific cytotoxins by using trans-splicing ribozymes. Proceedings of the National Academy of Sciences of the United States of America, 96(7): 3507-3512, March 1999. 2.3
[8] B. G. Ayre, U. Kohler, R. Turgeon, and J. Haseloff. Optimization of trans-splicing ribozyme efficiency and specificity by in vivo genetic selection. Nucleic acids research, 30(24), December 2002. 2.3, 5.5.4, 6.4.1
[9] J. R. Babendure, S. R. Adams, and R. Y. Tsien. Aptamers switch on fluorescence of triphenylmethane dyes. Journal of the American Chemical Society, 125(48):14716-14717, December 2003. doi: 10.1021/ja037994o. 9.2.5
[10] D. P. Bartel and J. W. Szostak. Isolation of new ribozymes from a large pool of random sequences. Science (New York, N.Y.), 261(5127):1411-1418, September 1993. 9.2.3
[11] D. J. Battle and J. A. Doudna. Specificity of RNA-RNA helix recognition. Proceedings of the National Academy of Sciences of the United States of America, 99(18):11676-11681, September 2002. doi: 10.1073/pnas.182221799. 5.3
[12] D. A. Baum and S. M. Testa. In vivo excision of a single targeted nucleotide from an mRNA by a trans excision-splicing ribozyme. RNA (New York, N.Y.), 11(6):897-905, June 2005. doi: 10.1261/rna.2050505. 9.2.3
[13] D. A. Baum, J. Sinha, and S. M. Testa. Molecular recognition in a trans excision-splicing ribozyme: non-Watson-Crick base pairs at the 5′ splice site and omegaG at the 3′ splice site can play a role in determining the binding register of reaction substrates. Biochemistry, 44(3):1067-1077, January 2005. doi: 10.1021/biO482304. 5.5.1, 9.2.3
[14] A. A. Beaudry and G. F. Joyce Minimum secondary structure requirements for catalytic activity of a self-splicing group I intron. Biochemistry, 29(27): 6534-6539, July 1990. 5.5.4
[15] M. D. Been and A. T. Perrotta. Group I intron self-splicing with adenosine: evidence for a single nucleoside-binding site. Science (New York, N.Y.), 252 (5004):434-437, April 1991. 5.3
[16] M. Belfort, V. Derbyshire, M. M. Parker, B. Cousineau, and A. M. Lambowitz. Mobile introns: pathways and proteins. In N. L. Craig, R. Craigie, M. Gellert, and A. M. Lambowitz, editors, Mobile DNA II, chapter 31. ASM Press, 2002. 9.2.4
[17] M. A. Bell, A. K. Johnson, and S. M. Testa. Ribozyme-catalyzed excision of targeted sequences from within RNAs. Biochemistry, 41(51):15327-15333, December 2002. 9.2.3
[18] M. A. Bell, J. Sinha, A. K. Johnson, and S. M. Testa. Enhancing the second step of the trans excision-splicing reaction of a group I ribozyme by exploiting P9.0 and P10 for intermolecular recognition. Biochemistry, 43(14):4323-4331, April 2004. doi: 10.1021/biO35874n. 9.2.3
[19] N. H. Bergman, N. C. Lau, V. Lehnert, E. Westhof, and D. P. Bartel. The three-dimensional architecture of the class I ligase ribozyme. RNA (New York, N.Y.), 10(2):176-184, February 2004. 9.2.3
[20] R. R. Breaker. Natural and engineered nucleic acids as tools to explore biology. Nature, 432(7019):838-845, December 2004. doi: 10.1038/nature03195. 1.1.2, 9.1.3
[21] R. R. Breaker. Complex Riboswitches. Science, 319(5871):1795-1797, March 2008. doi: 10.1126/science.1152621. 1.1.2, 9.1.3
[22] J. M. Burke, K. D. Irvine, K. J. Kaneko, B. J. Kerker, B. A. Oettgen, W. M. Tierney, C. L. Williamson, A. J. Zaug, and T. R. Cech. Role of conserved sequence elements 9L and 2 in self-splicing of the Tetrahymena ribosomal RNA precursor. Cell, 45(2):167-176, April 1986. doi: 10.1016/0092-8674(86)90380-6. 5.3
[23] A. R. Buskirk, P. D. Kehayova, A. Landrigan, and D. R. Liu. In vivo evolution of an RNA-based transcriptional activator. Chem Biol, 10(6): 533-540, June 2003. 9.1.3
[24] A. R. Buskirk, A. Landrigan, and D. R. Liu. Engineering a ligand-dependent RNA transcriptional activator. Chem Biol, 11(8):1157-1163, August 2004. doi: 10.1016/j.chembio1.2004.05.017. 9.1.3
[25] J. Byun, N. Lan, M. Long, and B. A. Sullenger. Efficient and specific repair of sickle beta-globin RNA by trans-splicing ribozymes. RNA (New York, N.Y.), 9(10):1254-1263, October 2003. 2.3, 6.4.2, 9.2.1
[26] S. Cabantous, T. C. Terwilliger, and G. S. Waldo. Protein tagging and detection with engineered self-assembling fragments of green fluorescent protein. Nature Biotechnology, 23(1):102-107, December 2004. doi: 10.1038/nbt1044. 4.2.1, 7.2.1, 9.1.7
[27] T. B. Campbell and T. R. Cech. Identification of ribozymes within a ribozyme library that efficiently cleave a long substrate RNA. RNA, 1(6):598-609, August 1995. 4.1, 7.2.3
[28] B. Canton. Engineering the interface between cellular chassis and synthetic biological systems. PhD thesis, Massachusetts Institute of Technology, May 2008. 9.1.3
[29] B. Canton, A. Labno, and D. Endy. Refinement and standardization of synthetic biological parts and devices. Nat Biotech, 26(7):787-793, July 2008. doi: 10.1038/nbt1413. 3.2.1, 8.2
[30] M. G. Caprara, V. Lehnert, A. M. Lambowitz, and E. Westhof. A tyrosyl-tRNA synthetase recognizes a conserved tRNA-like structural motif in the group I intron catalytic core. Cell, 87(6):1135-1145, December 1996. 9.2.6
[31] J. H. Cate, A. R. Gooding, E. Podell, K. Zhou, B. L. Golden, C. E. Kundrot, T. R. Cech, and J. A. Doudna. Crystal structure of a group I ribozyme domain: principles of RNA packing. Science, 273(5282):1678-1685, September 1996. doi: 10.1126/science.273.5282.1678. 2.1.1, 5.3
[32] T. R. Cech. Self-splicing of group I introns. Annual Review of Biochemistry, 59(1):543-568, 1990. doi: 10.1146/annurev.bi.59.070190.002551. 2.1, 2.1.1, 2.2.1, 2.2.1, 2.2.2, 6.4.1
[33] T. R. Cech and B. L. Golden. Building a catalytic active site using only RNA. In R. F. Gesteland, T. R. Cech, and J. F. Atkins, editors, The RNA World, chapter 13, pages 321-349. Cold Spring Harbor Laboratory Press, 2nd edition, 1999. 2.2.3
[34] T. R. Cech, A. J. Zaug, and P. J. Grabowski. In vitro splicing of the ribosomal RNA precursor of Tetrahymena: involvement of a guanosine nucleotide in the excision of the intervening sequence. Cell, 27(3 Pt 2): 487-496, December 1981. 2.2.1
[35] M. Costa and F. Michel. Frequent use of the same tertiary motif by self-folding RNAs. EMBO J, 14(6):1276-1285, March 1995. 5.3
[36] S. Couture, A. D. Ellington, A. S. Gerber, J. M. Chemy, J. A. Doudna, R. Green, M. Hanna, U. Pace, J. Rajagopal, and J. W. Szostak. Mutational analysis of conserved nucleotides in a self-splicing group I intron. J Mol Biol, 215(3):345-358, October 1990. 5.3
[37] G. Di Segni, S. Gastaldi, and G. P. P. Tocchini-Valentini. Cis- and trans-splicing of mRNAs mediated by tRNA sequences in eukaryotic cells. Proceedings of the National Academy of Sciences of the United States of America, May 2008. doi: 10.1073/pnas.0800420105. 9.2.2
[38] C. M. Diges and O. C. Uhlenbeck. Escherichia coli DbpA is an RNA helicase that requires hairpin 92 of 23S rRNA. EMBO J, 20(19):5503-5512, October 2001. 9.2.6
[39] E. A. Doherty and J. A. Doudna. The P4-P6 domain directs higher order folding of the Tetrahymena ribozyme core. Biochemistry, 36(11):3159-3169, March 1997. doi: 10.1021/bi962428+. 9.1.4
[40] J. A. Doudna and T. R. Cech. Self-assembly of a group I intron active site from its component tertiary structural domains. RNA (New York, N.Y.), 1 (1):36-45, March 1995. 9.1.4
[41] J. A. Doudna, B. P. Cormack, and J. W. Szostak. RNA Structure, Not Sequence, Determines the 5′ Splice-Site Specificity of a Group I Intron. Proceedings of the National Academy of Sciences, 86(19):7402-7406, October 1989. doi: 10.1073/pnas.86.19.7402. 4.4.3
[42] W. D. Downs and T. R. Cech. A tertiary interaction in the Tetrahymena intron contributes to selection of the 5′ splice site. Genes Dev., 8(10): 1198-1211, May 1994. doi: 10.1101/gad.8.10.1198. 5.3
[43] T. Durfee, R. Nelson, S. Baldwin, G. Plunkett, V. Burland, B. Mau, J. F. Petrosino, X. Qin, D. M. Muzny, M. Ayele, R. A. Gibbs, B. Csorgo, G. Posfai, G. M. Weinstock, and F. R. Blattner. The complete genome sequence of Escherichia coli DH10B: insights into the biology of a laboratory workhorse. J. Bacteriol., 190(7):2597-2606, April 2008. doi: 10.1128/JB.01695-07. 3.3.1
[44] C. Einvik, M. Elde, and S. Johansen. Group I twintrons: genetic elements in myxomycete and schizopyrenid amoebo agellate ribosomal DNAs. Journal of Biotechnology, 64(1):63-74, September 1998. 9.1.2
[45] C. Einvik, H. Nielsen, E. Westhof, F. Michel, and S. Johansen. Group I-like ribozymes with a novel core organization perform obligate sequential hydrolytic cleavages at two processing sites. RNA (New York, N.Y.), 4(5): 530-541, May 1998. 2.2.1
[46] E. H. Ekland, J. W. Szostak, and D. P. Bartel. Structurally complex and highly active RNA ligases derived from random RNA sequences. Science (New York, N.Y.), 269(5222):364-370, July 1995. 9.2.3
[47] M. B. Elowitz and S. Leibler. A synthetic oscillatory network of transcriptional regulators. Nature, 403(6767):335-338, January 2000. doi: 10.1038/35002125. 1.1.1
[48] D. Endy. Foundations for engineering biology. Nature, 438(7067):449-453, November 2005. doi: 10.1038/nature04342. 1.1.1
[49] T. Fiskaa, E. W. Lundblad, J. R. Henriksen, S. D. Johansen, and C. Einvik. RNA reprogramming of alpha-mannosidase mRNA sequences in vitro by myxomycete group IC1 and IE ribozymes. FEBS Journal, 273(12):2789-2800, June 2006. doi: 10.1111/j.1742-4658.2006.05295.x. 4.1, 5.5.1
[50] E. Ford and M. Ares. Synthesis of circular RNA in bacteria and yeast using RNA cyclase ribozymes derived from a group I intron of phage T4. Proceedings of the National Academy of Sciences of the United States of America, 91(8):3117-3121, April 1994. 9.1.11
[51] T. Franch, M. Petersen, E. G. Wagner, J. P. Jacobsen, and K. Gerdes. Antisense RNA regulation in prokaryotes: rapid RNA/RNA interaction facilitated by a general U-turn loop structure. J Mol Biol, 294(5):1115-1125, December 1999. doi: 10.1006/jmbi.1999.3306. 9.2.1
[52] A. Gampel, M. Nishikimi, and A. Tzagoloff. CBP2 protein promotes in vitro excision of a yeast mitochondrial group I intron. Molecular and cellular biology, 9(12):5424-5433, December 1989. 5.5.1, 9.1.4
[53] T. S. Gardner, C. R. Cantor, and J. J. Collins Construction of a genetic toggle switch in Escherichia coli. Nature, 403(6767):339-342, January 2000. doi: 10.1038/35002131. 1.1.1
[54] B. L. Golden, A. R. Gooding, E. R. Podell, and T. R. Cech. A preorganized active site in the crystal structure of the Tetrahymena ribozyme. Science, 282 (5387):259-264, October 1998. doi: 10.1126/science.282.5387.259. 2.1.1
[55] J. Gorodkin, L. J. Heyer, S. Brunak, and G. D. Stormo. Displaying the information contents of structural RNA alignments: the structure logos. Comput Appl Biosci, 13(6):583-586, December 1997. 5.2, 5.2
[56] D. Grate and C. Wilson. Laser-mediated, site-specific inactivation of RNA transcripts. Proceedings of the National Academy of Sciences of the United States of America, 96(11):6131-6136, May 1999. 9.2.5
[57] S. Griffiths-Jones, S. Moxon, M. Marshall, A. Khanna, S. R. Eddy, and A. Bateman. Rfam: annotating non-coding RNAs in complete genomes. Nucleic Acids Res, 33 (Database issue), January 2005. 2.1
[58] R. Grossberger, O. Mayer, C. Waldsich, K. Semrad, S. Urschitz, and R. Schroeder. Influence of RNA structural stability on the RNA chaperone activity of the Escherichia coli protein StpA. Nucleic acids research, 33(7): 2280-2289, 2005. 9.2.6
[59] A. R. R. Gruber, R. Lorenz, S. H. H. Bernhart, R. Neubock, and I. L. L. Hofacker. The Vienna RNA Websuite. Nucleic acids research, April 2008. 4.2.2
[60] M. Gruen, K. Chang, I. Serbanescu, and D. R. Liu. An in vivo selection system for homing endonuclease activity. Nucleic acids research, 30(7), April 2002. 9.2.4
[61] F. Guo and T. R. Cech. In vivo selection of better self-splicing introns in Escherichia coli: the role of the P1 extension helix of the Tetrahymena intron. RNA, 8(5):647-658, May 2002. 4.1, 4.4.1
[62] F. Guo, A. R. Gooding, and T. R. Cech. Structure of the Tetrahymena ribozyme: base triple sandwich and metal ion at the active site. Molecular Cell, 16(3):351-362, November 2004. doi: 10.1016/j.molce1.2004.10.003. 2.1.1, 5.3
[63]H. Guo, M. Karberg, M. Long, Jones, B. Sullenger, and A. M. Lambowitz. Group II introns designed to insert into therapeutically relevant DNA target sites in human cells. Science, 289(5478):452-457, July 2000. doi: 10.1126/science.289.5478.452. 9.2.4
[64] S. Hasegawa and J. Rao. Modulating the splicing activity of Tetrahymena ribozyme via RNA self-assembly. FEBS Letters, 580(6):1592-1596, March 2006. doi: 10.1016/j.febslet.2006.01.090. 5.3
[65] S. Hasegawa, G. Gowrishankar, and J. Rao. Detection of mRNA in mammalian cells with a split ribozyme reporter. Chembiochem: a European journal of chemical biology, 7(6):925-928, June 2006. doi: 10.1002/cbic.200600061. 8.1.1, 8.4.1
[66] J. Hasty, D. McMillen, and J. J. Collins. Engineered gene circuits. Nature, 420(6912):224-230, November 2002. doi: 10.1038/nature01257. 1.1.1
[67] P. Haugen, M. Andreassen, A. B. Birgisdottir, and S. Johansen. Hydrolytic cleavage by a group I intron ribozyme is dependent on RNA structures not important for splicing. European journal of biochemistry/FEBS, 271(5): 1015-1024, March 2004. 6.4.2
[68] E. J. Hayden, C. A. Riley, A. S. Burton, and N. Lehman. RNA-directed construction of structurally complex and active ligase ribozymes through recombination. RNA, 11(11):1678-1687, November 2005. doi: 10.1261/rna.2125305. 5.5.1, 9.1.2, 9.2.3
[69] D. Herschlag. Implications of ribozyme kinetics for targeting the cleavage of specific RNA molecules in vivo: more isn't always better. Proceedings of the National Academy of Sciences of the United States of America, 88(16): 6921-6925, August 1991. 9.2.1
[70] D. Herschlag. Evidence for processivity and two-step binding of the RNA substrate from studies of J1/2 mutants of the Tetrahymena ribozyme. Biochemistry, 31(5):1386-1399, February 1992. 5.3
[71] M. Hirabayashi, S. Taira, S. Kobayashi, K. Konishi, K. Katoh, Y. Hiratsuka, M. Kodaka, T. Q. Uyeda, N. Yumoto, and T. Kubo. Malachite green-conjugated microtubules as mobile bioprobes selective for malachite green aptamers with capturing/releasing ability. Biotechnology and bioengineering, 94(3):473-480, June 2006. doi: 10.1002/bit.20867. 9.2.5
[72] J. L. Hougland, R. N. Sengupta, Q. Dai, S. K. Deb, and J. A. Piccirilli. The 2′-hydroxyl group of the guanosine nucleophile donates a functionally important hydrogen bond in the Tetrahymena ribozyme reaction. Biochemistry, June 2008. doi: 10.1021/bi8000648. 2.2.1
[73] M. A. J. A. Iafolla, M. Mazumder, V. Sardana, T. Velauthapillai, K. Pannu, and D. R. R. Mcmillen. Dark proteins: Effect of inclusion body formation on quantification of protein expression. Proteins, March 2008. doi: 10.1002/prot.22024. 4.4.3
[74] Y. Ikawa, H. Ohta, H. Shiraishi, and T. Inoue. Long-range interaction between the P2.1 and P9.1 peripheral domains of the Tetrahymena ribozyme. Nucleic Acids Res, 25(9):1761-1765, May 1997. 5.3
[75] Y. Ikawa, W. Yoshioka, Y. Ohki, H. Shiraishi, and T. Inoue. Self-splicing of the Tetrahymena group I ribozyme without conserved base-triples. Genes to Cells, 6(5):411-420, May 2001. 9.1.4
[76] T. Inoue and Y. Ikawa. Activation of the group I intron ribozymes with their peripheral domains. In G. Krupp and R. K. Gaur, editors, Ribozyme: Biochemistry and Biotechnology, chapter 2, pages 27-39. Eaton Publishing, 2000. 9.1.4
[77] F. J. Isaacs, D. J. Dwyer, C. Ding, D. D. Pervouchine, C. R. Cantor, and J. J. Collins. Engineered riboregulators enable post-transcriptional control of gene expression. Nat Biotechnol, 22(7):841-847, July 2004. doi: 10.1038/nbt986. 1.1.1, 1.1.2, 8.1.1, 9.1.1
[78] F. J. Isaacs, D. J. Dwyer, and J. J. Collins. RNA synthetic biology. Nature Biotechnology, 24(5):545-554, May 2006. doi: 10.1038/nbt1208. 1.1.2, 9.1.3
[79] S. A. Jackson, S. Koduvayur, and S. A. Woodson. Self-splicing of a group I intron reveals partitioning of native and misfolded RNA populations in yeast. RNA (New York, N.Y.), 12(12):2149-2159, December 2006. doi: 10.1261/rna.184206. 4.2.2, 4.4.3
[80] S. Johansen and P. Haugen. A new nomenclature of group I introns in ribosomal DNA. RNA (New York, N.Y.), 7(7):935-936, July 2001. 2.1
[81] A. K. Johnson, J. Sinha, and S. M. Testa. Trans insertion-splicing: ribozyme-catalyzed insertion of targeted sequences into RNAs. Biochemistry, 44(31):10702-10710, August 2005. doi: 10.1021/bi0504815. 9.2.3
[82] J. M. Johnson, J. Castle, P. Garrett-Engele, Z. Kan, P. M. Loerch, C. D. Armour, R. Santos, E. E. Schadt, R. Stoughton, and D. D. Shoemaker. Genome-wide survey of human alternative pre-mRNA splicing with exon junction microarrays. Science, 302(5653):2141-2144, December 2003. doi: 10.1126/science.1090100. 1.1.1
[83] T. H. Johnson, P. Tijerina, A. B. Chadee, D. Herschlag, and R. Russell. Structural specificity conferred by a group I RNA peripheral element. Proceedings of the National Academy of Sciences of the United States of America, 102(29):10176-10181, July 2005. doi: 10.1073/pnas.0501498102. 5.4.1
[84] W. K. Johnston, P. J. Unrau, M. S. Lawrence, M. E. Glasner, and D. P. Bartel. RNA-catalyzed RNA polymerization: accurate and general RNA-templated primer extension. Science (New York, N.Y.), 292(5520): 1319-1325, May 2001. doi: 10.1126/science.1060786. 9.2.3
[85] J. P. Jones, M. N. Kierlin, R. G. Coon, J. Perutka, A. M. Lambowitz, and B. A. Sullenger. Retargeting mobile group II introns to repair mutant genes. Mol Ther, 11(5):687-694, May 2005. doi: 10.1016/j.ymthe.2005.01.014. 9.2.4
[86] M. Karberg, H. Guo, J. Zhong, R. Coon, J. Perutka, and A. M. Lambowitz. Group II introns as controllable gene targeting vectors for genetic manipulation of bacteria. Nat Biotech, 19(12):1162-1167, December 2001. doi: 10.1038/nbt1201-1162. 9.2.4
[87] D.-S. Kim, V. Gusti, S. G. Pillai, and R. K. Gaur. An artificial riboswitch for controlling pre-mRNA splicing. RNA, 11(11):1667-1677, November 2005. doi: 10.1261/rna.2162205. 9.1.9
[88] S. P. Koduvayur and S. A. Woodson. Intracellular folding of the Tetrahymena group I intron depends on exon sequence and promoter choice. RNA (New York, N.Y.), 10(10):1526-1532, October 2004. doi: 10.1261/rna.7880404. 2.2.3, 4.4.3
[89] U. Kohler, B. G. Ayre, H. M. Goodman, and J. Haseloff. Trans-splicing ribozymes for targeted gene delivery. Journal of Molecular Biology, 285(5): 1935-1950, February 1999. doi: 10.1006/jmbi.1998.2447. 2.2.1, 2.3, 4.1, 5.5.4, 6.1.1, 6.2.1, 6.3.2, 9.2.1
[90] D. M. Kolpashchikov. Binary malachite green aptamer for uorescent detection of nucleic acids. Journal of the American Chemical Society, 127(36): 12442-12443, September 2005. doi: 10.1021/ja0529788. 9.2.5
[91] A. M. Lambowitz and M. G. Caprara. Group I and group II ribozymes as RNPs: clues to the past and guides to the future. In R. F. Gesteland, T. R. Cech, and J. F. Atkins, editors, The RNA World, chapter 18, pages 451-485. Cold Spring Harbor Laboratory Press, 2nd edition, 1999. 5.4.1, 9.1.4, 9.2.2
[92] N. Lan, R. P. Howrey, S. W. Lee, C. A. Smith, and B. A. Sullenger. Ribozyme-mediated repair of sickle beta-globin mRNAs in erythrocyte precursors. Science, 280(5369):1593-1596, June 1998. doi: 10.1126/science.280.5369.1593. 2.3
[93] N. Lan, B. L. Rooney, S. W. Lee, R. P. Howrey, C. A. Smith, and B. A. Sullenger. Enhancing RNA repair efficiency by combining trans-splicing ribozymes that recognize different accessible sites on a target RNA. Molecular therapy, 2(3):245-255, September 2000. doi: 10.1006/mthe.2000.0125. 6.4.1, 6.4.2
[94] R. A. Lease, M. E. Cusick, and M. Belfort. Riboregulation in Escherichia coli: DsrA RNA acts by RNA:RNA interactions at multiple loci. Proceedings of the National Academy of Sciences of the United States of America, 95(21): 12456-12461, October 1998. 1.1.2
[95] J. H. Lee and A. Pardi. Thermodynamics and kinetics for base-pair opening in the P1 duplex of the Tetrahymena group I ribozyme. Nucleic acids research, 35(9):2965-2974, 2007. 2.2.1, 4.4.3
[96] P. Legault, D. Herschlag, D. W. Celander, and T. R. Cech. Mutations at the guanosine-binding site of the Tetrahymena ribozyme also affect site-specific hydrolysis. Nucleic acids research, 20(24):6613-6619, December 1992. 2.2.1, 2.2.2, 3.1.2, 6.4.1, 6.4.1
[97] V. Lehnert, L. Jaeger, F. Michele, and E. Westhof. New loop-loop tertiary interactions in self-splicing introns of subgroup IC and ID: a complete 3D model of the Tetrahymena thermophila ribozyme. Chemistry & Biology, 3 (12):993-1009, December 1996. 4.4.3, 5.3
[98] M. B. Long, J. P. Jones, B. A. Sullenger, and J. Byun. Ribozyme-mediated revision of RNA and DNA. Journal of clinical investigation, 112(3):312-318, August 2003. doi: 10.1172/JCI19386. 2.3, 9.2.4
[99] E. W. Lundblad, P. Haugen, and S. D. Johansen. Trans-splicing of a mutated glycosylasparaginase mRNA sequence by a group I ribozyme deficient in hydrolysis. European Journal of Biochemistry, 271(23-24):4932+, 2004. doi: 10.1111/j.1432-1033.2004.04462.x. 5.5.1, 6.4.2
[100] M. Mandal, M. Lee, J. E. Barrick, Z. Weinberg, G. M. Emilsson, W. L. Ruzzo, and R. R. Breaker. A Glycine-Dependent Riboswitch That Uses Cooperative Binding to Control Gene Expression. Science, 306(5694):275-279, October 2004. doi: 10.1126/science. 1100829. 8.4.3
[101] S. G. Mansfield, R. H. Clark, M. Puttaraju, J. Kole, J. A. Cohn, L. G. Mitchell, and M. A. Garcia-Blanco. 5′ exon replacement and repair by spliceosome-mediated RNA trans-splicing. RNA (New York, N.Y.), 9(10): 1290-1297, October 2003. 9.2.2
[102] O. Mayer, L. Rajkowitsch, C. Lorenz, R. Konrat, and R. Schroeder. RNA chaperone activity and RNA-binding properties of the E. coli protein StpA. Nucleic acids research, 35(4):1257-1269, 2007. 9.2.6
[103] K. E. McGinness and G. F. Joyce. RNA-catalyzed RNA ligation on an external RNA template. Chemistry & biology, 9(3):297-307, March 2002. 9.2.3
[104] F. Michel, M. Hanna, R. Green, D. P. Bartel, and J. W. Szostak. The guanosine binding site of the Tetrahymena ribozyme. Nature, 342(6248): 391-395, November 1989. doi: 10.1038/342391a0.2.2.1, 3.1.2, 9.1.2
[105] F. Michel, A. D. Ellington, S. Couture, and J. W. Szostak. Phylogenetic and genetic evidence for base-triples in the catalytic domain of group I introns. Nature, 347(6293):578-580, October 1990. doi: 10.1038/347578a0.5.3
[106] G. Mohr, M. G. Caprara, Q. Guo, and A. M. Lambowitz. A tyrosyl-tRNA synthetase can function similarly to an RNA structure in the Tetrahymena ribozyme. Nature, 370(6485):147-150, July 1994. doi: 10.1038/370147a0.5.4.1
[107] G. Mohr, D. Smith, M. Belfort, and A. M. Lambowitz. Rules for DNA target-site recognition by a lactococcal group II intron enable retargeting of the intron to specific DNA sequences. Genes & development, 14(5):559-573, March 2000. 9.2.4
[108] F. L. Murphy and T. R. Cech. Alteration of substrate specificity for the endoribonucleolytic cleavage of RNA by the Tetrahymena ribozyme. Proc Natl Acad Sci USA, 86(23):9218-9222, December 1989. 4.4.3
[109] F. L. Murphy and T. R. Cech. GAAA tetraloop and conserved bulge stabilize tertiary structure of a group I intron domain. Journal of molecular biology, 236(1):49-63, February 1994. doi: 10.1006/jmbi.1994.1117. 5.3
[110] F. C. Neidhardt, P. L. Bloch, and D. F. Smith. Culture medium for enterobacteria. J Bacteriol, 119(3):736-747, September 1974. 3.3.1
[111] S. Oberdoerfler, L. F. Moita, D. Neems, R. P. Freitas, N. Hacohen, and A. Rao. Regulation of CD45 alternative splicing by heterogeneous ribonucleoprotein, hnRNPLL. Science, 321(5889):686-691, August 2008. doi: 10.1126/science.1157610. 1.1.1
[112] Y. Oe, Y. Ikawa, H. Shiraishi, and T. Inoue. Analysis of the P7 region within the catalytic core of the Tetrahymena ribozyme by employing in vitro selection. Nucleic Acids Symp Ser, 44(1):197-198, 2000. 5.5.4
[113] Y. Oe, Y. Ikawa, H. Shiraishi, and T. Inoue. Conserved base-pairings between C266-A268 and U307-G309 in the P7 of the Tetrahymena ribozyme is nonessential for the in vitro self-splicing reaction. Biochem Biophys Res Commun, 284(4):948-954, June 2001. doi: 10.1006/bbrc.2001.5072. 5.5.4, 5.3
[114] J. Pan and S. A. Woodson. Folding intermediates of a self-splicing RNA: mispairing of the catalytic core. Journal of molecular biology, 280(4):597-609, July 1998. doi: 10.1006/jmbi.1998.1901. 4.2.2, 4.4.3, 5.5.4, 5.3
[115] T. Pan and T. Sosnick. RNA folding during transcription. Annual review of biophysics and biomolecular structure, 35:161-175, 2006. doi: 10.1146/annurev.biophys.35.040405.102053. 2.2.3, 4.4.3
[116] M. Parisien and F. Major. The MC-Fold and MC-Sym pipeline infers RNA structure from sequence data. Nature, 452(7183):51-55, 2008. doi: 10.1038/nature06684. 8.4.1
[117] R. Penchovsky and R. R. Breaker. Computational design and experimental validation of oligonucleotide-sensing allosteric ribozymes. Nature Biotechnology, 23(11):1424-1433, October 2005. doi: 10.1038/nbt1155. 8.4.1
[118] R. Perriman and M. Ares. Circular mRNA can direct translation of extremely long repeating-sequence proteins in vivo. RNA (New York, N.Y.), 4(9): 1047-1054, September 1998. 9.1.11
[119] A. Peyman. P2 functions as a spacer in the Tetrahymena ribozyme. Nucleic Acids Res, 22(8):1383-1388, April 1994. 4.4.3, 5.3
[120] J. V. Price and T. R. Cech. Coupling of Tetrahymena ribosomal RNA splicing to beta-galactosidase expression in Escherichia coli. Science (New York, N.Y.), 228(4700):719-722, May 1985. 5.3
[121] M. Puttaraju and M. D. Been. Circular ribozymes generated in Escherichia coli using group I self-splicing permuted intron-exon sequences. Journal of biological chemistry, 271(42):26081-26087, October 1996. 9.1.11
[122] A. M. Pyle, S. Moran, S. A. Strobel, T. Chapman, D. H. Turner, and T. R. Cech. Replacement of the conserved G.U with a G-C pair at the cleavage site of the Tetrahymena ribozyme decreases binding, reactivity, and fidelity. Biochemistry, 33(46):13856-13863, November 1994. 2.2.1
[123] L. Rajkowitsch, D. Chen, S. Stamp, K. Semrad, C. Waldsich, O. Mayer, M. F. Jantsch, R. Konrat, U. Blasi, and R. Schroeder. RNA chaperones, RNA annealers and RNA helicases. RNA biology, 4(3):118-130, November 2007. 9.2.1, 9.2.6
[124] M. A. Reynolds, K. Kastury, J. Groskopf, J. A. Schalken, and H. Rittenhouse. Molecular markers for prostate cancer. Cancer letters, 249(1):5-13, April 2007. doi: 10.1016/j.canlet.2006.12.029. 8.4.4
[125] C. A. Riley and N. Lehman. Generalized RNA-directed recombination of RNA.
Chemistry & biology, 10(12):1233-1243, December 2003. 5.5.1, 9.1.2, 9.2.3
[126] C. S. Rogers, C. G. Vanoye, B. A. Sullenger, and A. L. George. Functional repair of a mutant chloride channel using a trans-splicing ribozyme. Journal of clinical investigation, 110(12):1783-1789, December 2002. doi: 10.1172/JCI16481. 2.3, 6.4.2
[127] K. J. Ryu, J. H. Kim, and S. W. Lee. Ribozyme-mediated selective induction of new gene activity in hepatitis C virus internal ribosome entry site-expressing cells by targeted trans-splicing. Molecular therapy, 7(3): 386-395, March 2003. 2.3, 9.1.6
[128] L. Sandegren and B. M. Sjoberg. Self-splicing of the bacteriophage T4 group I introns requires efficient translation of the pre-mRNA in vivo and correlates with the growth state of the infected bacterium. Journal of bacteriology, 189 (3):980-990, February 2007. doi: 10.1128/JB.01287-06. 4.4.3, 6.4.2
[129] T. D. Schneider and R. M. Stephens. Sequence logos: a new way to display consensus sequences. Nucleic Acids Res, 18(20):6097-6100, October 1990. 5.2
[130] E. A. Schultes and D. P. Bartel. One sequence, two ribozymes: implications for the emergence of new ribozyme folds. Science, 289(5478):448-452, July 2000. doi: 10.1126/science.289.5478.448. 5.1
[131] K. Semrad and R. Schroeder. A ribosomal function is necessary for efficient splicing of the T4 phage thymidylate synthase intron in vivo. Genes & development, 12(9):1327-1337, May 1998. 4.4.3, 6.4.2
[132] A. Serganov and D. J. Patel. Ribozymes, riboswitches and beyond: regulation of gene expression without proteins. Nature Reviews Genetics, 8(10):776-790, September 2007. doi: 10.1038/nrg2172. 1.1.2
[133] S. Shan, A. Yoshida, S. Sun, J. A. Piccirilli, and D. Herschlag. Three metal ions at the active site of the Tetrahymena group I ribozyme. Proceedings of the National Academy of Sciences of the United States of America, 96(22): 12299-12304, October 1999. 2.2.1
[134] S. Shan, A. V. Kravchuk, J. A. Piccirilli, and D. Herschlag. Defining the catalytic metal ion interactions in the Tetrahymena ribozyme reaction. Biochemistry, 40(17):5161-5171, May 2001. 2.2.1
[135] N.C. Shaner, R. E. Campbell, P. A. Steinbach, B. N. G. Giepmans, A. E. Palmer, and R. Y. Tsien Improved monomeric red, orange and yellow uorescent proteins derived from Discosoma sp. red uorescent protein. Nature Biotechnology, 22(12):1567+, November 2004. doi: 10.1038/nbt1037. 7.3.1, 7.5.2
[136] Y. Shao, Y. Wu, C. Y. Chan, K. Mcdonough, and Y. Ding. Rational design and rapid screening of antisense oligonucleotides for prokaryotic gene modulation. Nucleic Acids Research, 34(19):5660-5669, November 2006. doi: 10.1093/nadgk1715. 7.2.4
[137] R. P. Shetty. Applying engineering principles to the design and construction of transcriptional devices. PhD thesis, Massachusetts Institute of Technology, May 2008. 1.1.1
[138] R. P. Shetty, D. Endy, and T. F. Knight. Engineering BioBrick vectors from BioBrick parts. Journal of biological engineering, 2(1), 2008. doi: 10.1186/1754-1611-2-5. 3.2.3
[139] K. S. Shin, B. A. Sullenger, and S. W. Lee. Ribozyme-mediated induction of apoptosis in human cancer cells by targeted repair of mutant p53 RNA. Molecular therapy, 10(2):365-372, August 2004. doi: 10.1016/j.ymthe.2004.05.007. 2.3
[140] S. K. Silverman. Rube Goldberg goes (ribo)nuclear? Molecular switches and sensors made from RNA. RNA, 9(4):377-383, April 2003. doi: 10.1261/rna.2200903. 8.1.1, 9.1.9
[141] R. W. Simons and N. Kleckner. Biological regulation by antisense RNA in prokaryotes. Annual review of genetics, 22:567-600, 1988. doi: 10.1146/annurev.ge.22.120188.003031. 6.4.1, 9.2.1
[142] D. Sprinzak and M. B. Elowitz. Reconstruction of genetic circuits. Nature, 438(7067):443-448, 2005. doi: 10.1038/nature04335. 1.1.1
[143] M. R. Stahley and S. A. Strobel. RNA splicing: group I intron crystal structures reveal the basis of splice site selection and metal ion catalysis. Current opinion in structural biology, 16(3):319-326, June 2006. doi: 10.1016/j.sbi.2006.04.005. 2.1.1
[144] M. N. Stojanovic and D. M. Kolpashchikov. Modular aptameric sensors. Journal of the American Chemical Society, 126(30):9266-9270, August 2004. doi: 10.1021/ja032013t. 9.2.5
[145] F. Storici, K. Bebenek, T. A. Kunkel, D. A. Gordenin, and M. A. Resnick. RNA-templated DNA repair. Nature, April 2007. doi: 10.1038/nature05720. 9.2.4
[146] B. A. Sullenger and T. R. Cech. Ribozyme-mediated repair of defective mRNA by targeted trans-splicing. Nature, 371(6498):619-622, October 1994. doi: 10.1038/371619a0. 2.3
[147] A. Tats, M. Remm, and T. Tenson. Highly expressed proteins have an increased frequency of alanine in the second amino acid position. BMC Genomics, 7, 2006. doi: 10.1186/1471-2164-7-28. 7.3.1
[148] N. Toor, K. S. Keating, S. D. Taylor, and A. M. Pyle. Crystal structure of a self-spliced group II intron. Science, 320(5872):77-82, April 2008. doi: 10.1126/science.1153803. 9.2.4
[149] D. K. Treiber, M. S. Rook, P. P. Zarrinkar, and J. R. Williamson. Kinetic intermediates trapped by native interactions in RNA folding. Science, 279 (5358):1943-1946, March 1998. doi: 10.1126/science.279.5358.1943. 5.5.4, 5.3
[150] J. Tsang and G. F. Joyce. Evolutionary optimization of the catalytic properties of a DNA-cleaving ribozyme. Biochemistry, 33(19):5966-5973, May 1994. 9.2.4
[151] J. Tsang and G. F. Joyce. Specialization of the DNA-cleaving activity of a group I ribozyme through in vitro evolution. Journal of molecular biology, 262 (1):31-42, September 1996. doi: 10.1006/jmbi.1996.0496. 9.2.4
[152] R. Y. Tsien. The green uorescent protein. Annu Rev Biochem, 67:509-544, 1998. doi: 10.1146/annurev.biochem. 67.1.509. 4.2.1, 6.2.1
[153] M. Valencia-Burton, R. M. Mccullough, C. R. Cantor, and N. E. Broude. RNA visualization in live bacterial cells using uorescent protein complementation. Nat Meth, 4(5):421-427, May 2007. doi: 10.1038/nmeth1023. 9.1.3, 9.2.5
[154] G. van der Horst and T. Inoue. Requirements of a group I intron for reactions at the 3′ splice site. J Mol Biol, 229(3):685-694, February 1993. doi: 10.1006/jmbi.1993.1072. 2.2.1, 2.2.2, 4.4.2
[155] G. van der Horst, A. Christian, and T. Inoue. Reconstitution of a group I intron self-splicing reaction with an activator RNA. Proceedings of the National Academy of Sciences of the United States of America, 88(1):184-188, January 1991. 5.5.4, 9.1.4
[156] T. Waldminghaus, A. Fippinger, J. Alfsmann, and F. Narberhaus. RNA thermometers are common in alpha- and gamma-proteobacteria. Biol Chem, 386(12):1279-1286, December 2005. doi: 10.1515/BC.2005.145. 1.1.2, 9.1.9
[157] L. Wang, J. Xie, and P. G. Schultz. Expanding the genetic code. Annual Review of Biophysics and Biomolecular Structure, 35(1):225-249, 2006. doi: 10.1146/annurev.biophys.35.101105.121507. 9.1.3
[158] M. Warashina, T. Kuwabara, Y. Kato, M. Sano, and K. Taira. RNA-protein hybrid ribozymes that efficiently cleave any mRNA independently of the structure of the target RNA. Proc Natl Acad Sci USA, 98(10):5572-5577, May 2001. doi: 10.1073/pnas.091411398. 9.2.6
[159] R. B. Waring. Identification of phosphate groups important to self-splicing of the Tetrahymena rRNA intron as determined by phosphorothioate substitution. Nucleic Acids Res, 17(24):10281-10293, December 1989. 5.3
[160] K. P. Williams, D. N. Fujimoto, and T. Inoue. A region of group I introns that contains universally conserved residues but is not essential for self-splicing. Proceedings of the National Academy of Sciences, 89(21): 10400-10404, November 1992. doi: 10.1073/pnas.89.21.10400. 5.3
[161] K. P. Williams, H. Imahori, D. N. Fujimoto, and T. Inoue. Selection of novel forms of a functional domain within the Tetrahymena ribozyme. Nucleic Acids Res, 22(11):2003-2009, June 1994. 5.5.4, 6.4.2, 9.1.4
[162] M. N. Win and C. D. Smolke. A modular and extensible RNA-based gene-regulatory platform for engineering cellular function. Proceedings of the National Academy of Sciences, 104(36):14283-14288, September 2007. doi: 10.1073/pnas.0703961104. 9.1.3, 9.1.9, 9.2.3
[163] S. A. Woodson. Structure and assembly of group I introns. Curr Opin Struct Biol, 15(3):324-330, June 2005. doi: 10.1016/j.sbi.2005.05.007. 2.1.1
[164] M. Wu and I. Tinoco. RNA folding causes secondary structure rearrangement. Proc Natl Acad Sci USA, 95(20):11555-11560, September 1998. 5.4.3, 5.3
[165] A. Xayaphoummine, T. Bucher, and H. Isambert. Kinefold web server for RNA/DNA folding path and structure prediction including pseudoknots and knots. Nucleic Acids Res, 33 (Web Server issue), July 2005. 4.2.2, 8.2.1
[166] A. Xayaphoummine, V. Viasnofi, S. Harlepp, and H. Isambert. Encoding folding paths of RNA switches. Nucleic acids research, 35(2):614-622, 2007. 8.4.2
[167] L. Yen, J. Svendsen, J. S. Lee, J. T. Gray, M. Magnier, T. Baba, R. J. D'Amato, and R. C. Mulligan. Exogenous control of mammalian gene expression through modulation of RNA self-cleavage. Nature, 431(7007): 471-476, September 2004. doi: 10.1038/nature02844. 9.1.9
[168] B. Young, D. Herschlag, and T. R. Cech. Mutations in a nonconserved sequence of the Tetrahymena ribozyme increase activity and specificity. Cell, 67(5):1007-1019, November 1991. doi: 10.1016/0092-8674(91)90373-7. 5.3
[169] P. J. Zamenhof and M. Villarejo. Construction and properties of Escherichia coli strains exhibiting alpha-complementation of beta-galactosidase fragments in vivo. Journal of bacteriology, 110(1):171-178, April 1972. 3.3.5, 7.2.1
[170] P. P. Zarrinkar and J. R. Williamson. Kinetic intermediates in RNA folding. Science (New York, N.Y.), 265(5174):918-924, August 1994. 5.5.4
[171] A. J. Zaug, J. R. Kent, and T. R. Cech. A labile phosphodiester bond at the ligation junction in a circular intervening sequence RNA. Science (New York, N.Y.), 224(4649):574-578, May 1984. 2.2.2
[172] A. Zhang, K. M. Wassarman, J. Ortega, A. C. Steven, and G. Storz. The Sm-like Hfq protein increases OxyS RNA interaction with target mRNAs. Molecular cell, 9(1):11-22, January 2002. 9.2.6
[173] F. Zhang, E. S. Ramsay, and S. A. Woodson. In vivo facilitation of Tetrahymena group I intron splicing in Escherichia coli pre-ribosomal RNA. RNA (New York, N.Y.), 1(3):284-292, May 1995. 2.2.3
[174] S. Zhang, C. Ma, and M. Chalfe. Combinatorial marking of cells and organelles with reconstituted uorescent proteins. Cell, 119(1):137-144, October 2004. doi: 10.1016/j.cell.2004.09.012. 7.2.1, 9.1.7
[175] Y. Zhou, C. Lu, Q. J. Wu, Y. Wang, Z. T. Sun, J. C. Deng, and Y. Zhang. GISSD: Group I Intron Sequence and Structure Database. Nucleic acids research, 36 (Database issue), January 2008. 2.1, 5.2

All publications, patents and sequence database entries mentioned herein, including those items listed below, are hereby incorporated by reference in their entirety as if each individual publication or patent was specifically and individually indicated to be incorporated by reference. In case of conflict, the present application, including any definitions herein, will control.

1. A (conditionally active) ribozyme, comprising
a catalytic RNA fragment that splices one or more RNA molecules, and
at least one regulatory element modulating the activity of said catalytic RNA fragment, wherein said ribozyme catalyzes a cis-splicing reaction and/or a trans-splicing reaction, and
optionally, wherein the nucleotide sequence of the internal guide sequence (IGS) is altered in at least one position.

2.-9. (canceled)

10. The (conditionally active) ribozyme of claim 1,
wherein said at least one regulatory element comprises a nucleotide sequence that reversibly binds to said ribozyme,
optionally, wherein said at least one regulatory element reversibly binds to the internal guide sequence (IGS) of said ribozyme, preferably to the reaction site,
optionally, wherein said binding inhibits the splicing activity of the catalytic RNA fragment of said ribozyme,
optionally, wherein said at least one regulatory element further comprises at least one nucleotide sequence reversibly binding to a target molecule, said binding impairing the binding of said at least one regulatory element to said ribozyme, and
optionally, wherein said target molecule is an amino acid, a peptide, or a protein, a chemical compound, or a nucleic acid molecule.

11.-33. (canceled)

34. A nucleic acid coding for a (conditionally active) ribozyme as claimed in claim 1.

35.-36. (canceled)

37. A cell expressing at least one (conditionally active) ribozyme as claimed in claim 1.

38. A kit comprising
the (conditionally active) ribozyme of claim 1, and/or
a nucleic acid encoding said ribozyme, and/or
a cell expressing said ribozyme.

39.-40. (canceled)

41. A method of splicing of one or more RNA molecules, comprising
contacting one or more RNA molecules with the (conditionally active) ribozyme of claim 1, wherein said (conditionally active) ribozyme splices said one or more RNA molecules.

42.-47. (canceled)

48. A method of changing the state of a cell, comprising
contacting a cell with the (conditionally active) ribozyme of claim 1,
optionally, wherein the (conditionally active) ribozyme binds a target molecule expressed in said cell, and
optionally, wherein said target nucleic acid molecule is an endogenous gene product specifically expressed in said cell,
whereby the (conditionally active) ribozyme changes the state of the cell.

49.-53. (canceled)

54. A method, comprising
contacting a sample with the (conditionally active) ribozyme of claim 1,
wherein said (conditionally active) ribozyme comprises a regulatory element specifically binding a target molecule, said binding modulating the splicing activity of the catalytic RNA fragment of said (conditionally active) ribozyme, said modulating leading to a detectable change in the state of said sample, and wherein the contacting is under conditions that allow said (conditionally active) ribozyme to bind said target molecule.

55.-59. (canceled)

60. The method of claim 54, further comprising
comparing the quantity of change in said sample to the quantity of change in a reference or control sample,
wherein presence or an elevated quantity of change in said sample is indicative of presence or an elevated amount of said target molecule in said sample, and
wherein absence or a decreased quantity of change is indicative of absence or a decreased amount of said target molecule in said sample.

61. The method of claim 54, wherein the sample is a cell or tissue or body fluid sample from a subject, and wherein the presence and/or an increased quantity of change in said sample as compared to a reference or control sample indicates the presence of a condition in said subject, and the absence and/or a decreased quantity of change in said sample as compared to a reference or control sample indicates the absence of a condition in said subject.

62.-74. (canceled)

75. The method of claim 54, wherein two or more (conditionally active) ribozymes are used.

76. The method of claim 75, wherein the splicing activity of at least one of these two or more (conditionally active) ribozymes leads to the generation of a target molecule for at least one of the two or more (conditionally active) ribozymes, resulting in an amplification of the detectable change in the sample, and/or in a change of the quality of the detectable change in the sample.

77.-79. (canceled)

80. A method using a (conditionally active) ribozyme to treat a subject, comprising
administering to said subject the ribozyme of claim 1,
wherein a splicing activity of the (conditionally active) ribozyme modulated specifically by a target molecule indicative of a disease or condition and/or of an undesired cell state causally related to a disease or condition in said subject,
resulting in an amelioration of said disease or condition or of symptoms of said disease or condition.

81.-90. (canceled)

91. The method of claim 80, wherein said disease or condition is an infectious disease, an autoimmune disease, a neoplastic disease, an endocrine autocrine or paracrine disease, a parasitic disease or a genetic disorder.

92. A composition, comprising
one or more (conditionally active) ribozymes as claimed in claim 1, and/or
one or more nucleic acids coding for the one or more (conditionally active) ribozymes, and/or
one or more cells expressing the one or more (conditionally active) ribozymes.

93.-94. (canceled)

95. A method of generating the ribozyme of claim 1, comprising using a computational RNA folding model to predict and/or model the splicing activity of one or more mutations and engineering at least one mutation or alteration in said ribozyme based on the results of said prediction and/or modeling results.

96. (canceled)
